(19) United States
(12) Patent Application Publication
(10) Pub. No.: US 2026/0364098 A1
(43) Pub. Date: Nov. 19, 2026

(54) HETEROARYL COMPOUNDS AS ENDOCANNABINOID SYSTEM MODULATORS AND USES THEREOF

(71) Applicant: Northeastern University, Boston, MA (US)

(72) Inventors: Alexandros Makriyannis, Watertown, MA (US); Kiran Vemuri, Boston, MA (US); Maria Gerasi, Allston, MA (US); Christos Iliopoulos Tsoutsouvas, Allston, MA (US)

(21) Appl. No.: 19/487,288

(22) PCT Filed: Jun. 21, 2024
(86) PCT No.: PCT/US2024/035095

§ 371 Date: Nov. 24, 2025

Related U.S. Application Data
(60) Provisional application No. 63/509,965, filed on Jun. 23, 2023.

(51) Int. Cl. A61K 31/69; A61K 31/404; A61P 25/04; C07D 209/14; C07F 5/02

(52) U.S. Cl. CPC A61K 31/69; A61K 31/404; A61P 25/04; C07D 209/14; C07F 5/025; C07F 5/027

(57) ABSTRACT

Compounds able to modulate the endocannabinoid system, their preparation and method of use in the treatment and prevention of diseases in which modulation of the receptor is beneficial are described. Also described are pharmaceutically acceptable compositions comprising the disclosed compounds and methods of using said compositions in the treatment of various disorders, diseases, or conditions, in which modulation of the endocannabinoid system is beneficial.

### RELATED APPLICATIONS

This application claims the benefit of U.S. Provisional Application No. 63/509,965, filed on Jun. 23, 2023. The entire teachings of the above application(s) are incorporated herein by reference.

### GOVERNMENT SUPPORT

This invention was made with government support under Grant Numbers DA009158 and DA045882 awarded by the National Institutes of Health. The Government has certain rights in the invention.

### FIELD

The present disclosure relates to compounds, their preparation, and method of use in treatment and prevention of diseases or conditions in which modulation of an endocannabinoid system is beneficial. The disclosure is also concerned with pharmaceutically acceptable compositions comprising compounds of the present disclosure and methods of using said compositions in the treatment of various disorders, diseases, or conditions, in which modulation of the endocannabinoid system is beneficial.

### BACKGROUND

The endocannabinoid system consists of a network of lipid mediators and includes two G protein-coupled receptors (GPCRs), namely cannabinoid receptor type 1 (CB1) and type 2 (CB2), their endogenous ligands (endocannabinoids), namely 2-arachidonoyl glycerol (2-AG) and N-arachidonoylethanolamine [AEA (anandamide)], and related metabolic enzymes. CB1 is highly expressed in the central nervous system (CNS) including the brainstem, hypothalamus, and the reward centers of humans and rodents. CB1 is also expressed (in functionally important levels) in peripheral tissues including myocardium, vascular endothelial and smooth muscle cells, peripheral sensory nerve terminals, hepatic and pancreatic cells, adipose tissue, and kidneys. There is recognition that anatomical locations of CB1 receptors correspond to many known behavioral and physiological effects of CB1 receptor agonists and CB1 inverse agonists. Direct activation of CB1 using orthosteric agonists has been shown to hold promise for the treatment or prevention of neurological, psychiatric, and eating disorders, neuropathic pain, as well as glaucoma, while direct inactivation of the CB1 has been useful to treat substance use disorders and has been strongly associated with the modulation of food intake and related metabolic processes. However, CB1 orthosteric agonists have been associated with inducing psychotropic effects, tolerance associated with their chronic use, and dependence. Direct blockade of the central CB1 receptors and specifically CB1 inverse agonists have been associated with certain gastrointestinal, nervous system, and psychiatric events that occurred in a subset of patients.

CB2 receptors are expressed primarily in cells related to the immune system, and to a lesser extent in the CNS, and have emerged as an attractive therapeutic target for immunomodulation, the treatment of inflammatory and neuropathic pain, neuroinflammation, and neurodegenerative disorders. For certain disease states and within specific tissue, both receptors are expressed and undergo opposing changes wherein CB1 is upregulated while CB2 is downregulated.

It is acknowledged that many GPCRs contain allosteric sites that are distinct from the orthosteric binding pocket of the endogenous agonist and can bind endogenous and/or exogenous ligands. The binding of a ligand at an allosteric site causes a conformational change of the receptor that alters the effects (potency and/or efficacy) of the endogenous agonist (Christopoulos, et al., Nat Rev Drug Discov, 1 (3), 198-210, 2002). Allosteric modulators are categorized as positive or negative depending on whether they enhance or inhibit the signaling of the endogenous ligand. The most prominent therapeutic advantages of an allosteric modulator when compared to an orthosteric ligand are the spatiotemporal control of receptor activation, subtype specificity, and ceiling effect of its activity (Wootten, et al., Nat Rev Drug Discov, 12 (8), 630-644, 2013).

CB1 positive allosteric modulators (PAMS) have shown some promise in treating neurological, psychiatric, and eating disorders, neuropathic pain, glaucoma, post-traumatic stress disorder, and traumatic brain injury. Evidence for the therapeutic utility of CB1 PAMs has been provided by the dose-dependent efficacy in animal models of inflammatory and neuropathic pain without eliciting psychoactive effects and/or considerable tolerance (Slivicky, et al., Biol Psychiatry, 84 (10), 722-733, 2018; Ignatowska-Jankowska, et al., Neuropsychopharmacology, 40 (13), 2948-2959, 2015). The utility of CB1 negative allosteric modulators (NAMs) on the other hand has been explored in models of substance use disorders, food intake and obesity.

In addition to CB1 PAMs or NAMs, an alternative approach to the use of indirect exogenous CB1 agonists or antagonists for medicinal purposes may lie in modulating the activity of endogenous CB1 receptor ligands, for example, the AEA and 2-AG. Although AEA and 2-AG are highly labile, complicating their in vivo evaluation, each has been reported to produce CB1-mediated behavioral effects. For instance, in addition to the CB1-mediated actions of exogenous AEA and 2-AG, their effects also have been examined indirectly by inhibiting their rapid enzymatic degradation in vivo. The most prominent enzymes of the endocannabinoid system are fatty acid amide hydrolase (FAAH), which hydrolyzes anandamide and monoacylglycerol lipase (MGL) which hydrolyzes 2-AG. Their actions control the bioavailability of the endocannabinoids to activate the cannabinoid receptors. Therefore, one hypothesis is that modulation of these enzymes will control the magnitude of the endocannabinoid levels and the duration of their effects on the cannabinoid receptors. Compounds that inhibit the activity of FAAH or MGL can increase the concentrations of, respectively, AEA and 2-AG at CB1 receptors and, after single or combined administration, have been shown to produce varying levels of effect in the CB1 tetrad test.

Dual FAAH/MGL inhibition was found to produce full effects on all measures (antinociception, hypomotility, hypothermia, catalepsy), while selective inhibition of either FAAH or MGL has been reported to produce antinociception in rodent models of acute and/or chronic pain. FAAH inhibitors alone have been reported to modulate addiction-related behavior and, in assays of emotional control, mood and post-traumatic stress, have produced results supporting their further development as anxiolytic and antidepressant drugs. As an alternate strategy to modulate CB1 receptors, MGL inhibitors alone can also be pursued as potential medications by enhancing the endogenous levels of 2-AG. MGL is the primary hydrolytic enzyme of 2-AG and accounts for about 85% of its degradation. While persistent and complete inhibition of MGL for extended periods may be associated with CB1 receptor desensitization, tolerance and hypomotility, such deleterious effects may not be observed with partial MGL blockage, while also limiting the psychotropic effects associated with direct CB1 activation. In addition to MGL, another lipid hydrolase α/β-hydrolase domain containing 6 (ABHD6) has also been found to break down between 4-40% of 2-AG. While MGL is a major player in deactivating endocannabinoid signaling by hydrolyzing presynaptic 2-AG not bound to cannabinoid CB1 receptor, ABHD6, located postsynaptically, has been shown to control separate pools of 2-AG and, thus, might have a different therapeutic profile. Inactivation of ABHD6 by inhibition or disruption of ABHD6 protein expression may also cause a moderate increase in 2-AG levels without CB1-related side effects.

### SUMMARY

It has now been found that compounds described herein and pharmaceutically acceptable compositions thereof, are effective modulators of the endocannabinoid system. Such compounds have general formula I or II:

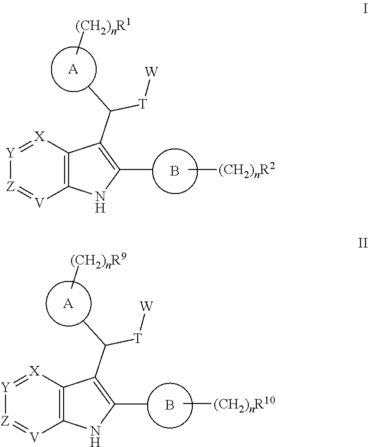

or a tautomer, a stereoisomer, or a pharmaceutically acceptable salt, wherein each of R¹, R², R⁹, R¹⁰, T, W, X, Y, Z, V, n, Ring A, and Ring B is as defined and described herein.

Compounds described herein, and pharmaceutically acceptable compositions thereof, are useful for treating a variety of diseases, disorders, and conditions in which modulation of endocannabinoid system is beneficial. Such diseases, disorders, and conditions include those described herein.

Compounds described herein have the ability to indirectly and selectively modulate (e.g. activate) one or more GPCRs of interest, while simultaneously engaging more than one protein (e.g., CB1 receptor and MGL/FAAH/ABHD6) and/or non-canonical pathways. Additionally, the novel compounds also exhibit higher polarity rendering enhanced aqueous solubility. It is expected that such compounds will display better efficacies in relevant disease models, with less toxicity and fewer side effects.

The following are example features of the compounds of the present disclosure:

They can indirectly and selectively modulate (e.g. activate) a single GPCR of interest without displaying the side effects that are typically associated with direct activation; and/or

They can indirectly and selectively modulate (e.g. activate) a single GPCR of interest while simultaneously engaging more than one protein (e.g., CB1 receptor and MGL/FAAH/ABHD6) and/or non-canonical pathways of interest without displaying the side effects that are typically associated with direct activation or inactivation; and/or

They engage less well-conserved regulatory motifs outside the orthosteric pocket and exert pathway-specific effects on receptor signaling.

They exhibit higher polarity, rendering enhanced aqueous solubility.

It is expected that the compounds of the present disclosure will display better efficacies in relevant disease models, with less toxicity and fewer side effects.

The broad distribution of the endocannabinoid system and its involvement in many pathological conditions makes it an attractive therapeutic target. Compounds of the present disclosure could be used as therapeutics for:
Neuropathic pain, inflammatory pain, acute pain, chronic pain, and other disorders that involve pain; and/or
Neurodegenerative disorders such as Huntington's chorea, Alzheimer's, Parkinson's; and/or
Post-traumatic stress disorder and traumatic brain injury; and/or
Substance use disorders (e.g., cannabinoid, opioid, alcohol, nicotine, etc.).

Additionally, the compounds could be used as novel probes for exploring the therapeutic effects of endocannabinoid system in vitro and in vivo.

### BRIEF DESCRIPTION OF THE DRAWINGS

The foregoing will be apparent from the following more particular description of example embodiments. The drawings are not necessarily to scale, emphasis instead being placed upon illustrating embodiments.

FIGS. 1A-1G represent the concentration-response plots of exemplified compounds for hMGL inhibition.

FIGS. 2A-2H represent concentration-dependent curves of inhibition of forskolin-stimulated CAMP accumulation at CB1 by 2-AG alone or 2-AG in the presence of the exemplified compounds.

### DETAILED DESCRIPTION

A description of example embodiments follows.

In certain aspects, provided herein are modulators of the endocannabinoid system. In some embodiments, such compounds include those of the formulae described herein, or a pharmaceutically acceptable salt, or a stereoisomer, or an isotope-labelled isomer thereof, wherein each variable is as defined and described herein.

Allosteric modulators can alter affinity, potency, and efficacy of orthosteric ligands. Positive allosteric modulators (PAMs) improve the activity of orthosteric ligands and negative allosteric modulators (NAMs) reduce the activity of orthosteric ligands. Both PAMs and NAMs are known to enhance or inhibit the affinity and/or efficacy of an orthosteric ligand respectively without directly activating or inhibiting the receptor on their own. Noteworthy enough, the effects of these various classes of allosteric modulators may depend on the orthosteric ligand, a property referred to as ‘probe dependence’ (Livingston and Traynor, Br J Pharmacol, 175 (14): 2846-2856, 2018). In addition to PAMs and NAMs, silent allosteric modulators (SAMs) may occupy the allosteric site and block the effects of both PAMs and NAMs without showing any activity on their own. To add, a newer class of ligands termed as biased allosteric modulators (BAMs) may exhibit spatial, temporal, and signal pathway specificity, while offering new strategies for designing better, more selective allosteric ligands (Slosky et. al., Trends Pharmacol Sci, 42 (4): 283-299, 2021). The design of dualsteric/bitopic ligands that can target both the orthosteric and allosteric sites of CB1/CB2 simultaneously and the design of allosteric modulators of CB2 are upcoming components in the field.

Known CB1 allosteric modulators lack the potency and/or efficacy and display sub-optimal physicochemical properties. The prominent ligands reported to date do not display the optimal affinities, selectivities, “drug-like” properties (including but not limited to high aqueous solubility and/or metabolic stability), predictable and tunable durations of action, and/or improved therapeutic indexes that are necessary for clinical use without the unwanted undesirable effects.

Overall, compounds that can indirectly and selectively modulate one or more GPCRs of interest, while implicating the endocannabinoid system and engaging more than one protein (e.g., CB1 receptor and MGL/FAAH/ABHD6) and/or non-canonical pathways of interest should be beneficial in the treatment and prevention of various diseases or conditions.

### DEFINITIONS

The following terms are used throughout as defined below.

As used herein, singular articles such as “a”, “an”, “the”, and similar referents in the context of describing the elements (especially in the context of the appended claims) are to be understood to encompass both the singular and the plural, unless otherwise indicated herein or clearly contradicted by context herein. Recitations of ranges of values used herein are merely intended to serve as a shorthand method of referring individually to each separate value falling within range, unless otherwise indicated herein, and each separate value is incorporated into the specification as if it were individually recited herein. As will be understood by one skilled in the art, for any and all purposes, particularly in terms of providing a written description, all ranges disclosed herein, also encompass any and all possible subranges and combinations of subranges thereof. Any listed range can be easily recognized as sufficiently describing and enabling the same range being broken down into at least equal halves, thirds, quarters, fifths, tenths, etc. Also, as will be understood by one skilled in the art a range includes any individual number. For example, a group having 1-6 atoms refers to a group having 1, 2, 3, 4, 5, or 6 atoms. Similarly, a group having 0-4 heteroatoms refers to a group having 0, 1, 2, 3, or 4 heteroatoms.

All methods described herein may be performed in any suitable order unless otherwise indicated herein or otherwise clearly contradicted by context. As used herein, any and all examples, or exemplary language (e.g., “such as”, “for example”, “e.g.,”) are intended merely to better illuminate the embodiments and not pose a limitation on the scope of the claims unless otherwise stated. The materials, methods, and examples used herein, are illustrative and not intended to be limiting. No language in the specification should be interpreted as indicating any non-claimed element is essential.

As used herein, “about” will be understood by a person of ordinary skill in the art and will vary to some extent depending upon the context in which it is used. If there are uses of the term which are not clear to persons of ordinary skill in the art, given the context in which it is used, “about” will mean up to plus or minus 10% of the particular term.

Compounds described herein encompass those described generally above, and are further illustrated by the classes, subclasses, and species disclosed herein. As used herein, the following definitions shall apply unless otherwise indicated. For the purposes of this disclosure, the chemical elements are identified in accordance with the Periodic Table of the Elements, CAS version, Handbook of Chemistry and Physics, 75th Ed. Moreover, general principles of organic chemistry are described in “Organic Chemistry” Thomas Sorrell, University Science Books, Sausalito: 1999, and “March's Advanced Organic Chemistry”, 5th Ed., Ed.: Smith, M. B. and March, J., John Wiley & Sons, New York: 2001, the entire contents of which are hereby incorporated by reference.

The term “aliphatic” or “aliphatic group”, as used herein, means a straight (i.e., unbranched) or branched, substituted or unsubstituted, hydrocarbon chain that is completely saturated or that contains one or more units of unsaturation, or a monocyclic hydrocarbon, or bicyclic hydrocarbon that are completely saturated or that contain one or more units of unsaturation, but which are not aromatic (also referred herein as “carbocycle”, “carboaliphatic”, “carbocyclyl”, or “cycloalkyl”), that has a single or a double point of attachment to the rest of the molecule. Unless otherwise specified, aliphatic groups contain 1-10 aliphatic carbon atoms. In some embodiments, aliphatic groups contain 1-6 aliphatic carbon atoms. In other embodiments, aliphatic groups contain 1-5 aliphatic carbon atoms. In other embodiments, aliphatic groups contain 1-4 aliphatic carbon atoms. In other embodiments, aliphatic groups contain 1-3 aliphatic carbon atoms, and in other embodiments, aliphatic groups contain 1-2 aliphatic carbon atoms. In some embodiments, “cycloaliphatic”, “carbocycle”, “carbocyclyl”, or “cycloalkyl” refer to monocyclic C₃-C₈ hydrocarbons that are completely saturated or that contain one or more units of unsaturation, but which are not aromatic, that have a single or a double point of attachment to the rest of the molecule. Exemplary aliphatic groups are, without limitation, linear or branched, substituted or unsubstituted, C₁-C₈ alkyl, C₂-C₈ alkenyl, C₂-C₈ alkynyl, C₃₋₈ cycloalkyl, C₃₋₈ cycloalkenyl groups and combinations thereof, such as (cycloalkyl)alkyl, (cycloalkenyl)alkyl or (cycloalkyl)alkenyl.

The term “alkyl”, as used herein, refers to C1-10 straight or branched alkyl group. Exemplary alkyl groups are, without limitation, methyl (—CH₃), ethyl (—CH₂CH₃), propyl, isopropyl, butyl, isobutyl, sec-butyl, tert-butyl, homologs, and isomers of n-pentyl, n-hexyl, n-heptyl, n-octyl, and the like. The term “alkenyl”, as used herein, refers to C2-10 straight (i.e., unbranched) or branched hydrocarbon chain containing at least one unit of unsaturation which is a double bond between two carbon atoms, that has a single or a double point of attachment to the rest of the molecule. In some embodiments, the alkenyl group contains one, two or three carbon-carbon double bonds. In some embodiments, alkenyl groups contain 2-6 carbon atoms. In other embodiments, alkenyl groups contain 2-4 carbon atoms, and in other embodiments alkenyl groups contain 2-3 carbon atoms. Exemplary alkenyl groups are, without limitation, vinyl, allyl. The term “alkynyl”, as used herein, refers to C2-10 straight (i.e., unbranched) or branched hydrocarbon chain containing at least one unit of unsaturation which is a triple bond between two carbon atoms, that has a single or a double point of attachment to the rest of the molecule. In some embodiments, the alkenyl group contains one, two or three carbon-carbon triple bonds. In some embodiments, alkynyl groups contain 2-6 carbon atoms. In other embodiments, alkynyl groups contain 2-4 carbon atoms, and in other embodiments alkynyl groups contain 2-3 carbon atoms. Exemplary alkynyl groups are, without limitation, —C≡CH, —C≡CCH₃, —CH₂C≡CCH₃.

The term “heteroatom” means one or more of oxygen, sulfur, nitrogen, phosphorus, or boron (including, any oxidized form of nitrogen, sulfur (e.g., S(═O), S(═O)₂), or phosphorus (e.g., P(═O)); the quaternized form of any basic nitrogen or; a substitutable nitrogen of a heterocyclic ring, for example N (as in 2,3,4,5-tetrahydropyridinyl), NH (as in piperidinyl) or NR (as in N-substituted piperidinyl)).

The term “unsaturated”, as used herein, means that a moiety has one or more units of unsaturation (e.g., one or more double bonds, triple bonds, or combinations thereof).

The term or suffix “ene”, as used herein, used alone or as a part of a larger moiety as in “alkylene”, alkenylene”, refers to bivalent groups. For example, the term “alkylene” refers to a bivalent alkyl group (e.g., —(CH₂)ₙ—, wherein n is a positive integer preferably from 1 to 6, from 1 to 4, from 1 to 3, from 1 to 2, and from 2 to 3. A substituted alkylene chain refers to a group in which one or more methylene hydrogen atoms are replaced with a substituent. Suitable substituents include those described below for a substituted aliphatic group.

The terms “halogen” or “halo”, as used herein, mean fluoride (F), chloride (C1), bromide (Br), iodide (I).

The term “aryl” used alone or as a part of a larger moiety as in “arylalkyl” or “arylalkoxy” refers to monocyclic or bicyclic ring systems having a total of 5-14 ring members, wherein at least one ring in the system is aromatic and wherein each ring in the system contains 3-7 ring members. The term “aryl” is used interchangeably with the term “aryl ring”. In certain embodiments, “aryl” refers to an aromatic ring system. Exemplary aryl groups are, without limitations, phenyl, biphenyl, naphthyl, anthracyl and the like, which optionally include one or more substituents. Also included within the scope of the term “aryl”, as used herein, is a group in which an aromatic ring is fused to one or more nonaromatic rings, such as indanyl, phthalimidyl, naphthimidyl, phenanthridinyl, or tetrahydronapthyl, and the like.

The terms “heteroaryl” and “heteroar”, as used herein, alone or as part of a larger moiety, e.g., “heteroaralkoxy”, refer to groups having 5-10 ring atoms, preferably 5, 6, 9, or 10 ring atoms; having 6, 10, or 14 π electrons shared in a cyclic array; and having, in addition to carbon atoms, from 1 to 5 heteroatoms. Exemplary heteroaryl groups include, without limitations, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, and naphthyridinyl. The terms “heteroaryl” and “heteroar”, as used herein, also include groups in which a heteroaromatic ring is fused to one or more aryl, cycloaliphatic, or heterocyclic rings, where the radical or point of attachment is on the heteroaromatic ring, such as indolyl, isoindolyl, indazolyl, benzimidazolyl, benzofuranyl, dibenzofuranyl, benzothienyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3 (4H)-one, and the like. A heteroaryl group is optionally monocyclic or bicyclic. The term “heteroaryl” is used interchangeably with the terms “heteroaryl ring”, “heteroaryl group”, or “heteroaromatic”, any of which terms include such rings that are optionally substituted.

As used herein, the terms “heterocycle”, “heterocyclic”, “heterocyclyl”, “heterocyclic radical”, and “heterocyclic ring” are used interchangeably and refer to a stable 5- to 7-membered monocyclic or 7- to 10-membered bicyclic heterocyclic moiety that is either saturated or partially unsaturated, but which are not aromatic, and having, in addition to carbon atoms, one or more, preferably 1-4, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur, boron, phosphorus, or nitrogen, the nitrogen is N (as in 2,3,4,5-tetrahydropyridinyl), NH (as in piperidinyl) or NR (as in N-substituted piperidinyl).

A heterocyclic ring can be attached to its pendant group at any heteroatom or carbon atom that results in a stable structure and any of the ring atoms can be optionally substituted. Exemplary saturated or partially unsaturated heterocyclic radicals include, without limitations, tetrahydrofuranyl, tetrahydrothiophenyl, pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl, and the like. The terms “heterocycle”, “heterocyclyl”, “heterocyclyl ring”, “heterocyclic group”, “heterocyclic moiety”, “heterocyclic ring”, and “heterocyclic radical”, are used interchangeably herein, and also include groups in which a heterocyclic ring is fused to one or more aryl, heteroaryl, or cycloaliphatic rings, such as indolinyl, 3H-indolyl, chromanyl, phenanthridinyl, or tetrahydroquinolinyl, and the like, where the radical or point of attachment is on the heterocyclic ring. A heterocyclic group is optionally monocyclic or bicyclic.

As used herein, the term “partially unsaturated” refers to a ring moiety that includes at least one point of unsaturation which can be double or triple bond or any combinations thereof. The term “partially unsaturated” is intended to encompass rings having multiple sites of unsaturation but is not intended to include aryl or heteroaryl moieties, as herein defined.

As described herein, certain compounds of the present disclosure contain “optionally substituted” moieties. In general, the term “substituted”, whether preceded by the term “optionally” or not, refers to a group(s) (e.g., aliphatic, aryl, heteroaryl, heterocyclic group(s)) in which one or more hydrogens of the designated moiety are replaced with a non-hydrogen atom. “Substituted” applies to one or more hydrogens that are either explicit or implicit from the structure (e.g.,

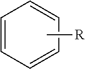

refers to at least

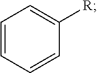

and

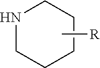

refers to at least

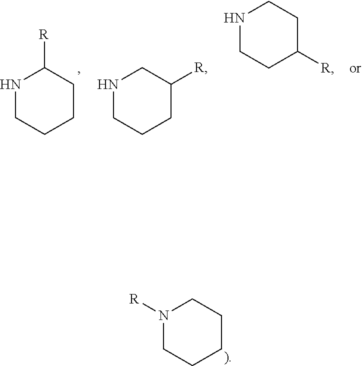

Unless otherwise indicated, an “optionally substituted” group has a suitable substituent at each substitutable position of the group, and when more than one position in any given structure is substituted with more than one substituent(s) selected from a specified group(s), the substituent(s) is/are either the same or different at every position. Combinations of substituents encompassed by the present disclosure are preferably those that results in the formation of stable or chemically feasible compound(s). The term “stable”, as used herein, refers to compounds that are not substantially altered when subjected to conditions to allow for the production, detection, and in certain embodiments, their recovery, purification, and use for one or more of the purposes disclosed herein.

Suitable monovalent substituents on a substitutable carbon atom of an “optionally substituted” group are independently deuterium; halogen; —(CH₂)₀₋₄R^{●}; —(CH₂)₀₋₄OR^{●}; —O—(CH₂)₀₋₄R^{●}; —O—(CH₂)₀₋₄C(═O) OR^{●}; —(CH₂)₀₋₄CH(OR^{●})₂; —(CH₂)₀₋₄SR^{●}; —(CH₂)₀₋₄Ph, which is optionally substituted with R^{●}; —(CH₂)₀₋₄O(CH₂)₀₋₁Ph, which is optionally substituted with R^{●}; —CH₂—CHPh, which is optionally substituted with R^{●}; —(CH₂)₀₋₄O(CH₂)₀₋₁-pyridyl, which is optionally substituted with R^{●}; —NO₂; —C≡N; —N₃; —NCS; —ONO₂; —S(═O)₂F; —OS(═O)₂F; —B(OH)₂; —B(OR^{●})₂; —(CH₂)₀₋₄N(R^{●})₂; —(CH₂)₀₋₄N(R^{●})C(═O) R^{●}; —N(R^{●})C(═S) R^{●}; (CH₂)₀₋₄N(R^{●})C(═O)N(R^{●})₂; —N(R^{●})C(═S)N(R^{●})₂; —(CH₂)₀₋₄N(R^{●})C(═O)OR^{●}; —N(R^{●})N(R^{●})C(═O)R^{●}; —N(R^{●})N(R^{●})C(═O)N(R^{●})₂; —N(R^{●})N(R^{●})C(═O) OR^{●}; —(CH₂)₀₋₄ C(═O)R^{●}; —(CH₂)₀₋₄C(═O) OR^{●}; —C(═S)R^{●}; —(CH₂)₀₋₄C(═O)SR^{●}; —OC(═O)CH₂)₀₋₄SR^{●}; —SC(═S)SR^{●}; —(CH₂)₀₋₄SC(═O)(R^{●}); —(CH₂)₀₋₄C(═O)N(R^{●})₂; —C(═S)N(R^{●})₂; —C(═S)SR^{●}; —(CH₂)₀₋₄C(═O)N(R^{●})₂; —C(═O)N(OR^{●})R^{●}; —C(═O)C(═O)R^{●}; —C(═O) (CH₂)₀₋₄C(═O) R^{●}; —C(NOR^{●})R^{●}; —(CH₂)₀₋₄SSR^{●}; —(CH₂)₀₋₄S(═O)₂R^{●}; —(CH₂)₀₋₄S(═O) R^{●}; —(CH₂)₀₋₄S(═O)₂OR^{●}; —(CH₂)₀₋₄OS(═O)₂R^{●}); —S(═O)₂N(R^{●})₂; —NR^{●}S(═O)₂N(R^{●})₂; —NR^{●}S(═O)₂R^{●}; —N(OR^{●})R^{●}; —CNHN(R^{●})₂; —P(═O)₂R^{●}; —P(═O)(R^{●})₂; —OP(═O)(OR^{●})₂; —OP(═O)(R^{●})₂; —Si(R^{●})₃; —(C₁₋₄ straight or branched)alkylene)O—N(R^{●})₂; —(C₁₋₄ straight or branched alkylene)O—N═(CH₂)₁₋₄ R^{●}; or —(C₁₋₄ straight or branched)alkylene)C(═O)O—N(R^{●})₂, wherein each R^{●} is optionally substituted as defined below and is independently hydrogen, C₁₋₆ aliphatic, —CH₂Ph, —O(CH₂)₀₋₁Ph, —CH₂ (5-6-membered heteroaryl ring), or a 5-6-membered saturated, partially unsaturated, or aryl ring; each of which having 0-4 heteroatoms independently selected from nitrogen, oxygen, sulfur, phosphorus, boron, or notwithstanding the definition above, two independent occurrences of R^{●}, taken together with their intervening atom(s), form a 3-12-membered saturated, partially unsaturated, or aryl mono- or bicyclic ring having 0-4 heteroatoms independently selected from nitrogen, oxygen, sulfur, phosphorus, or boron, which is optionally substituted as defined below.

Suitable monovalent substituents on R^{●}(or the ring formed by taking two independent occurrences of R^{●} together with their intervening atoms), are independently deuterium, halogen, —(CH₂)₀₋₂R^{◯}, -(halo-R^{◯}), —(CH₂)₀₋₂OH, —(CH₂)₀₋₂OR^{◯}, —(CH₂)₀₋₂ CH(OR^{◯})₂, —O(halo-R^{◯}), —NO₂, —C≡N, —N₃, —NCS, —ONO₂, —S(═O)₂F, —OS(═O)₂F, —B(OH)₂, —B(OR^{◯})₂, —(CH₂)₀₋₂C(═O)R^{◯}, —(CH₂)₀₋₂C(═O)OH, —(CH₂)₀₋₂C(═O)OR^{◯}, —(CH₂)₀₋₂ SR^{◯}, —(CH₂)₀₋₂SH, —(CH₂)₀₋₂NH₂, —(CH₂)₀₋₂NHR^{◯}, —(CH₂)₀₋₂N(R^{◯})₂, —Si(R^{◯})₃, —OSi(R^{◯})₃, —C(═O)SR^{◯}, —(C₁₋₄ straight or branched alkylene)C(═O)OR^{◯}, or —SSR^{●} wherein each R^{●} is unsubstituted or where preceded by “halo-” is substituted only with one or more halogens and is independently selected from C₁₋₄ aliphatic, —CH₂Ph, —O(CH₂)₀₋₁Ph, or a 5-6-membered saturated, partially unsaturated, or aryl ring; each of which having 0-4 heteroatoms independently selected from nitrogen, oxygen, sulfur, phosphorus, or boron. Suitable divalent substituents on a saturated carbon atom of R^{●} include ═O or ═S.

Suitable divalent substituents on a saturated carbon atom of an “optionally substituted” group include the following: ═O, ═S, ═NN(R^{†})₂, ═NNHC(═O)R^{†}, ═NNHS(═O)₂R^{†}, ═NR^{†}, ═NOR^{†}, —O(C((R^{†})₂))₂₋₃O—, or —S(C((R^{†})₂))₂₋₃S—, wherein each independent occurrence of R^{†}is selected from hydrogen, C₁₋₆ aliphatic which is substituted as defined below, or an unsubstituted 5-6-membered saturated, partially unsaturated, or aryl ring; each of which having 0-4 heteroatoms independently selected from nitrogen, oxygen, sulfur, phosphorus, or boron. Suitable divalent substituents that are bound to vicinal substitutable carbons of an “optionally substituted” group include: —O(C((R^{†})₂))₂₋₃O—, wherein each independent occurrence of R^{t} is selected from hydrogen, C₁₋₆ aliphatic which is substituted as defined below, or an unsubstituted 5-6-membered saturated, partially unsaturated, or aryl ring; each of which having 0-4 heteroatoms independently selected from nitrogen, oxygen, sulfur, phosphorus, or boron.

Suitable substituents on the aliphatic group of R^{†} include halogen, —R^{†}, -halo —R^{◯}, —OH, —OR^{◯}, —O(halo-R^{◯}), —C≡N, —NO₂, —NCS, —N₃, —B(OH)₂, —ONO₂, —S(═O)₂F, —OS(═O)₂F, —C(═O)OH, —C(═O) OR^{◯}, —NH₂, —NHR^{◯}, or —N(R^{◯})₂ wherein each R^{●} is unsubstituted or where preceded by “halo-” is substituted only with one or more halogens, and is independently C₁₋₄ aliphatic, —CH₂Ph, —O(CH₂)₀₋₁Ph, or a 5-6-membered saturated, partially unsaturated, or aryl ring; each of which having 0-4 heteroatoms independently selected from nitrogen, oxygen, sulfur, phosphorus, or boron.

Suitable substituents on a substitutable nitrogen of an “optionally substituted” group include —R*, —N(R*)₂, —C(═O)R*, —C(═O)OR*, —C(═O)C(═O)R*, —C(═O)CH₂C(═O)R*, —S(═O)₂R*, —S(═O)₂N(R*)₂, —C(═S)NR*, —C(═O)NR*, —C(═NH)NR* or —N(R*)S(═O)₂R*, wherein each R* is independently hydrogen, deuterium, C₁₋₆ aliphatic which is substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R*, taken together with their intervening atom(s) form an unsubstituted 3-12-membered saturated, partially saturated, or aryl monocyclic or bicyclic ring; each of which having 0-4 heteroatoms independently selected from nitrogen, oxygen, sulfur, phosphorus, or boron.

Suitable substituents on the aliphatic group of R^{†} are independently halogen, —R^{◯}, -halo-R^{◯}, —OH, —OR^{◯}), —O (halo-R^{◯}), —C≡N, —NO₂, —NCS, —N₃, —B(OH)₂, —ONO₂, —S(═O)₂F, —OS(═O)₂F, —C(═O)OH, —C(═O)OR^{◯}, —NH₂, —NHR^{◯}, or —N(R^{◯})₂ wherein each R^{●} is unsubstituted or where preceded by “halo-” is substituted only with one or more halogens, and is independently C₁₋₄ aliphatic, —CH₂Ph, —O(CH₂)₀₋₁Ph, or a 5-6-membered saturated, partially unsaturated, or aryl ring; each of which having 0-4 heteroatoms independently selected from nitrogen, oxygen, sulfur, phosphorus, or boron.

In certain embodiments, the terms “optionally substituted”, “optionally substituted alkyl”, “optionally substituted alkenyl”, “optionally substituted alkynyl”, “optionally substituted carbocyclic”, “optionally substituted aryl”, “optionally substituted heteroaryl”, “optionally substituted heterocyclic”, and any other optionally substituted group as used herein, refer to groups that are substituted or unsubstituted by independent replacements or one, two, or three, or more of the hydrogen atoms thereon with typical substituents including, without limitations:
deuterium,
—F, —Cl, —Br, —I,
—OH, protected hydroxy, alkoxy, oxo, thioxo,
—NO₂, —C≡N, —CF₃, —CH₃, —N₃, —NCS, —B(OH)₂, —ONO₂, —S(═O)₂F, —OS(═O)₂F,
—NH₂, protected amino, —NH-alkyl, —NH-alkenyl, —NH-alkynyl, —NH-cycloalkyl, —NH-aryl, —NH-heteroaryl, -dialkylamino, -diarylamino, -diheteroarylamino,
—O-alkyl, —O-alkenyl, —O-alkynyl, —O-cycloalkyl, —O-aryl, —O-heteroaryl, —O-heterocyclyl, —C(═O)-alky, —C(═O)-alkenyl, —C(═O)-alkynyl, —C(═O)-carbocyclyl, —C(═O)-aryl, —C(═O)-heteroaryl, —C(═O)-heterocyclyl,
—C(═O)NH₂, —C(═O)NH-alkyl, —C(═O)NH-alkenyl, —C(═O)NH-alkynyl, —C(═O)NH-carbocyclyl, —C(═O)NH-aryl, —C(═O)NH-heteroaryl, —C(═O)NH-heterocyclyl,
—OC(═O)O-alkyl, —OC(═O)O-alkenyl, —OC(═O)O-alkynyl, —OC(═O)O-carbocyclyl, —OC(═O)O-aryl, —OC(═O)O-heteroaryl, —OC(═O)O-heterocyclyl, —OC(═O)NH₂, —OC(═O)NH-alkyl, —OC(═O)NH-alkenyl, —OC(═O)NH-alkynyl, —OC(═O)NH-carbocyclyl, —OC(═O)NH-aryl, —OC(═O)NH-heteroaryl, —OC(═O)NH-heterocyclyl,
—NHC(═O)-alkyl, —NHC(═O)-alkenyl, —NHC(═O)-alkynyl, —NHC(═O)-carbocyclyl, —NHC(═O)-aryl, —NHC(═O)-heteroaryl, —NHC(═O)-heterocyclyl, —NHC(═O)-haloalkyl, —NHC(═O)O-alkyl, —NHC(═O)O-alkenyl, —NHC(═O)O-alkynyl, —NHC(═O)O-carbocyclyl, —NHC(═O)O-aryl, —NHC(═O)O-heteroaryl, —NHC(═O)O-heterocyclyl, —NHC(═O)NH-alkyl, —NHC(═O)NH-alkenyl, —NHC(═O)NH-alkynyl, —NHC(═O)NH-carbocyclyl, NHC(═O)NH-aryl, —NHC(═O)NH-heteroaryl, —NHC(═O)NH-heterocyclyl, —NHC(═S)NH-alkyl, —NHC(═S)NH-alkenyl, —NHC(═S)NH-alkynyl, —NHC(═S)NH-carbocyclyl, —NHC(═S)NH-aryl, —NHC(═S)NH-heteroaryl, —NHC(═S)NH-heterocyclyl, NHC(═NH)NH-alkyl, —NHC(═NH)NH-alkenyl, —NHC(═NH)NH-alkynyl, NHC(═NH)NH-carbocyclyl, —NHC(═NH)NH-aryl, —NHC(═NH)NH-heteroaryl, —NHC(═NH)NH-heterocyclyl, —NHC(═NH)-alkyl, —NHC(═NH)-alkenyl, —NHC(═NH)-alkynyl, —NHC(═NH)-carbocyclyl, —NHC(═NH)-aryl, —NHC(═NH)-heteroaryl, NHC(═NH)-heterocyclyl,
—S(═O)-alkyl, —S(═O)-alkenyl, —S(═O)-alkynyl, —S(═O)-carbocyclyl, —S(═O)-aryl, —S(═O)-heteroaryl, —S(═O)-heterocyclyl, —S(═O)₂NH₂, —S(═O)₂NH-alkyl, —S(═O)₂NH-alkenyl, —S(═O)₂NH-alkynyl, —S(═O)₂NH-carbocyclyl, —S(═O)₂NH-aryl, —S(═O)₂NH-heteroaryl, —S(═O)₂NH-heterocyclyl, —NHS(═O)₂-alkyl, —NHS(═O)₂-alkenyl, —NHS(═O)₂-alkynyl, —NHS(═O)₂-carbocyclyl, —NHS(═O)₂-aryl, —NHS(═O)₂-heteroaryl, —NHS(═O)₂-heterocyclyl,
CH₂NH₂, —CH₂S(═O)₂CH₃,
-mono-, -di-, or -tri-alkyl silyl, -dialkyl(aryl) silyl,
-alkyl, -alkenyl, -alkynyl, -aryl, -arylalkyl, -heteroaryl, -heteroarylalkyl, -heterocycloalkyl, -cycloalkyl, -carbocyclic, -heterocyclic, -polyalkoxyalkyl, -polyalkoxy, -methoxymethoxy, -methoxyethoxy, —SH, —S-alkyl, —S-alkenyl, —S-alkynyl, —S-carbocyclyl, —S-aryl, —S-heteroaryl, —S-heterocyclyl, or methylthiomethyl.

As used herein, the term “pharmaceutically acceptable salt” refers to those salts which are, within the scope of sound medical judgment, suitable for use in contact with the tissues of humans or lower animals without undue toxicity, irritation, allergic response, and the like, and are commensurate with a reasonable risk/benefit ratio. Pharmaceutically acceptable salts are well known in the art. For example, S. M. Berge et al., describe pharmaceutically acceptable salts in detail in Journal of Pharmaceutical Sciences, 1977, 66, 1-19, incorporated herein by reference. Pharmaceutically acceptable salts of the compounds of the present disclosure include those derived from suitable inorganic and organic acids and bases. Examples of pharmaceutically acceptable, nontoxic acid salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, hexanoate, lactobionate, lactate, laurate, lauryl sulfate, malate, maleate, malonate, methansulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Pharmaceutically acceptable salts derived from appropriate bases include alkali metal, alkaline earth, other metals, ammonium, and N+(C₁₋₄ alkyl)₄ salts. Representative alkali, alkaline, or other metal salts include, without limitations, lithium, sodium, potassium, magnesium, calcium, zinc, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. Exemplary ammonium or amine salts can derive, without limitations, from arginine, lysine, ornithine, dicyclohexylamine, trimethylamine, triethylamine, ethanolamine, diethanolamine, and the like.

Those skilled in the art will appreciate that compounds of the present disclosure may exhibit the phenomena of tautomerism, conformational isomerism, geometric isomerism, and/or stereoisomerism. The formula drawings within the specification and claims of the present disclosure can represent only one of the possible tautomeric, conformational isomeric, geometrical isomeric, and/or stereochemical isomeric forms. Therefore, unless otherwise stated, structures depicted herein are also meant to include all isomeric (e.g., enantiomeric, diastereomeric, and geometric (or conformational)) forms of the structure, for example, the R and S configurations for each asymmetric center, Z and E double bond isomers, and Z and E conformational isomers. Thus, single stereochemical isomers as well as enantiomeric, diastereomeric, and geometric (or conformational) mixtures of the present compounds are within the scope of the present disclosure. Unless otherwise stated, all tautomeric forms of the compounds of the present disclosure are within the scope of the present disclosure.

The term “tautomer”, as used herein, refers to each of two or more isomers of a compound that exist simultaneously in equilibrium and are readily interchangeable by migration of an atom or group within the molecule. The presence and concentration of the isomeric forms will depend on the environment the compound. Tautomers are constitutional isomers of organic compounds that readily interconvert by a chemical reaction called tautomerization. This reaction commonly results in the formal migration of a hydrogen atom or proton, accompanied by a switch of a single bond and adjacent double bond. A nonlimiting example includes 4-methyl imidazole which can exist either in

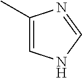

or in

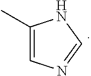

In some embodiments of the present disclosure, the compounds of formula I or II may have an asymmetric center. These compounds exist in enantiomers. Compounds with more than one asymmetric centers may exist as diastereomers. It is to be understood that all such isomers (e.g., enantiomers, diastereomers) and mixtures thereof in any proportion are included within the scope of the present disclosure.

Furthermore, unless otherwise stated, structures described, depicted, or included herein are also meant to encompass compounds that differ only in the presence of one or more isotopically enriched atoms thereof. For a nonlimiting example, compounds having the present structures include the replacement of hydrogen by deuterium or tritium, or the replacement of one or more carbon atom(s) by a ¹¹C—, ¹³C—, or ¹⁴C-enriched carbon atom(s) and are within the scope of the present disclosure. In some embodiments the group comprises one or more deuterium atoms. In other embodiments, the group comprises one or more tritium atoms.

It is moreover intended that a compound of the formula I or II includes isotope-labeled forms thereof. An isotope-labeled form of a compound of the formula I or II is identical to this compound except for the fact that one or more atoms of the compound have been replaced by an atom or atoms having an atomic mass or mass number which differs from the atomic mass or mass number of the atom which usually occurs naturally. Examples of isotopes which are readily commercially available, and which can be incorporated into a compound of the formula I or II by well-known methods include, without limitations, isotopes of hydrogen, carbon, nitrogen, oxygen, phosphorus, fluorine, boron, chlorine, iodine for example ²H, ³H, ¹¹C, ¹³C, ¹⁴C, ¹⁵N, ¹⁷O, ¹⁸O, ³¹P, ³²P, ³⁵S, ¹⁸F, ¹⁰B, ¹¹B, ³⁶Cl, and ¹³¹I. A compound of formula I or II, or a prodrug thereof, or a pharmaceutically acceptable salt of either, which contains one or more of the above-mentioned isotope(s) and/or other isotope(s) of other atom(s) is intended to be encompassed in the present disclosure. An isotope-labeled compound of the formula I or II can be used in a number of beneficial ways. For example, an isotope-labeled compound of the formula I or II into which, for example, an isotope, such as ³H, ¹⁴C, ¹⁸F, or ¹³¹I, has been incorporated, is suitable for medicament and/or tissue distribution assays and/or other binding assays in vitro and in vivo. These radioisotopes (i.e., tritium (³H), carbon-14 (¹⁴C), fluorine-18 (¹⁸F), iodine-131 (¹³¹I)) are particularly preferred owning to simple preparation and excellent detectability. Incorporation of heavier isotopes, for example deuterium (²H), into a compound of the formula I or II has a number of therapeutic advantages owning to the enhanced metabolic stability of this isotope-labeled compound. Enhanced metabolic stability translates directly into an increased in vivo half-life and/or lower dosage(s), which under most circumstances would represent a preferred embodiment of the present disclosure. An isotope-labeled compound of the formula I or II can usually be prepared by carrying out the procedures disclosed in the synthesis schemes and the related description, in the example part and in the preparation part in the present text, by replacing a non-isotope-labeled reacting with readily available isotope-labeled reactant(s). The identification and preparation of any particular isotope-labeled compound is within the skill of person skilled in the art.

A deuterium (²H) atom can be incorporated into a compound of the formula I or II for the purpose to manipulate the oxidative metabolism of the compound by way of the primary kinetic isotope effect. The primary kinetic isotope effect is a change in the rate of a chemical reaction(s) which results from an exchange of isotopic nuclei, which in turn is caused by a change in ground state energies necessary for covalent bond formation after this isotopic exchange. Addition of heavier isotope usually results in a lowering of the ground state energy of a chemical bond and thus causes a reduction in the rate of bond breakage(s). If the bond breakage(s) occurs in or in the vicinity of a saddle point along the coordinate of a multi-product reaction, the product distribution ratios can be altered substantially. For a nonlimiting example, if deuterium is bonded to a carbon atom, rate differences of kM/kD=2-7 are typical. If this rate difference is successfully applied to a compound of the formula I or II that is susceptible to oxidation, the profile of this compound in vivo can be significantly enhanced and result in improved pharmacokinetic properties (e.g., enhanced metabolic stability).

When discovering and developing therapeutic agents, a person skilled in the art is able to optimize pharmacokinetic parameters while retaining desirable pharmacological properties. A person skilled in the art is reasonable to assume that compounds with nonoptimal pharmacokinetic profiles may be susceptible to oxidative metabolism of one or more carbon atom(s). In vitro liver microsomal assays available provide valuable information on the course of such an oxidative metabolism and metabolite profiling permits the identification of such metabolites. In vitro microsomal assays and metabolite profiling, alone or in combination, permit the rational design of compounds of the formula I or II, that contain one or more deuterium atom(s), and have enhanced stability owning to resistance to such oxidative metabolism. Significant improvements in the pharmacokinetic profiles of compounds of the formula I or II are thereby obtained and can be expressed in terms of increases in the in vivo half-life (t_{1/2}), maximal concentration (Cₘₐₓ), area under the curve (AUC) of a dose response plot, bioavailability (% F), reduced in vivo clearance, administered dose, and frequency of dosing regimen.

The following nonlimiting examples are intended to describe the above concept: a compound of the formula I or II which has multiple potential sites of attack for oxidative metabolism, for example benzylic or aromatic hydrogen atom(s), is prepared with various combinations of one or more hydrogen atom(s) replaced by deuterium atom(s). Determinations of the half-life through in vitro microsomal assays enable accurate determination of the extent to which improvement in resistance to oxidative metabolism has improved. In this way, it is determined that the half-life of the compound can be extended by up to 100% as the result of deuterium-hydrogen exchange of this type.

Deuterium-hydrogen exchange in a compound of the formula I or II can also be used to achieve favorable modification of the metabolite spectrum of the starting compound in order to diminish or eliminate the formation of undesired metabolites that may cause toxicity. For a nonlimiting example, if a toxic metabolite arises through oxidative carbon-hydrogen (C—H) bond cleavage(s), it is reasonable for a person skilled in the art to assume that the deuterated analog will greatly diminish or eliminate production of such metabolite. Further information on the state of the art with respect to deuterium-exchange may be found, for example in Hanzlik et al., J. Org. Chem. 55, 3992-3997, 1990, Reider at al., J. Org. Chem. 52, 3326-3334, 1987, Foster, Adv. Drug. Res. 14, 1-40, 1985, Gillette at al., Biochemistry 33 (10), 2927-2937, 1994, and Jarman et al., Carcinogenesis 16 (4), 683-688, 1993.

The compounds of the present disclosure may also exist as solvates, especially hydrates. Hydrates may form during manufacture of the compounds or compositions comprising the compounds, or hydrates may form over time due to the hydroscopic nature of the compounds. Compounds of the present disclosure may exist as organic solvates as well, including, without limitations, dimethylformamide, organic ethers or alcohols, and the like. The identification and preparation of any particular solvate is within the skill of person skilled in the art.

The term “prodrug”, as used herein, refers to a substance that is prepared in an inactive form that is converted to an active form (i.e., drug) within the body or cells thereof by the actions of, for example, endogenous enzymes or other chemicals and/or conditions (e.g., acidic pH, basic pH).

The term “modulator”, as used herein, is defined as a compound that binds to and/or modulates the actions of a target. In some embodiments, a modulator binds on a topologically distinct site when compared to the binding site of an endogenous agonist of the protein target and potentiates the actions of the endogenous agonist on the protein target.

The term “modulate,” as used herein, is defined as a compound or molecule that alters the activity of a target, such as enzymes, genes, and/or other compounds, in a biological pathway. In some embodiments, modulate may refer to activate a target. In some embodiments, modulate may refer to inactivate or inhibit a target. In some embodiments, modulate may refer to upregulate or downregulate a target. In some embodiments, a compound that modulates a target can cause a conformational change in the target.

Combinations of substituents and variables envisioned by this disclosure are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic, or prophylactic administration to a subject).

The recitation of a listing of chemical groups in any definition of a variable herein includes definition of that variable as any single group or combination of listed groups. The recitation of an embodiment for a variable herein includes that embodiment as any single embodiment or in combination with any other embodiments or portions thereof.

### Description of Compounds

According to one aspect, the present disclosure provides a compound of formula I, a pharmaceutically acceptable salt, solvate, hydrate, polymorph, enantiomer, diastereomer, geometric isomer, racemate, tautomer, rotamer, atropisomer, isotopic variation, or N-oxide thereof:

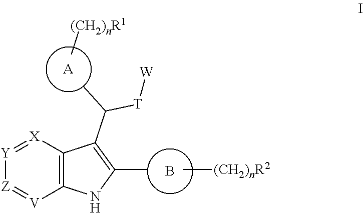

wherein:
   Ring A is C5-10 aryl, a 3-7 membered monocyclic heteroaryl ring having 1-4 heteroatoms independently selected from nitrogen, oxygen, or sulfur, a 3-8 membered saturated or partially unsaturated carbocyclic ring, a 3-7 membered saturated or partially unsaturated heterocyclic ring having 1-4 heteroatoms independently selected from nitrogen, oxygen, phosphorus, boron, or sulfur; each of which is optionally substituted;
   Ring B is C5-10 aryl, a 3-7 membered monocyclic heteroaryl ring having 1-4 heteroatoms independently selected from nitrogen, oxygen, or sulfur, a 3-8 membered saturated or partially unsaturated carbocyclic ring, a 3-7 membered saturated or partially unsaturated heterocyclic ring having 1-4 heteroatoms independently selected from nitrogen, oxygen, phosphorus, boron, or sulfur; each of which is optionally substituted;
   X, Y, Z, V are independently selected from nitrogen, —C((CH₂)ₙR₃)—, or —C(R₄)—;
   R₁, R₂, R₃ and R₄ are independently selected from hydrogen, —B(OH)₂, —B(OR₅)₂, —B(OH)(OR₆), —B(OR₇)₂NR₈, BF₃, provided that at least one of them is —B(OH)₂, —B(OR₅)₂, —B(OH)(OR₆), —B(OR₇)₂NR₈, or BF₃;
   each n is independently selected from 0, 1, 2, 3 or 4; as used in this disclosure when an integer such as n is 0 the structural portion modified by that integer is absent and the adjacent subunits are directly connected;
   each R¹, R², R³ and R⁴ are independently selected from hydrogen, deuterium, F, Cl, Br, I, C≡N, N₃, NCS, —S(═O)₂F, —OS(═O)₂F, —R, —OR, —SR, —C(═O)R, —OC(═O)R, —S(═O)₂R, —S(═O)R, —N(R) S(═O)₂R, —S(═O)₂NR₂, —C(═O) OR, —C(═O)NR₂, —N(R)C(═O)R, —N(R)C(═O)NR₂, —C(═O)N(R)C(═O)R, —NR₂, optionally substituted C5-10 aryl, optionally substituted C5-10 heteroaryl having 1-4 heteroatoms independently selected from nitrogen, oxygen, or sulfur, optionally substituted C₃₋₈ saturated or partially unsaturated carbocyclic ring, optionally substituted C₃₋₈ saturated or partially unsaturated heterocyclic ring having 1-4 heteroatoms independently selected from nitrogen, oxygen, or sulfur;
   R₅ is independently selected from C₁₋₄ alkyl, C₁₋₄ alkenyl, C₁₋₄ alkynyl, C₃-6 cycloalkyl; each of which is optionally substituted; two independent occurrences of R⁵, taken together with their intervening atom(s), form an optionally substituted 4-8-membered saturated or partially unsaturated cyclic ring having 0-4 heteroatoms independently selected from nitrogen, oxygen, boron, or sulfur;
   R⁶ is independently selected from C₁₋₄ alkyl, C₁₋₄ alkenyl, C₁₋₄ alkynyl, C₃-6 cycloalkyl; each of which is optionally substituted; or forms an optionally substituted 4-8 membered saturated or partially saturated heterocyclic ring with an atom of Ring A, Ring B, or the indole core having 0-4 heteroatoms independently selected from nitrogen, oxygen, or boron;
   R⁷ is independently selected from C₁₋₄ alkyl, C₁₋₄ alkenyl, C₁₋₄ alkynyl, C₃-6 cycloalkyl; each of which is optionally substituted; each independent occurrence of R⁷, taken together with its intervening atom(s), boron, and nitrogen, forms an optionally substituted 4-8-membered saturated or partially unsaturated cyclic ring having 0-4 heteroatoms independently selected from nitrogen, oxygen, boron, or sulfur;
   R₈ is C₁₋₄ alkyl, C₁₋₄ alkenyl, C₁₋₄ alkynyl, C₃₋₆ cycloalkyl; each of which is optionally substituted;
   each R is independently hydrogen, C₁₋₆ aliphatic, C₅₋₁₀ aryl, a 3-7 membered monocyclic heteroaryl ring having 1-4 heteroatoms independently selected from nitrogen, oxygen, or sulfur, a 3-8 membered saturated or partially unsaturated carbocyclic ring, a 3-7 membered saturated or partially unsaturated heterocyclic ring having 1-4 heteroatoms independently selected from nitrogen, oxygen, or sulfur; each of which is optionally substituted.
   T is C₁₋₄ alkylene, C₁₋₄ alkenylene, C₁₋₄ alkynylene, C3-6 cycloalkylene; each of which is optionally substituted;
   W is selected from —NO₂, —CF₃, C≡N, NH₂, NHC(═O) CH₂-halogen, —ONO₂, —S(═O)₂F, —OS(═O)₂F, —OH, —C(═O)OH, —B(OH)₂, or —SF₅.

According to another aspect, provided herein is a compound of formula II, a pharmaceutically acceptable salt, solvate, hydrate, polymorph, enantiomer, diastereomer, geometric isomer, racemate, tautomer, rotamer, atropisomer, isotopic variation, or N-oxide thereof:

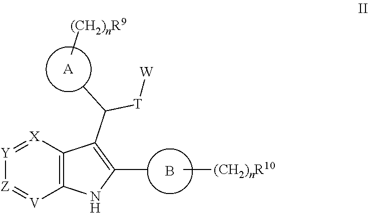

wherein:
   Ring A is C₅₋₁₀ aryl, a 3-7 membered monocyclic heteroaryl ring having 1-4 heteroatoms independently selected from nitrogen, oxygen, or sulfur, a 3-8 membered saturated or partially unsaturated carbocyclic ring, a 3-7 membered saturated or partially unsaturated heterocyclic ring having 1-4 heteroatoms independently selected from nitrogen, oxygen, phosphorus, boron, or sulfur; each of which is optionally substituted;
   Ring B is C5-10 aryl, a 3-7 membered monocyclic heteroaryl ring having 1-4 heteroatoms independently selected from nitrogen, oxygen, or sulfur, a 3-8 membered saturated or partially unsaturated carbocyclic ring, a 3-7 membered saturated or partially unsaturated heterocyclic ring having 1-4 heteroatoms independently selected from nitrogen, oxygen, phosphorus, boron, or sulfur; each of which is optionally substituted;
   X, Y, Z, V are independently selected from nitrogen, —C((CH₂)ₙR₁₁)—, or —C(R₁₂)—;
   R⁹, R¹⁰, R¹¹, and R¹² are independently selected from hydrogen, NCS, S(═O)₂F, NHC(═O)C₂₋₄ alkenyl, NHC(═O) CH₂-halogen, or N₃ provided that at least one of them is —NCS, S(═O)₂F, NHC(═O)C₂₋₄ alkenyl, NHC(═O) CH₂-halogen, or N₃;
   each n is independently selected from 0, 1, 2, 3 or 4; as used in this disclosure when an integer such as n is 0 the structural portion modified by that integer is absent and the adjacent subunits are directly connected;
   each R⁹, R¹⁰, R¹¹, and R¹² are independently selected from hydrogen, deuterium, F, Cl, Br, I, C≡N, N₃, NCS, —S(═O)₂F, —OS(═O)₂F, —R, —OR, —SR, —C(═O)R, —OC(═O)R, —S(═O)₂R, —S(═O)R, —N(R) S(═O)₂R, —S(═O)₂NR₂, —C(═O) OR, —C(═O)NR₂, —N(R)C(═O)R, —N(R)C(═O)NR₂, —C(═O)N(R)C(═O)R, —NR₂, optionally substituted C₅₋₁₀ aryl, optionally substituted C₅₋₁₀ heteroaryl having 1-4 heteroatoms independently selected from nitrogen, oxygen, or sulfur, optionally substituted C₃₋₈ saturated or partially unsaturated carbocyclic ring, optionally substituted C₃₋₈ saturated or partially unsaturated heterocyclic ring having 1-4 heteroatoms independently selected from nitrogen, oxygen, or sulfur;
   each R is independently hydrogen, C₁₋₆ aliphatic, C₅₋₁₀ aryl, a 3-7 membered monocyclic heteroaryl ring having 1-4 heteroatoms independently selected from nitrogen, oxygen, or sulfur, a 3-8 membered saturated or partially unsaturated carbocyclic ring, a 3-7 membered saturated or partially unsaturated heterocyclic ring having 1-4 heteroatoms independently selected from nitrogen, oxygen, or sulfur; each of which is optionally substituted.
   T is C₁₋₄ alkylene, C₁₋₄ alkenylene, C₁₋₄ alkynylene, C₃₋₆ cycloalkylene; each of which is optionally substituted;
   W is selected from —NO₂, —CF₃, —C≡N, —ONO₂, NH₂, NHC(═O) CH₂-halogen, —S(═O)₂F, —OS(═O)₂F, —OH, —C(═O) OH, or —SF₅.

In certain embodiments, Ring A is C₅₋₁₀ aryl, a 3-7 membered monocyclic heteroaryl ring having 1-4 heteroatoms independently selected from nitrogen, oxygen, or sulfur each of which is optionally substituted. Values for the remaining variables are as defined above and described in the first aspect.

In certain embodiments, Ring A is C₅₋₁₀ aryl, which is optionally substituted. Values for the remaining variables are as defined above and described in the first aspect.

In certain embodiments, Ring A is phenyl. Values for the remaining variables are as defined above and described in the first aspect.

In certain embodiments, Ring A is

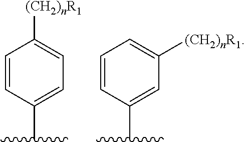

Values for the remaining variables are as defined above and described in the first aspect.

In certain embodiments, Ring A is

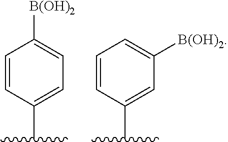

Values for the remaining variables are as defined above and described in the first aspect.

In certain embodiments, Ring A is

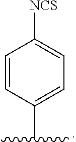

Values for the remaining variables are as defined above and described in the second aspect.

In certain embodiments, Ring A is

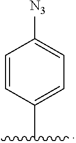 [SMILES: *c1ccc(N=[N+]=[N-])cc1]

Values for the remaining variables are as defined above and described in the second aspect.

In certain embodiments, Ring B is C₅₋₁₀ aryl, a 3-7 membered monocyclic heteroaryl ring having 1-4 heteroatoms independently selected from nitrogen, oxygen, or sulfur each of which is optionally substituted. Values for the remaining variables are as defined above and described in the first aspect.

In certain embodiments, Ring B is C₅₋₁₀ aryl, which is optionally substituted. Values for the remaining variables are as defined above and described in the first aspect.

In certain embodiments, Ring B is phenyl. Values for the remaining variables are as defined above and described in the first aspect.

In certain embodiments, Ring B is

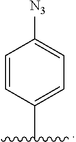 [SMILES: *c1ccc(N=[N+]=[N-])cc1]

Values for the remaining variables are as defined above and described in the first aspect.

In certain embodiments, Ring B is

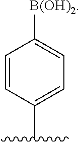 [SMILES: *c1ccc(B(O)O)cc1]

Values for the remaining variables are as defined above and described in the first aspect.

In certain embodiments, Ring B is

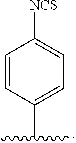

Values for the remaining variables are as defined above and described in the first aspect.

In certain embodiments, Ring B is

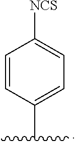

Values for the remaining variables are as defined above and described in the second aspect.

In certain embodiments, Ring B is

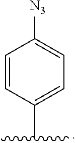 [SMILES: *c1ccc(N=[N+]=[N-])cc1]

Values for the remaining variables are as defined above and described in the second aspect.

In certain embodiments, Ring B is

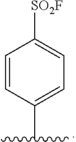 [SMILES: *c1ccc(S(=O)(=O)F)cc1]

Values for the remaining variables are as defined above and described in the second aspect.

In certain embodiments, X, Y, Z, V are —CH—. Values for the remaining variables are as defined above and described in the first aspect.

In certain embodiments, X, Y, Z, V are —CH—. Values for the remaining variables are as defined above and described in the second aspect.

In certain embodiments, at least one of X, Y, Z, V is —C(R⁴)—. Values for the remaining variables are as defined above and described in the first aspect.

In certain embodiments, at least one of X, Y, Z, V is —C(R⁴)—. Values for the remaining variables are as defined above and described in the second aspect.

In certain embodiments, R⁴ is methyl. In certain embodiments, R⁴ is —C≡N.

Values for the remaining variables are as defined above and described in the first aspect.

In certain embodiments, R¹² is methyl. In certain embodiments, R¹² is —C≡N. Values for the remaining variables are as defined above and described in the second aspect.

In certain embodiments, W is —NO₂. Values for the remaining variables are as defined above and described in the first aspect.

In certain embodiments, W is —NO₂. Values for the remaining variables are as defined above and described in the second aspect.

In certain embodiments, T is C₁₋₄ alkylene, C₃₋₆ cycloalkylene; each of which is optionally substituted. Values for the remaining variables are as defined above and described in the first aspect.

In certain embodiments, T is optionally substituted C₁₋₄ alkylene. Values for the remaining variables are as defined above and described in the first aspect.

In certain embodiments, T is —CH₂—. Values for the remaining variables are as defined above and described in the first aspect.

In certain embodiments, provided is a compound of formula Ia.

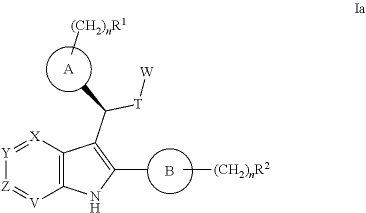

Values for the variables are as defined above and described in the first aspect.

In certain embodiments, provided is a compound of formula Ib.

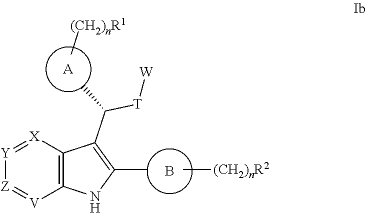

Values for the variables are as defined above and described in the first aspect.

In certain embodiments, provided is a compound of formula IIa.

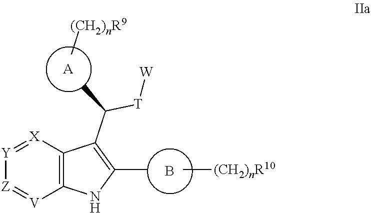

Values for the variables are as defined above and described in the second aspect.

In certain embodiments, provided is a compound of formula IIb.

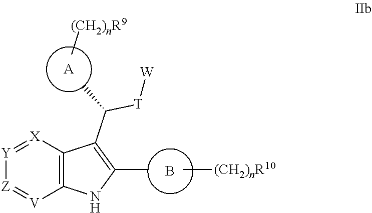

Values for the variables are as defined above and described in the second aspect.

As used in this disclosure when an integer such as n is 0 the structural portion modified by that integer is absent and the adjacent subunits are directly connected. For example, in certain embodiments R₁ is directly connected with Ring A. In other embodiments R¹ is directly connected with Ring B. In some embodiments, R₁ is directly connected with Ring A and Ring A is phenyl. In other embodiments, R¹ is directly connected with Ring B and Ring B is phenyl. Values for the remaining variables are as defined above and described in the first aspect.

In certain embodiments, provided is a compound and their pharmaceutically acceptable salts, solvates, hydrates, polymorphs, enantiomers, diastereomers, geometric isomers, racemates, tautomers, rotamers, atropisomers, isotopic variations, or N-oxides thereof selected from:
3-(2-nitro-1-(4-(4,4,5,5-tetramethyl-1,3,2-dioxaborolan-2-yl)phenyl)ethyl)-2-phenyl-1H-indole
(4-(2-nitro-1-(2-phenyl-1H-indol-3-yl)ethyl)phenyl)boronic acid
3-(2-nitro-1-(3-(4,4,5,5-tetramethyl-1,3,2-dioxaborolan-2-yl)phenyl)ethyl)-2-phenyl-1H-indole
(3-(2-nitro-1-(2-phenyl-1H-indol-3-yl)ethyl)phenyl)boronic acid
(R)-(4-(2-nitro-1-(2-phenyl-1H-indol-3-yl)ethyl)phenyl)boronic acid(S)
(4-(2-nitro-1-(2-phenyl-1H-indol-3-yl)ethyl)phenyl)boronic acid
3-(2-nitro-1-phenylethyl)-2-(4-(4,4,5,5-tetramethyl-1,3,2-dioxaborolan-2-yl)phenyl)-1H-indole
(4-(3-(2-nitro-1-phenylethyl)-1H-indol-2-yl)phenyl)boronic acid
2-(4-azidophenyl)-3-(2-nitro-1-(4-(4,4,5,5-tetramethyl-1,3,2-dioxaborolan-2-yl)phenyl)ethyl)-1H-indole
(4-(1-(2-(4-azidophenyl)-1H-indol-3-yl)-2-nitroethyl)phenyl)boronic acid
(4-(1-(2-(4-isothiocyanatophenyl)-1H-indol-3-yl)-2-nitroethyl)phenyl)boronic acid
3-(2-nitro-1-phenylethyl)-2-phenyl-6-(4,4,5,5-tetramethyl-1,3,2-dioxaborolan-2-yl)-1H-indole
(3-(2-nitro-1-phenylethyl)-2-phenyl-1H-indol-6-yl)boronic acid
(2-(2-nitro-1-(2-phenyl-1H-indol-3-yl)ethyl)phenyl)boronic acid
(3-(2-nitro-1-(2-phenyl-1H-indol-3-yl)ethyl)phenyl)boronic acid
(4-(2-nitro-1-(2-phenyl-1H-indol-3-yl)ethyl)phenyl)boronic acid
(3-(2-nitro-1-phenylethyl)-2-phenyl-1H-indol-4-yl)boronic acid
(3-(2-nitro-1-phenylethyl)-2-phenyl-1H-indol-5-yl)boronic acid
(3-(2-nitro-1-phenylethyl)-2-phenyl-1H-indol-6-yl)boronic acid
(3-(2-nitro-1-phenylethyl)-2-phenyl-1H-indol-7-yl)boronic acid
(2-(3-(2-nitro-1-phenylethyl)-1H-indol-2-yl)phenyl)boronic acid
(3-(3-(2-nitro-1-phenylethyl)-1H-indol-2-yl)phenyl)boronic acid
(4-(3-(2-nitro-1-phenylethyl)-1H-indol-2-yl)phenyl)boronic acid
3-(1-(2-isothiocyanatophenyl)-2-nitroethyl)-2-phenyl-1H-indole
3-(1-(3-isothiocyanatophenyl)-2-nitroethyl)-2-phenyl-1H-indole
3-(1-(4-isothiocyanatophenyl)-2-nitroethyl)-2-phenyl-1H-indole
4-isothiocyanato-3-(2-nitro-1-phenylethyl)-2-phenyl-1H-indole
5-isothiocyanato-3-(2-nitro-1-phenylethyl)-2-phenyl-1H-indole
6-isothiocyanato-3-(2-nitro-1-phenylethyl)-2-phenyl-1H-indole
7-isothiocyanato-3-(2-nitro-1-phenylethyl)-2-phenyl-1H-indole
2-(2-isothiocyanatophenyl)-3-(2-nitro-1-phenylethyl)-1H-indole
2-(3-isothiocyanatophenyl)-3-(2-nitro-1-phenylethyl)-1H-indole
2-(4-isothiocyanatophenyl)-3-(2-nitro-1-phenylethyl)-1H-indole
2-(2-nitro-1-(2-phenyl-1H-indol-3-yl)ethyl)benzenesulfonyl fluoride
3-(2-nitro-1-(2-phenyl-1H-indol-3-yl)ethyl)benzenesulfonyl fluoride
4-(2-nitro-1-(2-phenyl-1H-indol-3-yl)ethyl)benzenesulfonyl fluoride
3-(2-nitro-1-phenylethyl)-2-phenyl-1H-indole-4-sulfonyl fluoride
3-(2-nitro-1-phenylethyl)-2-phenyl-1H-indole-5-sulfonyl fluoride
3-(2-nitro-1-phenylethyl)-2-phenyl-1H-indole-6-sulfonyl fluoride
3-(2-nitro-1-phenylethyl)-2-phenyl-1H-indole-7-sulfonyl fluoride
2-(3-(2-nitro-1-phenylethyl)-1H-indol-2-yl)benzenesulfonyl fluoride
3-(3-(2-nitro-1-phenylethyl)-1H-indol-2-yl)benzenesulfonyl fluoride
4-(3-(2-nitro-1-phenylethyl)-1H-indol-2-yl)benzenesulfonyl fluoride
(5-(2-nitro-1-(2-phenyl-1H-indol-3-yl)ethyl)thiophen-2-yl)boronic acid
(5-(2-nitro-1-(2-phenyl-1H-indol-3-yl)ethyl)thiophen-3-yl)boronic acid
(2-(2-nitro-1-(2-phenyl-1H-indol-3-yl)ethyl)thiophen-3-yl)boronic acid
(3-(2-nitro-1-(thiophen-2-yl)ethyl)-2-phenyl-1H-indol-4-yl)boronic acid
(3-(2-nitro-1-(thiophen-2-yl)ethyl)-2-phenyl-1H-indol-5-yl)boronic acid
(3-(2-nitro-1-(thiophen-2-yl)ethyl)-2-phenyl-1H-indol-6-yl)boronic acid
(3-(2-nitro-1-(thiophen-2-yl)ethyl)-2-phenyl-1H-indol-7-yl)boronic acid
(2-(3-(2-nitro-1-(thiophen-2-yl)ethyl)-1H-indol-2-yl)phenyl)boronic acid
(3-(3-(2-nitro-1-(thiophen-2-yl)ethyl)-1H-indol-2-yl)phenyl)boronic acid
(4-(3-(2-nitro-1-(thiophen-2-yl)ethyl)-1H-indol-2-yl)phenyl)boronic acid
(5-(1-(6-methyl-2-phenyl-1H-indol-3-yl)-2-nitroethyl)thiophen-2-yl)boronic acid
(5-(1-(6-methyl-2-phenyl-1H-indol-3-yl)-2-nitroethyl)thiophen-3-yl)boronic acid
(2-(1-(6-methyl-2-phenyl-1H-indol-3-yl)-2-nitroethyl)thiophen-3-yl)boronic acid
(2-(6-methyl-3-(2-nitro-1-(thiophen-2-yl)ethyl)-1H-indol-2-yl)phenyl)boronic acid
(3-(6-methyl-3-(2-nitro-1-(thiophen-2-yl)ethyl)-1H-indol-2-yl)phenyl)boronic acid
(4-(6-methyl-3-(2-nitro-1-(thiophen-2-yl)ethyl)-1H-indol-2-yl)phenyl)boronic acid
3-(1-(5-isothiocyanatothiophen-2-yl)-2-nitroethyl)-2-phenyl-1H-indole
3-(1-(4-isothiocyanatothiophen-2-yl)-2-nitroethyl)-2-phenyl-1H-indole
3-(1-(3-isothiocyanatothiophen-2-yl)-2-nitroethyl)-2-phenyl-1H-indole
4-isothiocyanato-3-(2-nitro-1-(thiophen-2-yl)ethyl)-2-phenyl-1H-indole
5-isothiocyanato-3-(2-nitro-1-(thiophen-2-yl)ethyl)-2-phenyl-1H-indole
6-isothiocyanato-3-(2-nitro-1-(thiophen-2-yl)ethyl)-2-phenyl-1H-indole
7-isothiocyanato-3-(2-nitro-1-(thiophen-2-yl)ethyl)-2-phenyl-1H-indole
2-(2-isothiocyanatophenyl)-3-(2-nitro-1-(thiophen-2-yl)ethyl)-1H-indole
2-(3-isothiocyanatophenyl)-3-(2-nitro-1-(thiophen-2-yl)ethyl)-1H-indole
2-(4-isothiocyanatophenyl)-3-(2-nitro-1-(thiophen-2-yl)ethyl)-1H-indole
5-(2-nitro-1-(2-phenyl-1H-indol-3-yl)ethyl)thiophene-2-sulfonyl fluoride
5-(2-nitro-1-(2-phenyl-1H-indol-3-yl)ethyl)thiophene-3-sulfonyl fluoride
2-(2-nitro-1-(2-phenyl-1H-indol-3-yl)ethyl)thiophene-3-sulfonyl fluoride
3-(2-nitro-1-(thiophen-2-yl)ethyl)-2-phenyl-1H-indole-4-sulfonyl fluoride
3-(2-nitro-1-(thiophen-2-yl)ethyl)-2-phenyl-1H-indole-5-sulfonyl fluoride
3-(2-nitro-1-(thiophen-2-yl)ethyl)-2-phenyl-1H-indole-6-sulfonyl fluoride
3-(2-nitro-1-(thiophen-2-yl)ethyl)-2-phenyl-1H-indole-7-sulfonyl fluoride
2-(3-(2-nitro-1-(thiophen-2-yl)ethyl)-1H-indol-2-yl)benzenesulfonyl fluoride
3-(3-(2-nitro-1-(thiophen-2-yl)ethyl)-1H-indol-2-yl)benzenesulfonyl fluoride
4-(3-(2-nitro-1-(thiophen-2-yl)ethyl)-1H-indol-2-yl)benzenesulfonyl fluoride
(2-(3,3,3-trifluoro-1-(2-phenyl-1H-indol-3-yl)propyl)phenyl)boronic acid
(3-(3,3,3-trifluoro-1-(2-phenyl-1H-indol-3-yl)propyl)phenyl)boronic acid
(4-(3,3,3-trifluoro-1-(2-phenyl-1H-indol-3-yl)propyl)phenyl)boronic acid
(2-phenyl-3-(3,3,3-trifluoro-1-phenylpropyl)-1H-indol-4-yl)boronic acid
(2-phenyl-3-(3,3,3-trifluoro-1-phenylpropyl)-1H-indol-5-yl)boronic acid
(2-phenyl-3-(3,3,3-trifluoro-1-phenylpropyl)-1H-indol-6-yl)boronic acid
(2-phenyl-3-(3,3,3-trifluoro-1-phenylpropyl)-1H-indol-7-yl)boronic acid
(2-(3-(3,3,3-trifluoro-1-phenylpropyl)-1H-indol-2-yl)phenyl)boronic acid
(3-(3-(3,3,3-trifluoro-1-phenylpropyl)-1H-indol-2-yl)phenyl)boronic acid
(4-(3-(3,3,3-trifluoro-1-phenylpropyl)-1H-indol-2-yl)phenyl)boronic acid
2-phenyl-3-(3,3,3-trifluoro-1-(2-isothiocyanatophenyl)propyl)-1H-indole
2-phenyl-3-(3,3,3-trifluoro-1-(3-isothiocyanatophenyl)propyl)-1H-indole
2-phenyl-3-(3,3,3-trifluoro-1-(4-isothiocyanatophenyl)propyl)-1H-indole
4-isothiocyanato-2-phenyl-3-(3,3,3-trifluoro-1-phenylpropyl)-1H-indole
5-isothiocyanato-2-phenyl-3-(3,3,3-trifluoro-1-phenylpropyl)-1H-indole
6-isothiocyanato-2-phenyl-3-(3,3,3-trifluoro-1-phenylpropyl)-1H-indole
7-isothiocyanato-2-phenyl-3-(3,3,3-trifluoro-1-phenylpropyl)-1H-indole
2-(2-isothiocyanatophenyl)-3-(3,3,3-trifluoro-1-phenylpropyl)-1H-indole
2-(3-isothiocyanatophenyl)-3-(3,3,3-trifluoro-1-phenylpropyl)-1H-indole
2-(4-isothiocyanatophenyl)-3-(3,3,3-trifluoro-1-phenylpropyl)-1H-indole
2-(3,3,3-trifluoro-1-(2-phenyl-1H-indol-3-yl)propyl)benzenesulfonyl fluoride
3-(3,3,3-trifluoro-1-(2-phenyl-1H-indol-3-yl)propyl)benzenesulfonyl fluoride
4-(3,3,3-trifluoro-1-(2-phenyl-1H-indol-3-yl)propyl)benzenesulfonyl fluoride
2-phenyl-3-(3,3,3-trifluoro-1-phenylpropyl)-1H-indole-4-sulfonyl fluoride
2-phenyl-3-(3,3,3-trifluoro-1-phenylpropyl)-1H-indole-5-sulfonyl fluoride
2-phenyl-3-(3,3,3-trifluoro-1-phenylpropyl)-1H-indole-6-sulfonyl fluoride
2-phenyl-3-(3,3,3-trifluoro-1-phenylpropyl)-1H-indole-7-sulfonyl fluoride
2-(3-(3,3,3-trifluoro-1-phenylpropyl)-1H-indol-2-yl)benzenesulfonyl fluoride
3-(3-(3,3,3-trifluoro-1-phenylpropyl)-1H-indol-2-yl)benzenesulfonyl fluoride
4-(3-(3,3,3-trifluoro-1-phenylpropyl)-1H-indol-2-yl)benzenesulfonyl fluoride
(5-(3,3,3-trifluoro-1-(2-phenyl-1H-indol-3-yl)propyl)thiophen-2-yl)boronic acid
(5-(3,3,3-trifluoro-1-(2-phenyl-1H-indol-3-yl)propyl)thiophen-3-yl)boronic acid
(2-(3,3,3-trifluoro-1-(2-phenyl-1H-indol-3-yl)propyl)thiophen-3-yl)boronic acid
(2-phenyl-3-(3,3,3-trifluoro-1-(thiophen-2-yl)propyl)-1H-indol-4-yl)boronic acid
(2-phenyl-3-(3,3,3-trifluoro-1-(thiophen-2-yl)propyl)-1H-indol-5-yl)boronic acid
(2-phenyl-3-(3,3,3-trifluoro-1-(thiophen-2-yl)propyl)-1H-indol-6-yl)boronic acid
(2-phenyl-3-(3,3,3-trifluoro-1-(thiophen-2-yl)propyl)-1H-indol-7-yl)boronic acid
(2-(3-(3,3,3-trifluoro-1-(thiophen-2-yl)propyl)-1H-indol-2-yl)phenyl)boronic acid
(4-(3-(3,3,3-trifluoro-1-(thiophen-2-yl)propyl)-1H-indol-2-yl)phenyl)boronic acid
(3-(3-(3,3,3-trifluoro-1-(thiophen-2-yl)propyl)-1H-indol-2-yl)phenyl)boronic acid
2-phenyl-3-(3,3,3-trifluoro-1-(5-isothiocyanatothiophen-2-yl)propyl)-1H-indole
2-phenyl-3-(3,3,3-trifluoro-1-(4-isothiocyanatothiophen-2-yl)propyl)-1H-indole
2-phenyl-3-(3,3,3-trifluoro-1-(3-isothiocyanatothiophen-2-yl)propyl)-1H-indole
4-isothiocyanato-2-phenyl-3-(3,3,3-trifluoro-1-(thiophen-2-yl)propyl)-1H-indole
5-isothiocyanato-2-phenyl-3-(3,3,3-trifluoro-1-(thiophen-2-yl)propyl)-1H-indole
6-isothiocyanato-2-phenyl-3-(3,3,3-trifluoro-1-(thiophen-2-yl)propyl)-1H-indole
7-isothiocyanato-2-phenyl-3-(3,3,3-trifluoro-1-(thiophen-2-yl)propyl)-1H-indole
2-(2-isothiocyanatophenyl)-3-(3,3,3-trifluoro-1-(thiophen-2-yl)propyl)-1H-indole
2-(4-isothiocyanatophenyl)-3-(3,3,3-trifluoro-1-(thiophen-2-yl)propyl)-1H-indole
2-(3-isothiocyanatophenyl)-3-(3,3,3-trifluoro-1-(thiophen-2-yl)propyl)-1H-indole
5-(3,3,3-trifluoro-1-(2-phenyl-1H-indol-3-yl)propyl)thiophene-2-sulfonyl fluoride
5-(3,3,3-trifluoro-1-(2-phenyl-1H-indol-3-yl)propyl)thiophene-3-sulfonyl fluoride
2-(3,3,3-trifluoro-1-(2-phenyl-1H-indol-3-yl)propyl)thiophene-3-sulfonyl fluoride
2-phenyl-3-(3,3,3-trifluoro-1-(thiophen-2-yl)propyl)-1H-indole-4-sulfonyl fluoride
2-phenyl-3-(3,3,3-trifluoro-1-(thiophen-2-yl)propyl)-1H-indole-5-sulfonyl fluoride
2-phenyl-3-(3,3,3-trifluoro-1-(thiophen-2-yl)propyl)-1H-indole-6-sulfonyl fluoride
2-phenyl-3-(3,3,3-trifluoro-1-(thiophen-2-yl)propyl)-1H-indole-7-sulfonyl fluoride
2-(3-(3,3,3-trifluoro-1-(thiophen-2-yl)propyl)-1H-indol-2-yl)benzenesulfonyl fluoride
4-(3-(3,3,3-trifluoro-1-(thiophen-2-yl)propyl)-1H-indol-2-yl)benzenesulfonyl fluoride
3-(3-(3,3,3-trifluoro-1-(thiophen-2-yl)propyl)-1H-indol-2-yl)benzenesulfonyl fluoride
3-phenyl-3-(2-phenyl-1H-indol-3-yl) propyl nitrate
(2-(3-(nitrooxy)-1-(2-phenyl-1H-indol-3-yl)propyl)phenyl)boronic acid
(3-(3-(nitrooxy)-1-(2-phenyl-1H-indol-3-yl)propyl)phenyl)boronic acid
(4-(3-(nitrooxy)-1-(2-phenyl-1H-indol-3-yl)propyl)phenyl)boronic acid
(3-(3-(nitrooxy)-1-phenylpropyl)-2-phenyl-1H-indol-4-yl)boronic acid
(3-(3-(nitrooxy)-1-phenylpropyl)-2-phenyl-1H-indol-5-yl)boronic acid
(3-(3-(nitrooxy)-1-phenylpropyl)-2-phenyl-1H-indol-6-yl)boronic acid
(3-(3-(nitrooxy)-1-phenylpropyl)-2-phenyl-1H-indol-7-yl)boronic acid
(2-(3-(3-(nitrooxy)-1-phenylpropyl)-1H-indol-2-yl)phenyl)boronic acid
(3-(3-(3-(nitrooxy)-1-phenylpropyl)-1H-indol-2-yl)phenyl)boronic acid
(4-(3-(3-(nitrooxy)-1-phenylpropyl)-1H-indol-2-yl)phenyl)boronic acid
2-chloro-N-(2-(2-nitro-1-(2-phenyl-1H-indol-3-yl)ethyl)phenyl)acetamide
2-chloro-N-(3-(2-nitro-1-(2-phenyl-1H-indol-3-yl)ethyl)phenyl)acetamide
2-chloro-N-(4-(2-nitro-1-(2-phenyl-1H-indol-3-yl)ethyl)phenyl)acetamide
2-chloro-N-(3-(2-nitro-1-phenylethyl)-2-phenyl-1H-indol-5-yl)acetamide
2-chloro-N-(3-(2-nitro-1-phenylethyl)-2-phenyl-1H-indol-6-yl)acetamide
2-chloro-N-(2-(3-(2-nitro-1-phenylethyl)-1H-indol-2-yl)phenyl)acetamide
2-chloro-N-(3-(3-(2-nitro-1-phenylethyl)-1H-indol-2-yl)phenyl)acetamide
2-chloro-N-(4-(3-(2-nitro-1-phenylethyl)-1H-indol-2-yl)phenyl)acetamide
2-bromo-N-(2-(2-nitro-1-(2-phenyl-1H-indol-3-yl)ethyl)phenyl)acetamide
2-bromo-N-(3-(2-nitro-1-(2-phenyl-1H-indol-3-yl)ethyl)phenyl)acetamide
2-bromo-N-(4-(2-nitro-1-(2-phenyl-1H-indol-3-yl)ethyl)phenyl)acetamide
2-bromo-N-(3-(2-nitro-1-phenylethyl)-2-phenyl-1H-indol-5-yl)acetamide
2-bromo-N-(3-(2-nitro-1-phenylethyl)-2-phenyl-1H-indol-6-yl)acetamide
2-bromo-N-(2-(3-(2-nitro-1-phenylethyl)-1H-indol-2-yl)phenyl)acetamide
2-bromo-N-(3-(3-(2-nitro-1-phenylethyl)-1H-indol-2-yl)phenyl)acetamide
2-bromo-N-(4-(3-(2-nitro-1-phenylethyl)-1H-indol-2-yl)phenyl)acetamide
2-iodo-N-(2-(2-nitro-1-(2-phenyl-1H-indol-3-yl)ethyl)phenyl)acetamide
2-iodo-N-(3-(2-nitro-1-(2-phenyl-1H-indol-3-yl)ethyl)phenyl)acetamide
2-iodo-N-(4-(2-nitro-1-(2-phenyl-1H-indol-3-yl)ethyl)phenyl)acetamide
2-iodo-N-(3-(2-nitro-1-phenylethyl)-2-phenyl-1H-indol-5-yl)acetamide
2-iodo-N-(3-(2-nitro-1-phenylethyl)-2-phenyl-1H-indol-6-yl)acetamide
2-iodo-N-(2-(3-(2-nitro-1-phenylethyl)-1H-indol-2-yl)phenyl)acetamide
2-iodo-N-(3-(3-(2-nitro-1-phenylethyl)-1H-indol-2-yl)phenyl)acetamide
2-iodo-N-(4-(3-(2-nitro-1-phenylethyl)-1H-indol-2-yl)phenyl)acetamide
N-(2-(2-nitro-1-(2-phenyl-1H-indol-3-yl)ethyl)phenyl) acrylamide
N-(3-(2-nitro-1-(2-phenyl-1H-indol-3-yl)ethyl)phenyl) acrylamide
N-(4-(2-nitro-1-(2-phenyl-1H-indol-3-yl)ethyl)phenyl) acrylamide
N-(3-(2-nitro-1-phenylethyl)-2-phenyl-1H-indol-5-yl) acrylamide
N-(3-(2-nitro-1-phenylethyl)-2-phenyl-1H-indol-6-yl) acrylamide
N-(2-(3-(2-nitro-1-phenylethyl)-1H-indol-2-yl)phenyl) acrylamide
N-(3-(3-(2-nitro-1-phenylethyl)-1H-indol-2-yl)phenyl) acrylamide
N-(4-(3-(2-nitro-1-phenylethyl)-1H-indol-2-yl)phenyl) acrylamide
(2-(1-(2-(4-aminophenyl)-1H-indol-3-yl)-2-nitroethyl)phenyl)boronic acid
(3-(1-(2-(4-aminophenyl)-1H-indol-3-yl)-2-nitroethyl)phenyl)boronic acid
(3-(1-(2-(4-aminophenyl)-1H-indol-3-yl)-2-nitroethyl)phenyl)boronic acid
(4-(1-(6-cyano-2-phenyl-1H-indol-3-yl)-2-nitroethyl)phenyl)boronic acid
(3-(1-(6-cyano-2-phenyl-1H-indol-3-yl)-2-nitroethyl)phenyl)boronic acid
(5-(1-(6-cyano-2-phenyl-1H-indol-3-yl)-2-nitroethyl)thiophen-3-yl)boronic acid
(5-(1-(2-(4-aminophenyl)-1H-indol-3-yl)-2-nitroethyl)thiophen-3-yl)boronic acid
(4-(1-(2-(4-isothiocyanatophenyl)-1H-indol-3-yl)-2-nitroethyl)phenyl)boronic acid
(3-(1-(2-(4-isothiocyanatophenyl)-1H-indol-3-yl)-2-nitroethyl)phenyl)boronic acid
(5-(1-(2-(4-isothiocyanatophenyl)-1H-indol-3-yl)-2-nitroethyl)thiophen-3-yl)boronic acid
3-(2-nitro-1-(2-(4,4,5,5-tetramethyl-1,3,2-dioxaborolan-2-yl)phenyl)ethyl)-2-phenyl-1H-indole
3-(2-nitro-1-(3-(4,4,5,5-tetramethyl-1,3,2-dioxaborolan-2-yl)phenyl)ethyl)-2-phenyl-1H-indole
3-(2-nitro-1-(4-(4,4,5,5-tetramethyl-1,3,2-dioxaborolan-2-yl)phenyl)ethyl)-2-phenyl-1H-indole
3-(2-nitro-1-phenylethyl)-2-phenyl-5-(4,4,5,5-tetramethyl-1,3,2-dioxaborolan-2-yl)-1H-indole
3-(2-nitro-1-phenylethyl)-2-phenyl-6-(4,4,5,5-tetramethyl-1,3,2-dioxaborolan-2-yl)-1H-indole
3-(2-nitro-1-phenylethyl)-2-phenyl-7-(4,4,5,5-tetramethyl-1,3,2-dioxaborolan-2-yl)-1H-indole
3-(2-nitro-1-phenylethyl)-2-(4-(4,4,5,5-tetramethyl-1,3,2-dioxaborolan-2-yl)phenyl)-1H-indole
3-(2-nitro-1-phenylethyl)-2-(2-(4,4,5,5-tetramethyl-1,3,2-dioxaborolan-2-yl)phenyl)-1H-indole
3-(2-nitro-1-phenylethyl)-2-(3-(4,4,5,5-tetramethyl-1,3,2-dioxaborolan-2-yl)phenyl)-1H-indole
5-(3-(2-nitro-1-phenylethyl)-1H-indol-2-yl)benzo[c][1,2]oxaborol-1 (3H)-ol
5-(2-nitro-1-(2-phenyl-1H-indol-3-yl)ethyl)benzo[c][1,2]oxaborol-1 (3H)-ol
7-(2-nitro-1-phenylethyl)-6-phenyl-3,5-dihydro-1H-[1,2]oxaborolo [3,4-f]indol-1-ol.
(2-(2-nitro-1-(2-phenyl-1H-indol-3-yl)ethyl) furan-3-yl)boronic acid
(5-(2-nitro-1-(2-phenyl-1H-indol-3-yl)ethyl) furan-3-yl)boronic acid
(3-(3-(1-(furan-2-yl)-2-nitroethyl)-1H-indol-2-yl)phenyl)boronic acid
(4-(3-(1-(furan-2-yl)-2-nitroethyl)-1H-indol-2-yl)phenyl)boronic acid

In some embodiments, provided is a compound selected from those described above, or a pharmaceutically acceptable salt, or a stereoisomer thereof.

In certain embodiments, the present disclosure is directed to pro-drugs. Various forms of pro-drugs are known in the art, for example, as discussed in, for example, Bundgaard, (ed.), Design of Prodrugs, Elsevier (1985); Widder, et al., (ed.), Methods in Enzymology, vol. 4, Academic Press (1985); Krogsgaard Larsen, et al., (ed.), “Design and Application of Prodrugs”, Textbook of Drug Design and Development, Chapter 5, 113-191 (1991); Bundgaard, Journal of Drug Delivery Reviews, 8:1-38 (1992); Bundgaard, Journal of Pharmaceutical Sciences, 77:285 et seq. (1988); and Higuchi and Stella (eds.) Prodrugs as Novel Drug Delivery Systems, American Chemical Society (1975), each of which is incorporated by reference in its entirety.

It is understood that the claims encompass all possible stereoisomers and pro-drugs thereof, and their pharmaceutically acceptable salts thereof.

In certain embodiments, the compounds of the disclosure were synthesized in accordance with the schemes provided in the Examples below.

### Compositions

Provided herein is a composition comprising a compound disclosed herein (e.g., a compound of structural formula I, Ia, Ib, II, IIa, or IIb), or a pharmaceutically acceptable salt thereof, and a pharmaceutically acceptable carrier. In some embodiments, a composition of the present disclosure is formulated for oral, intravenous, subcutaneous, intraperitoneal, or dermatological administration to a subject in need of the composition.

The term “subject”, as used herein, means an animal, preferably a mammal, and most preferably a human.

The term “pharmaceutically acceptable carrier”, as used herein, shall encompass carriers, excipients, and diluents. Examples of carriers are well known to those skilled in the art and are prepared in accordance with acceptable pharmaceutical procedures, such as, for example, those described in Remington's Pharmaceutical Sciences, 17th edition, ed. Alfonso R. Gennaro, Mack Publishing Company, Easton, Pa. (1985), which is incorporated herein by reference in its entirety. Pharmaceutically acceptable carriers are those that are compatible with the other ingredients in the formulation and biologically acceptable.

The compounds or their pharmaceutically acceptable salts may be administered orally or parenterally, neat or in combination with conventional pharmaceutical carriers. Applicable solid carriers can include one or more substances which may also act as flavoring agents, lubricants, solubilizers, suspending agents, fillers, glidants, compression aids, binders, or tablet-disintegrating agents or encapsulating materials. Oral formulations may comprise any conventionally used oral forms, including tablets, capsules, buccal forms, troches, lozenges, and oral liquids, suspensions, or solutions. In powders, the carrier is a finely divided solid, which is an admixture with the finely divided active ingredient. In tablets, the active ingredient is mixed with a carrier having the necessary compression properties in suitable proportions and compacted in the shape and size desired. In one embodiment, the powders and capsules contain up to 99% of the active ingredient. Capsules may contain mixtures of the active compound(s) or their pharmaceutically acceptable salt(s) with inert fillers and/or diluents such as pharmaceutically acceptable starches (e.g., corn, potato, or tapioca starch), sugars, artificial sweetening agents, powdered celluloses, such as crystalline and microcrystalline celluloses, flours, gelatins, gums, etc. Useful tablet formulations may be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, and ion exchange resins.

In one embodiment, the surface modifying agent includes nonionic and anionic surface modifying agents. Representative examples of surface modifying agents include, but are not limited to, poloxamer 188, benzalkonium chloride, calcium stearate, cetostearyl alcohol, cetomacrogol emulsifying wax, sorbitan esters, colloidal silicon dioxide, phosphates, sodium dodecyl sulfate, magnesium aluminum silicate, and triethanolamine.

Oral formulations herein may utilize standard delay or time released formulations to alter the absorption of the active compound(s) or their pharmaceutically acceptable salt(s). The oral formulation may also consist of administering the active ingredient in water or fruit juice, containing appropriate solubilizers or emulsifiers as needed. Liquid carriers may be used in preparing solutions, suspensions, emulsions, syrups, and elixirs. The active ingredient of this disclosure may be dissolved or suspended in a pharmaceutically acceptable liquid carrier such as water, an organic solvent, a mixture of both or pharmaceutically acceptable oils or fat.

The liquid carrier can contain other suitable pharmaceutical additives such as solubilizers, emulsifiers, buffers, preservatives, sweeteners, flavorings agents, suspending agents, thickening agents, colors, viscosity regulators, stabilizers, or osmo-regulators. Suitable examples of liquid carriers for oral and parenteral administration include water (particularly containing additives as above, e.g., cellulose derivatives, sodium carboxymethylcellulose solution), alcohols (including monohydric and polyhydric alcohols, e.g., glycols) and their derivatives, and oils (e.g., fractionated coconut oil and arachis oil).

For parenteral administration the carrier can also be an oily ester such as ethyl oleate and/or isopropyl myristate. Sterile liquid carriers are used in sterile liquid form compositions for parenteral administration. The liquid carrier for pressurized compositions may be halogenated hydrocarbon or other pharmaceutically acceptable propellant. Liquid pharmaceutical compositions, which are sterile solutions or suspensions, may be utilized by, for example, intramuscular, intraperitoneal, or subcutaneous injection. Sterile solutions can also be administered intravenously.

Compositions for oral administration may be in either liquid or solid form. In one embodiment, the pharmaceutical composition is in unit dosage form, e.g., as tablets, capsules, powders, solutions, suspensions, emulsions, granules, or suppositories. In such form, the composition is sub-divided into unit dose(s) containing appropriate quantities of the active ingredient; the unit dosage forms may be packaged compositions, for example, packeted powders, vials, ampules, prefilled syringes, or sachets containing liquids.

The unit dosage form may be, for example, a capsule or tablet itself, or may be the appropriate number of any such compositions in package form. Such unit dosage form may contain from about 1 mg to about 1000 mg and may be given in a single dose or in two or more divided doses. Such doses may be administered in any manner useful in directing the active compound(s) herein to the recipient's bloodstream, including orally, via implants, parenterally (including intravenous, intraperitoneal, and subcutaneous injections), rectally and transdermally. Such administrations may be carried out using the compound(s) or their pharmaceutically acceptable salts disclosed herein in lotions, creams, foams, patches, suspensions, solutions, and suppositories (rectal). When administered for the treatment or inhibition of a particular disease state or disorder, it is understood that the effective dose may vary depending upon the particular compound or salt utilized, the mode of administration, the conditions, the severity of the condition being treated, as well as various physical factors related to the individual being treated.

The compositions of this disclosure may also be administered parenterally or intraperitoneally. Solutions or suspensions, of active compounds as a free base, a free acid, or pharmaceutically acceptable salt may be prepared in water suitably mixed with a surfactant such as hydroxypropyl cellulose. Dispersions may also be prepared in glycerol, liquid polyethylene glycols and mixtures thereof in oils. Under ordinary conditions of storage and use, these preparations contain a preservative to inhibit the growth of microorganisms. The pharmaceutical forms suitable for injectable use include sterile aqueous solutions or dispersions and sterile powders for extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi.

The carrier may be a solvent or dispersion medium containing, for example, water, ethanol, polyol (e.g., glycerol, propylene glycol, and liquid polyethylene glycol), suitable mixtures thereof, and vegetable oils.

### Methods

Also provided herein is a method for modulating (e.g., potentiating) the activity of endocannabinoids (e.g., 2-arachidonoylglycerol and/or anandamide) at the cannabinoid receptor type 1 (CB1), comprising contacting a cannabinoid type 1 receptor with an endocannabinoid (e.g., 2-arachidonoylglycerol and/or anandamide) and a compound (e.g., a compound of any of the structural formulas I, Ia, and Ib, II, IIa, IIb), salt or composition described herein.

Also provided herein is a method for modulating (e.g., inhibiting) the activity of enzymes of the endocannabinoid system, comprising contacting a compound (e.g., a compound of any of the structural formulas I, Ia, and Ib, II, IIa, IIb), salt or composition described herein.

Also provided herein is a method for treating a disease, disorder, or condition in which modulation of the endocannabinoid system is beneficial, in a subject in need thereof, comprising administering to the subject an effective amount of a compound (e.g., a compound of any of the structural formulas I, Ia, and Ib, II, IIa, IIb), salt of composition described herein. A “disease, disorder, or condition in which modulation of the endocannabinoid system is beneficial” refers to any disease or other deleterious disorder or condition in which the endocannabinoid system plays a role.

Examples of diseases, disorders, and conditions in which modulation of the endocannabinoid system is beneficial include, without limitations, neurological (e.g., multiple sclerosis, autism, Parkinson and Alzheimer disease, Tourette's syndrome, Huntington's disease, epilepsy, seizures) and eating (e.g., anorexia nervosa) disorders, neuropathic pain, other inflammatory types of pain, opioid dependence, cannabinoid overdose, opioid overdose, glaucoma, nausea and vomiting, post-traumatic stress disorder, and traumatic brain injury.

As used herein, the terms “treat”, “treating”, or “treatment” mean to counteract a medical condition, disorder, or disease to the extent that the medical condition, disorder, or disease is improved according to a clinically-acceptable standard.

In an embodiment, the subject (e.g., patient) is an animal, preferably a mammal (e.g., human, non-human primate, cow, sheep, goat, horse, dog, cat, rabbit, guinea pig, rat, mouse, or other bovine, ovine, equine, canine, feline, or rodent organism). In a particular embodiment, the subject is a human. The term “subject in need thereof” refers to a subject who has, or is at risk for developing a disease, disorder, or condition described herein (e.g., a disease, disorder, or condition in which modulation of the endocannabinoid system is beneficial). A skilled medical professional (e.g., physician) can readily determine whether a subject has, or is at risk for developing, a disease, disorder, or condition described herein.

The terms “effective amount”, “therapeutically effective amount”, and “effective dosage” as used herein, refer to the amount of a compound or salt that, when administered to a subject, is effective to ameliorate at least partially (and, in preferred embodiments, cure) a disease, disorder, or condition from which the subject is suspected to suffer. It is understood that the effective dosage may vary depending upon the particular compound or salt utilized, the mode of administration, the conditions, the severity of the condition being treated, as well as various physical factors related to the individual being treated.

An effective amount of the compound or salt to be administered can be determined by a clinician of ordinary skill using the guidance provided herein and other methods known in the art, and is dependent on several factors including, for example, the particular compound or salt chosen, the subject's age, sensitivity, tolerance to drugs, and overall well-being. For example, suitable dosages can be from about 0.001 mg/kg to about 1000 mg/kg, from about 0.01 mg/kg to about 100 mg/kg, from about 0.1 mg/kg to about 10 mg/kg body weight per treatment. Determining the dosage for a particular compound, salt, subject and diseases, condition, or disorder is well within the abilities of one skilled in the art. Preferably, the dosage does not cause or produces minimal adverse side effects (e.g., immunogenic response, nausea, dizziness, gastric upset, hyperviscosity syndrome, congestive heart failure, stroke, pulmonary edema).

A compound described herein (e.g., a compound of any structural formula I, Ia, Ib, II, IIa, IIb), or a pharmaceutically acceptable salt thereof, can be administered in a single dose or as multiple doses, for example, in an order and on a schedule suitable to achieve a desired therapeutic effect. Suitable dosages and regimens of administration can be determined by a clinician of ordinary skill.

A compound described herein (e.g., a compound of any structural formula I, Ia, Ib, II, IIa, IIb), or a pharmaceutically acceptable salt thereof, can be administered in combination with one or more other therapies or treatments. With respect to the administration of a compound or salt in combination with one or more other therapies or treatments, the compound or salt is typically administered as a single dose (by, e.g., infusion, injection, or orally), followed by repeated doses at particular intervals (e.g., one of more hours) if desired or indicated.

When administered in a combination therapy, the compound or salt can be administered before, concurrently, or after the other therapy (e.g., an additional agent(s)). When co-administered simultaneously (e.g., concurrently), the compound or salt and other therapy can be in separate formulations or in the same formulation. Alternatively, the compound or salt and other therapy can be administered sequentially, as separate compositions, within an appropriate time frame as determined by a skilled clinician (e.g., a time sufficient to allow an overlap of the therapeutic effect of the compositions).

A compound described herein (e.g., a compound of any structural formula I, Ia, Ib, II, Ila, IIb), or a pharmaceutically acceptable salt thereof, can be administered via a variety of routes of administration, including, for example, oral, dietary, topical, transdermal, rectal, parenteral (e.g., intra-arterial, intravenous, intramuscular, subcutaneous, and intradermal injections), intravenous infusion, and inhalation (e.g., intrabronchial, intranasal, or oral inhalation, intranasal drops) routes of administration, depending on the compound or salt and the particular disease, disorder, or condition to be treated. Administration can be local or systemic as indicated. The preferred mode of administration can vary depending on the particular compound or salt chosen salt and the particular disease, disorder, or condition to be treated.

The actual dose of a therapeutic agent and/or treatment regimen can be determined by the physician, taking into consideration the nature of the disease, other therapies being given, and the characteristics of the subject.

### EXEMPLIFICATION

As depicted in the Examples below, in certain exemplary embodiments, compounds were prepared followed general procedures. It will be appreciated that, although the general methods depict the synthesis of certain compounds of the present disclosure, the following general methods, and other methods known to one of ordinary skill in the art, can be applied to all compounds and subclasses and species of each of these compounds, as described herein.

The symbols and conventions used in the following description of processes, schemes, and examples, are consistent with those used in the contemporary scientific literature, for example, the Journal of the American Chemical Society or the Journal of Biological Chemistry.

| | |
|---|---|
| Boc | tert-butyloxycarbonyl |
| CH₃COONH₄ | ammonium acetate |
| CS₂ | carbon disulfide |
| CuI | copper(I) iodide |
| DIPEA | diisopropylethylamine |
| DMAP | N,N-dimethyl-4-aminopyridine |
| DMEDA | 1,2-dimethylethylenediamine |
| DMSO | dimethylsulfoxide |
| H₂O | water |
| HCl | hydrochloric acid |
| HPLC | high performance liquid chromatography |
| MgSO₄ | magnesium sulfate |
| MHz | megahertz |
| MS | mass spectrometry |
| MW | microwave |
| NaIO₄ | sodium periodate |
| NaN₃ | sodium azide |
| NMR | nuclear magnetic resonance |
| PPA | polyphosphoric acid |
| PdCl₂(dppf)CH₂Cl₂ | [1,1′-bis(diphenylphosphino)ferrocene]dichloropal- |
| | ladium(II), complex with dichloromethane |
| PCC | pyridinium chlorochromate |
| TLC | thin layer chromatography |
| THF | tetrahydrofuran |
| UV | ultraviolet |

### General Conditions and Analytical Methods

All solvents and chemical reagents used were commercially available and were used without further purification unless otherwise noted.

Flash column chromatography employed silica gel 60 (230-400 mesh) using a Biotage Isolera One. All compounds were demonstrated to be homogeneous by analytical TLC on pre-coated silica gel TLC plates (Merck, 60 F245 on glass, layer thickness 250 μm), and were visualized by UV light (254 nm).

The microwave reactions were conducted using a Biotage Initiator Microwave Synthesizer using standard protocols that are known in the art.

NMR spectra were recorded on a Bruker Ultra Shield 400 WB plus (¹H at 400 MHz, ¹³C at 101 MHz) or on a Varian INOVA-500 (¹H at 500 MHz) spectrometers. Deuterated solvents typically contained 0.03% to 0.05% v/v tetramethyl silane (TMS). Chemical shifts (δ) are reported in units of ppm relative to internal TMS. Multiplicities are indicated as br (broadened), s (singlet), d (doublet), t (triplet), q (quartet), p (quintet), m (multiplet) and coupling constants (J) are reported in hertz (Hz).

IR spectra were recorded on a Perkin Elmer Spectrum One FT-IR spectrometer. Peak strength is indicated as w (weak), s (strong) and br (broad).

Mass spectral data are reported in the form of m/z (intensity relative to base=100). Purities of compounds were determined by HPLC/MS analysis using a Waters MicroMass ZQ system [electrospray-ionization (ESI) with Waters-2525 binary gradient module coupled to a Photodiode Array Detector (Waters-2996) and ELS detector (Waters-2424) using a XTerra MS C18, 5 μm, 4.6 mm×50 mm column and acetonitrile/water and were >95%.

### General Method A

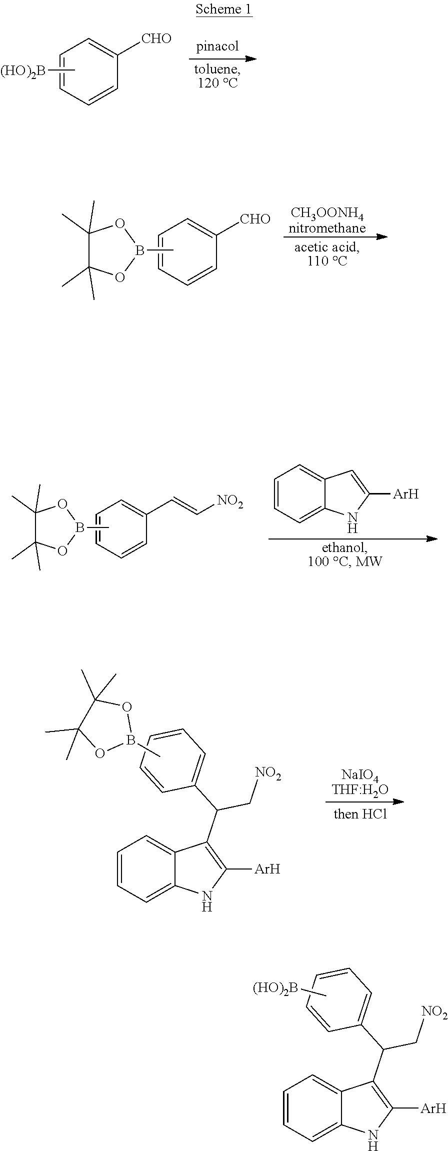

### Example 1. (4-(2-nitro-1-(2-phenyl-1H-indol-3-yl)ethyl)phenyl)boronic acid

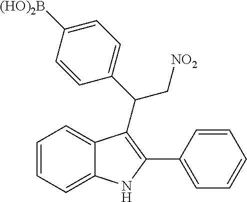 [SMILES: O=[N+]([O-])CC(c1ccc(B(O)O)cc1)c1c(-c2ccccc2)[nH]c2ccccc12]

### 4-(4,4,5,5-tetramethyl-1,3,2-dioxaborolan-2-yl)benzaldehyde

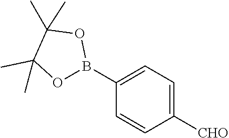 [SMILES: CC1(C)OB(c2ccc(C=O)cc2)OC1(C)C]

In a 250 mL round bottom flask, 4-formylbenzeneboronic acid (1 g, 6.6 mmol) and pinacol (857 mg, 7.3 mmol) were dissolved in anhydrous toluene (95 mL) under an argon atmosphere. The reaction mixture was refluxed for 14 hours at 120 °C. Toluene was evaporated under reduced pressure and the crude residue was purified with column chromatography (silica gel, hexanes/ethyl acetate 95:5) to give pure 4-(4,4,5,5-tetramethyl-1,3,2-dioxaborolan-2-yl)benzaldehyde as white solid (1.5 g, yield: 95%).

### (E)-4,4,5,5-tetramethyl-2-(4-(2-nitrovinyl)phenyl)-1,3,2-dioxaborolane

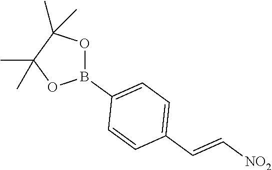 [SMILES: CC1(C)OB(c2ccc(/C=C/[N+](=O)[O-])cc2)OC1(C)C]

In a 50 mL round bottom flask 4-(4,4,5,5-tetramethyl-1,3,2-dioxaborolan-2-yl)benzaldehyde (1.2 g, 5.1 mmol), nitromethane (1.35 mL, 25.3 mmol) and ammonium acetate (1.56 g, 20.2 mmol) were dissolved in acetic acid (11 mL) and the reaction mixture was heated at 110 °C under an argon atmosphere for 4 hours. The reaction was then quenched with ice water and extracted with methylene chloride. The organic layer was dried over anhydrous MgSO₄, and the solvent was evaporated under reduced pressure. The residue was purified with column chromatography (silica gel, hexanes/diethyl ether 90:10) to give (E)-4,4,5,5-tetramethyl-2-(4-(2-nitrovinyl)phenyl)-1,3,2-dioxaborolane as yellow solid (1.1 g, yield: 78%).

### 3-(2-nitro-1-(4-(4,4,5,5-tetramethyl-1,3,2-dioxaborolan-2-yl)phenyl)ethyl)-2-phenyl-1H-indole

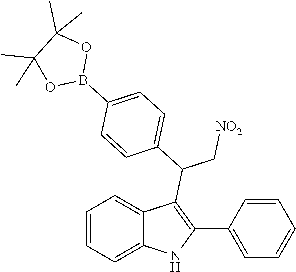 [SMILES: CC1(C)OB(c2ccc(C(C[N+](=O)[O-])c3c(-c4ccccc4)[nH]c4ccccc34)cc2)OC1(C)C]

In a 20 mL microwave tube (E)-4,4,5,5-tetramethyl-2-(4-(2-nitrovinyl)phenyl)-1,3,2-dioxaborolane (1 g, 3.6 mmol) and 2-phenylindole (700 mg, 3.6 mmol) were dispersed in anhydrous ethanol. The tube was sealed, and the reaction mixture was put under microwave irradiation at 100 °C for 45 minutes. After completion of the reaction, ethanol was evaporated under reduced pressure and the crude residue was purified with column chromatography (silica gel, hexanes/diethyl ether 80:20) to give 3-(2-nitro-1-(4-(4,4,5,5-tetramethyl-1,3,2-dioxaborolan-2-yl)phenyl)ethyl)-2-phenyl-1H-indole as pale-yellow solid (1.1 g, yield: 60%). ¹H NMR (500 MHz, Chloroform-d₃) δ 8.17 (s, 1H), 7.74 (d, J=8.1 Hz, 2H), 7.49 (d, J=9.1 Hz, 1H), 7.47-7.41 (m, 5H), 7.40 (dt, J=8.2, 0.9 Hz, 1H), 7.34 (d, J=7.7 Hz, 2H), 7.21 (t, J=7.1 Hz, 1H), 7.10 (t, J=7.1 Hz, 1H), 5.33 (t, J=7.9 Hz, 1H), 5.22-5.11 (m, 2H), 1.32 (s, 12H).

### (4-(2-nitro-1-(2-phenyl-1H-indol-3-yl)ethyl)phenyl)boronic acid

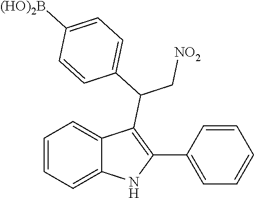 [SMILES: O=[N+]([O-])CC(c1ccc(B(O)O)cc1)c1c(-c2ccccc2)[nH]c2ccccc12]

In a 50 mL round bottom flask, 3-(2-nitro-1-(4-(4,4,5,5-tetramethyl-1,3,2-dioxaborolan-2-yl)phenyl)ethyl)-2-phenyl-1H-indole (600 mg, 1.3 mmol) and NaIO₄ (821 mg, 3.8 mmol) were dissolved in a mixture of THF:H₂O (10 ml: 2.8 ml) and left stirring at room temperature for 30 minutes. Then, aqueous hydrochloric acid solution 1M (0.9 mL, 0.896 mmol) was added and the reaction was stirred at room temperature for additional 17 hours. After completion, the reaction was quenched with water and extracted with methylene chloride. The organic layer was washed with brine, dried over anhydrous MgSO₄ and the solvent was evaporated under reduced pressure. The crude residue was purified with column chromatography (silica gel, hexanes/ethyl acetate 80:20 to 30:70) to give (4-(2-nitro-1-(2-phenyl-1H-indol-3-yl)ethyl)phenyl)boronic acid (Compound 1) as beige solid (310 mg, yield: 63%). ¹H NMR (500 MHz, DMSO-d₆) δ 11.44 (s, 1H), 7.69 (d, J=7.7 Hz, 2H), 7.61 (d, J=8.1 Hz, 1H), 7.58-7.45 (m, 5H), 7.39 (d, J=8.2 Hz, 1H), 7.27 (d, J=7.8 Hz, 2H), 7.12 (t, J=7.6 Hz, 1H), 6.98 (t, J=7.6 Hz, 1H), 5.55 (dd, J=13.1, 7.4 Hz, 1H), 5.42 (dd, J=13.1, 9.1 Hz, 1H), 5.20 (t, J=8.2 Hz, 1H).

### Example 2. (3-(2-nitro-1-(2-phenyl-1H-indol-3-yl)ethyl)phenyl)boronic acid

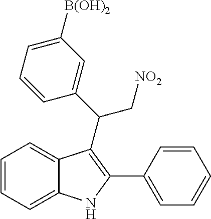 [SMILES: O=[N+]([O-])CC(c1cccc(B(O)O)c1)c1c(-c2ccccc2)[nH]c2ccccc12]

### 3-(4,4,5,5-tetramethyl-1,3,2-dioxaborolan-2-yl)benzaldehyde

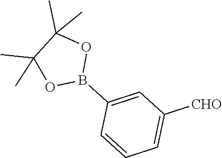 [SMILES: CC1(C)OB(c2cccc(C=O)c2)OC1(C)C]

In a 250 mL round bottom flask, 3-formylbenzeneboronic acid (1 g, 6.6 mmol) and pinacol (857 mg, 7.3 mmol) were dissolved in anhydrous toluene (95 mL) under an argon atmosphere. The reaction mixture was refluxed for 14 hours at 120 °C. Toluene was evaporated under reduced pressure and the crude residue was purified with column chromatography (silica gel, hexanes/ethyl acetate 95:5 to 90:10) to give pure 3-(4,4,5,5-tetramethyl-1,3,2-dioxaborolan-2-yl)benzaldehyde as white solid (1.45 g, yield: 93%).

### (E)-4,4,5,5-tetramethyl-2-(3-(2-nitrovinyl)phenyl)-1,3,2-dioxaborolane

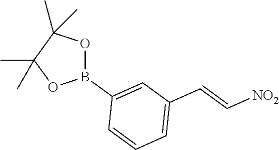 [SMILES: CC1(C)OB(c2cccc(/C=C/[N+](=O)[O-])c2)OC1(C)C]

In a 100 mL round bottom flask 3-(4,4,5,5-tetramethyl-1,3,2-dioxaborolan-2-yl)benzaldehyde (1.25 g, 5.3 mmol), nitromethane (1.4 mL, 26.4 mmol) and ammonium acetate (1.72 g, 21.1 mmol) were dissolved in acetic acid (11 mL) and the reaction mixture was heated at 110 °C under an argon atmosphere for 5 hours. The reaction was then quenched with ice water and extracted with methylene chloride. The organic layer was dried over anhydrous MgSO₄, and the solvent was evaporated under reduced pressure. The crude residue was purified with column chromatography (silica gel, hexanes/diethyl ether 90:10) to give (E)-4,4,5,5-tetramethyl-2-(3-(2-nitrovinyl)phenyl)-1,3,2-dioxaborolane as yellow solid (1.0 g, yield: 70%).

### 3-(2-nitro-1-(3-(4,4,5,5-tetramethyl-1,3,2-dioxaborolan-2-yl)phenyl)ethyl)-2-phenyl-1H-indole

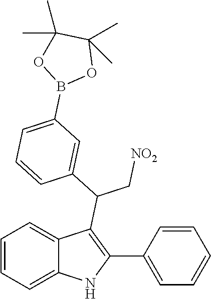 [SMILES: CC1(C)OB(c2cccc(C(C[N+](=O)[O-])c3c(-c4ccccc4)[nH]c4ccccc34)c2)OC1(C)C]

In a 20 mL microwave tube (E)-4,4,5,5-tetramethyl-2-(3-(2-nitrovinyl)phenyl)-1,3,2-dioxaborolane (143 mg, 0.52 mmol) and 2-phenylindole (100 mg, 0.52 mmol) were dispersed in anhydrous ethanol (2.6 mL). The tube was sealed, and the reaction mixture was put under microwave irradiation at 100 °C for 45 minutes. After completion of the reaction, ethanol was evaporated under reduced pressure and the crude residue was purified with column chromatography (silica gel, hexanes/ethyl acetate 80:20) to give 3-(2-nitro-1-(4-(4,4,5,5-tetramethyl-1,3,2-dioxaborolan-2-yl)phenyl)ethyl)-2-phenyl-1H-indole as pale-yellow solid (160 mg, yield: 66%). ¹H NMR (500 MHz, Chloroform-d) δ 8.18 (s, 1H), 7.80 (s, 1H), 7.68 (d, J=7.3 Hz, 1H), 7.51 (d, J=8.1 Hz, 1H), 7.46-7.36 (m, 7H), 7.29-7.24 (m, 1H), 7.21 (ddd, J=8.2, 7.0, 1.1 Hz, 1H), 7.10 (ddd, J=8.1, 7.0, 1.1 Hz, 1H), 5.34 (t, J=8.0 Hz, 1H), 5.18 (dd, J=8.0, 2.0 Hz, 2H), 1.34 (s, 12H).

### (3-(2-nitro-1-(2-phenyl-1H-indol-3-yl)ethyl)phenyl)boronic acid

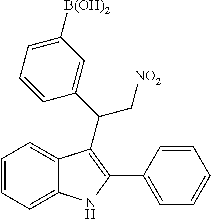 [SMILES: O=[N+]([O-])CC(c1cccc(B(O)O)c1)c1c(-c2ccccc2)[nH]c2ccccc12]

In a 25 mL round bottom flask, 3-(2-nitro-1-(3-(4,4,5,5-tetramethyl-1,3,2-dioxaborolan-2-yl)phenyl)ethyl)-2-phenyl-1H-indole (140 mg, 0.3 mmol) and NaIO₄ (192 mg, 0.9 mmol) were dissolved in a mixture of THF:H₂O (2.5 ml: 0.5 ml) and left stirring at room temperature for 30 minutes. Then, aqueous hydrochloric acid solution 1M (0.2 mL, 0.2 mmol) was added and the reaction was stirred at room temperature for additional 17 hours. After completion, the reaction was quenched with water and extracted with methylene chloride. The organic layer was washed with brine, dried over anhydrous MgSO₄ and the solvent was evaporated under reduced pressure. The crude residue was purified with column chromatography (silica gel, hexanes/ethyl acetate 80:20 to 40:60) to give (3-(2-nitro-1-(2-phenyl-1H-indol-3-yl)ethyl)phenyl)boronic acid (Compound 8) as orange solid (80 mg, yield: 68%), 1H NMR (500 MHz, DMSO-d₆) δ 11.44 (s, 1H), 7.79 (s, 1H), 7.61 (m, 2H), 7.55 (m, 4H), 7.51-7.45 (m, 1H), 7.40 (d, J=8.1 Hz, 1H), 7.33 (d, J=7.9 Hz, 1H), 7.27 (t, J=7.5 Hz, 1H), 7.12 (t, J=7.6 Hz, 1H), 6.99 (t, J=7.6 Hz, 1H), 5.50 (dd, J=13.0, 7.3 Hz, 1H), 5.41 (dd, J=13.1, 9.0 Hz, 1H), 5.21 (t, J=8.3 Hz, 1H).

### Example 3. (R)-(4-(2-nitro-1-(2-phenyl-1H-indol-3-yl)ethyl)phenyl)boronic acid

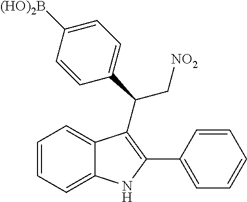 [SMILES: O=[N+]([O-])C[C@H](c1ccc(B(O)O)cc1)c1c(-c2ccccc2)[nH]c2ccccc12]

Chiral separation of (4-(2-nitro-1-(2-phenyl-1H-indol-3-yl)ethyl)phenyl)boronic acid was performed using the Agilent 1260 Infinity II HPLC system equipped with a CHIRALPAK-IC(5 μm, 10 mm×250 mm). Accordingly, (R)-(4-(2-nitro-1-(2-phenyl-1H-indol-3-yl)ethyl)phenyl)boronic acid (Compound 2; retention time 6.4 min) was obtained as a single enantiomer using 10% isopropanol in hexanes as eluent.

### Example 4. (S)-(4-(2-nitro-1-(2-phenyl-1H-indol-3-yl)ethyl)phenyl)boronic acid

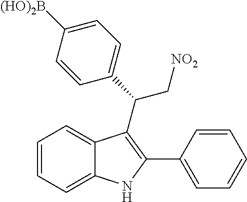 [SMILES: O=[N+]([O-])C[C@@H](c1ccc(B(O)O)cc1)c1c(-c2ccccc2)[nH]c2ccccc12]

Chiral separation of (4-(2-nitro-1-(2-phenyl-1H-indol-3-yl)ethyl)phenyl)boronic acid was performed using the Agilent 1260 Infinity HPLC system equipped with a CHIRALPAK-IC(5 μm, 10 mm×250 mm). Accordingly, (S)-(4-(2-nitro-1-(2-phenyl-1H-indol-3-yl)ethyl)phenyl)boronic acid (Compound 3; retention time 10.6 min) was obtained as a single enantiomer using 10% isopropanol in hexanes as eluent.

### General Method B

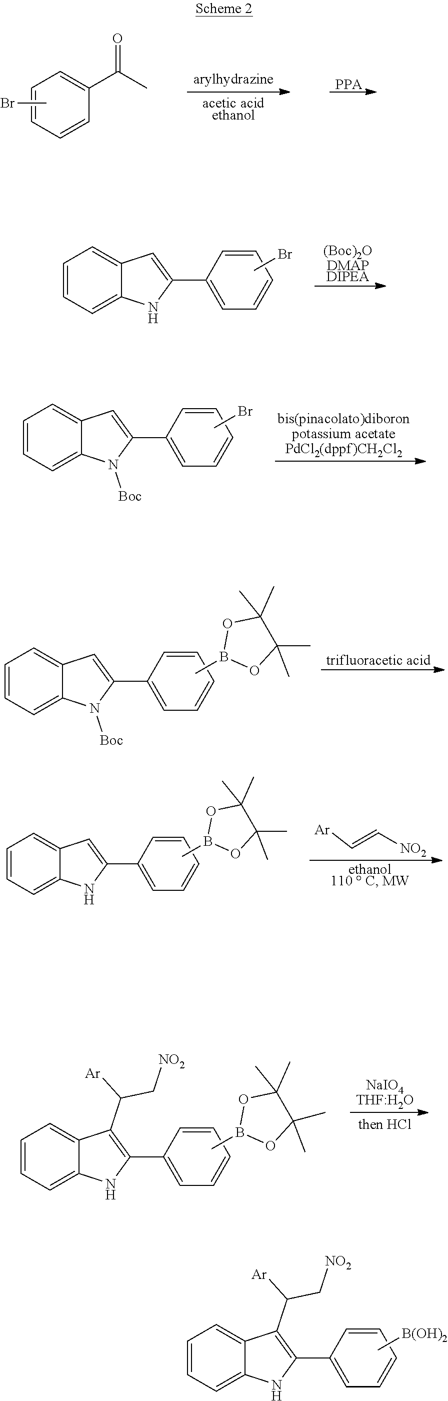

### Example 5. (4-(3-(2-nitro-1-phenylethyl)-1H-indol-2-yl)phenyl)boronic acid

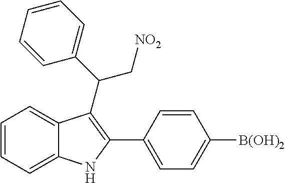 [SMILES: O=[N+]([O-])CC(c1ccccc1)c1c(-c2ccc(B(O)O)cc2)[nH]c2ccccc12]

### 2-(4-bromophenyl)-1H-indole

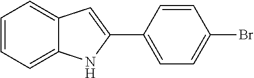 [SMILES: Brc1ccc(-c2cc3ccccc3[nH]2)cc1]

In a 100 mL round bottom flask, polyphosphoric acid was added and heated at 120 °C for 15 minutes. Then, 1-(4-bromophenyl) ethan-1-one (1.5 g, 7.5 mmol) and phenylhydrazine (0.9 ml, 9 mmol) were added and the reaction was heated at 120 °C for 90 minutes. The reaction was poured into ice-cold water and extracted with ethyl acetate. The organic layer was dried over MgSO₄, and the solvent was evaporated under reduced pressure. The crude residue was purified with column chromatography (silica gel, hexanes/ethyl acetate 95:5 to 80:20) to give 2-(4-bromophenyl)-1H-indole as white solid (1.7 g, yield: 81%).

### tert-butyl 2-(4-bromophenyl)-1H-indole-1-carboxylate

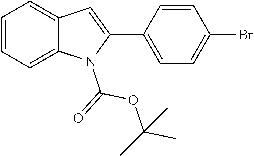 [SMILES: CC(C)(C)OC(=O)n1c(-c2ccc(Br)cc2)cc2ccccc21]

In a 50 mL round bottom flask 2-(4-bromophenyl)-1H-indole (300 mg, 1.1 mmol) was dissolved in anhydrous tetrahydrofuran (5.5 mL). Then, (Boc) 20 (324 mg, 1.48 mmol), DMAP (11 mg, 0.09 mmol) and DIPEA (0.33 mL, 1.9 mmol) were added, and the reaction was stirred at room temperature under an argon atmosphere for 19 hours. The reaction was quenched with water and extracted with ethyl acetate. The organic layer was dried over MgSO₄ and evaporated under reduced pressure. The crude residue was purified with column chromatography (silica gel, hexanes/ethyl acetate 95:5) to give tert-butyl 2-(4-bromophenyl)-1H-indole-1-carboxylate as colorless oil (380 mg, yield: 92%).

### tert-butyl 2-(4-(4,4,5,5-tetramethyl-1,3,2-dioxaborolan-2-yl)phenyl)-1H-indole-1-carboxylate

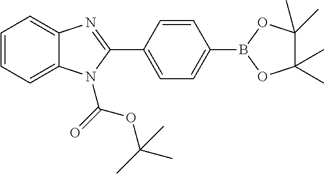 [SMILES: CC(C)(C)OC(=O)n1c(-c2ccc(B3OC(C)(C)C(C)(C)O3)cc2)nc2ccccc21]

In a 50 mL round bottom flask tert-butyl 2-(4-bromophenyl)-1H-indole-1-carboxylate (280 mg, 0.75 mmol) was dissolved in 1,4-dioxane (7.5 mL) and then bis(pinacolato)diboron (284 mg, 1.13 mmol) and potassium acetate (147 mg, 1.5 mmol) were added. The reaction mixture was bubbled with argon for 15 minutes before the addition of PdCl₂(dppf)CH₂Cl₂ (61 mg, 0.075 mmol). The reaction was heated at 80 °C under an argon atmosphere for 3 hours. The reaction was quenched with water and extracted with ethyl acetate. The organic phase was dried over MgSO₄ and evaporated under reduced pressure. The crude reside was purified with column chromatography (silica gel, hexanes/diethyl ether 80:20) to give tert-butyl 2-(4-(4,4,5,5-tetramethyl-1,3,2-dioxaborolan-2-yl)phenyl)-1H-indole-1-carboxylate as white solid (252 mg, yield: 80%).

### 2-(4-(4,4,5,5-tetramethyl-1,3,2-dioxaborolan-2-yl)phenyl)-1H-indole

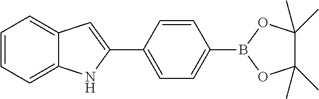 [SMILES: CC1(C)OB(c2ccc(-c3cc4ccccc4[nH]3)cc2)OC1(C)C]

In a 25 mL round bottom flask tert-butyl 2-(4-bromophenyl)-1H-indole-1-carboxylate (180 mg, 0.43 mmol) was dissolved in anhydrous methylene chloride (4.5 mL) and then was cooled to 0 °C. Trifluoracetic acid (0.5 mL, 6.45 mmol) was added, and the reaction was stirred at 0 °C for 10 minutes and then and left stirring overnight at room temperature. The reaction was quenched with sodium bicarbonate and extracted with ethyl acetate The organic layers were combined, dried over MgSO₄ and the solvent was evaporated under reduced pressure. The crude residue was purified with column chromatography (silica gel, hexanes/diethyl ether 60:40) to give 2-(4-(4,4,5,5-tetramethyl-1,3,2-dioxaborolan-2-yl)phenyl)-1H-indole as white solid (80 mg, yield 58%).

### 3-(2-nitro-1-phenylethyl)-2-(4-(4,4,5,5-tetramethyl-1,3,2-dioxaborolan-2-yl)phenyl)-1H-indole

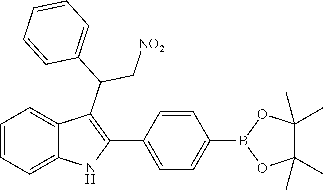 [SMILES: CC1(C)OB(c2ccc(-c3[nH]c4ccccc4c3C(C[N+](=O)[O-])c3ccccc3)cc2)OC1(C)C]

In a 20 mL microwave tube 2-(4-(4,4,5,5-tetramethyl-1,3,2-dioxaborolan-2-yl)phenyl)-1H-indole (50 mg, 0.16 mmol) and (E)-(2-nitrovinyl)benzene (30 mg, 0.19 mmol) were dispersed in anhydrous ethanol. The tube was sealed, and the reaction mixture was put under microwave irradiation at 100 °C for 45 minutes. After completion of the reaction, ethanol was evaporated under reduced pressure and the crude residue was purified with column chromatography (silica gel, hexanes/diethyl ether 80:20) to give 3-(2-nitro-1-phenylethyl)-2-(4-(4,4,5,5-tetramethyl-1,3,2-dioxaborolan-2-yl)phenyl)-1H-indole as yellow solid (49 mg, yield: 68%), 1H NMR (500 MHz, Chloroform-d) δ 8.23 (s, 1H), 7.93-7.88 (m, 2H), 7.55 (d, J=8.1 Hz, 1H), 7.48-7.43 (m, 2H), 7.40 (d, J=8.1 Hz, 1H), 7.34 (d, J=7.0 Hz, 2H), 7.29 (t, J=7.6 Hz, 2H), 7.25-7.20 (m, 2H), 7.12 (t, J=7.6 Hz, 1H), 5.32 (t, J=7.9 Hz, 1H), 5.19 (dd, J=12.5, 7.4 Hz, 1H), 5.12 (dd, J=12.6, 8.4 Hz, 1H), 1.37 (s, 12H).

### (4-(3-(2-nitro-1-phenylethyl)-1H-indol-2-yl)phenyl)boronic acid

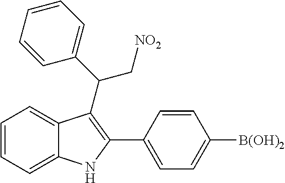 [SMILES: O=[N+]([O-])CC(c1ccccc1)c1c(-c2ccc(B(O)O)cc2)[nH]c2ccccc12]

In a 25 mL round-bottom flask, 3-(2-nitro-1-phenylethyl)-2-(4-(4,4,5,5-tetramethyl-1,3,2-dioxaborolan-2-yl)phenyl)-1H-indole (40 mg, 0.09 mmol) and NaIO₄ (55 mg, 0.26 mmol) were dissolved in a mixture of THF:H₂O (1 mL: 0.3 mL) and left stirring at room temperature for 30 minutes. Then, aqueous HCl 1M (60 μL, 0.06 mmol) was added and the reaction was left stirring at room temperature for 17 hours. After completion of the reaction, it was quenched with water and extracted with methylene chloride. The organic layers were combined, washed with brine, dried over MgSO₄ and the solvent was evaporated under reduced pressure. The crude residue was purified with column chromatography (silica gel, hexanes:ethyl acetate 80:20 and 30:70) to give (4-(3-(2-nitro-1-phenylethyl)-1H-indol-2-yl)phenyl)boronic acid (Compound 10) as white solid (23 mg, yield 72%). MS: m/z=387.1 [M+H]⁺

### Example 6. (4-(1-(2-(4-isothiocyanatophenyl)-1H-indol-3-yl)-2-nitroethyl)phenyl)boronic acid

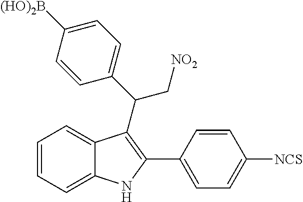

### 2-(4-azidophenyl)-1H-indole

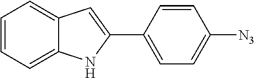 [SMILES: [N-]=[N+]=Nc1ccc(-c2cc3ccccc3[nH]2)cc1]

In a 50 mL round bottom flask 2-(4-bromophenyl)-1H-indole (600 mg, 2.2 mmol) was dissolved in a mixture of DMSO/H₂O (6 mL, 5:1) and NaN₃ (357 mg, 5.5 mmol) and sodium ascorbate (43 mg, 0.22 mmol) were added. The reaction was bubbled with argon for 5 minutes and then CuI (42 mg, 0.22 mmol) and DMEDA (0.07 mL, 0.66 mmol) were added. The reaction was heated to 80 °C for 6 hours. The reaction was quenched with water and extracted with methylene chloride. The organic layer was washed with brine and dried over MgSO₄. The solvent was evaporated under reduced pressure. The crude residue was purified with column chromatography (silica gel, hexanes/ethyl acetate 85:15) to give 2-(4-azidophenyl)-1H-indole as white solid (450 mg, yield: 87%).

### 2-(4-azidophenyl)-3-(2-nitro-1-(4-(4,4,5,5-tetramethyl-1,3,2-dioxaborolan-2-yl)phenyl)ethyl)-1H-indole

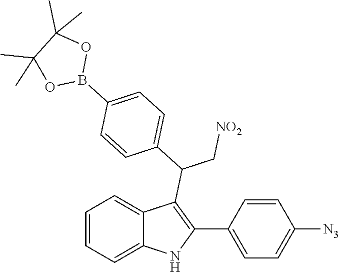 [SMILES: CC1(C)OB(c2ccc(C(C[N+](=O)[O-])c3c(-c4ccc(N=[N+]=[N-])cc4)[nH]c4ccccc34)cc2)OC1(C)C]

In a 20 mL microwave tube (E)-4,4,5,5-tetramethyl-2-(3-(2-nitrovinyl)phenyl)-1,3,2-dioxaborolane (500 g, 1.8 mmol) and 2-(4-azidophenyl)-1H-indole (420 mg, 1.8 mmol) were dispersed in anhydrous ethanol. The tube was sealed, and the reaction mixture was put under microwave irradiation at 100 °C for 45 minutes. After completion of the reaction, ethanol was evaporated under reduced pressure and the crude residue was purified with column chromatography (silica gel, hexanes/diethyl ether 85:15) to give 2-(4-azidophenyl)-3-(2-nitro-1-(4-(4,4,5,5-tetramethyl-1,3,2-dioxaborolan-2-yl)phenyl)ethyl)-1H-indole as yellow solid (570 mg, yield: 63%).

### (4-(1-(2-(4-azidophenyl)-1H-indol-3-yl)-2-nitroethyl)phenyl)boronic acid

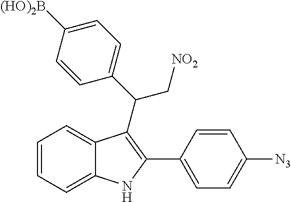 [SMILES: [N-]=[N+]=Nc1ccc(-c2[nH]c3ccccc3c2C(C[N+](=O)[O-])c2ccc(B(O)O)cc2)cc1]

In a 50 mL round bottom flask, 2-(4-azidophenyl)-3-(2-nitro-1-(4-(4,4,5,5-tetramethyl-1,3,2-dioxaborolan-2-yl)phenyl)ethyl)-1H-indole (509 mg, 1.0 mmol) and NaIO₄ (647 mg, 3.0 mmol) were dissolved in a mixture of THF:H₂O (7.8 ml: 2.2 ml) and left stirring at room temperature for 30 minutes. Then, aqueous hydrochloric acid solution 1M (0.7 mL, 0.7 mmol) was added and the reaction was stirred at room temperature for additional 17 hours. After completion, the reaction was quenched with water and extracted with methylene chloride. The organic layer was washed with brine, dried over anhydrous MgSO₄ and the solvent was evaporated under reduced pressure. The crude residue was purified with column chromatography (silica gel, hexanes/ethyl acetate 85:15 to 40:60) to give (4-(1-(2-(4-azidophenyl)-1H-indol-3-yl)-2-nitroethyl)phenyl)boronic acid as beige solid (278 mg, yield: 65%).

### (4-(1-(2-(4-isothiocyanatophenyl)-1H-indol-3-yl)-2-nitroethyl)phenyl)boronic acid

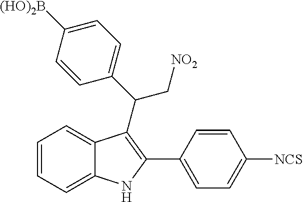

In a 25 mL round bottom flask (4-(1-(2-(4-azidophenyl)-1H-indol-3-yl)-2-nitroethyl)phenyl)boronic acid (90 mg, 0.21 mmol) was dissolved in anhydrous tetrahydrofuran (4 mL) and triphenylphosphine (275 mg, 1.05 mmol) and CS₂ (0.4 mL, 6.3 mmol) were added. The reaction was left stirring under an argon atmosphere at room temperature overnight. After completion, the reaction was quenched with water and extracted with ethyl acetate. The organic layer was washed with brine and dried over MgSO₄, and the solvents were evaporated under reduced pressure. The crude residue was purified with column chromatography (silica gel, ethyl acetate) to give (4-(1-(2-(4-isothiocyanatophenyl)-1H-indol-3-yl)-2-nitroethyl)phenyl)boronic acid (Compound 4) as brown solid (56 mg, yield 61%), 1H NMR (400 MHz, DMSO-d₆) δ 11.49 (s, 1H), 9.34 (s, 1H), 7.67 (m, 3H), 7.57 (d, J=8.3 Hz, 2H), 7.38 (d, J=8.2 Hz, 1H), 7.12 (dd, J=14.3, 7.7 Hz, 3H), 6.99 (t, J=7.5 Hz, 1H), 6.67 (d, J=8.3 Hz, 2H), 5.44 (dd, J=13.0, 9.3 Hz, 1H), 5.36 (dd, J=13.0, 9.3 Hz, 1H), 5.06 (t, J=8.3 Hz, 1H).

### Example 7. (4-(1-(2-(4-aminophenyl)-1H-indol-3-yl)-2-nitroethyl)phenyl) boronic acid

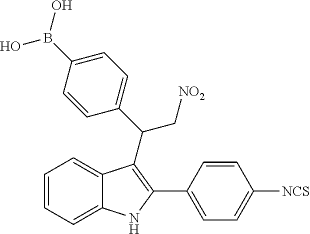

In a 25 mL round bottom flask (4-(1-(2-(4-azidophenyl)-1H-indol-3-yl)-2-nitroethyl)phenyl)boronic acid (120 mg, 0.28 mmol) was dissolved in tetrahydrofuran: H₂O (4 mL, 3:1) and triphenylphosphine (367 mg, 1.4 mmol) was added. The reaction was left stirring under an argon atmosphere at room temperature overnight. After completion, the reaction was quenched with water and extracted with ethyl acetate. The organic layer was washed with brine and dried over MgSO₄, and the solvents were evaporated under reduced pressure. The crude residue was purified with column chromatography (silica gel, ethyl acetate) to give (4-(1-(2-(4-aminophenyl)-1H-indol-3-yl)-2-nitroethyl)phenyl)boronic acid (Compound 9) as brown solid (45 mg, yield 40%), 1H NMR (400 MHz, DMSO-d₆) δ 11.12 (s, 1H), 7.95 (s, 1H), 7.67 (d, J=8.0 Hz, 1H), 7.53 (d, J=8.0 Hz, 1H), 7.28 (dd, J=17.2, 8.2 Hz, 3H), 7.19 (d, J=8.2 Hz, 2H), 7.03 (t, J=7.7 Hz, 1H), 6.91 (t, J=7.6 Hz, 1H), 6.68 (d, J=8.5 Hz, 1H), 5.51 (dd, J=13.1, 7.7 Hz, 1H), 5.40-5.30 (m, 3H), 5.17 (t, J=8.2 Hz, 1H).

### General Method C

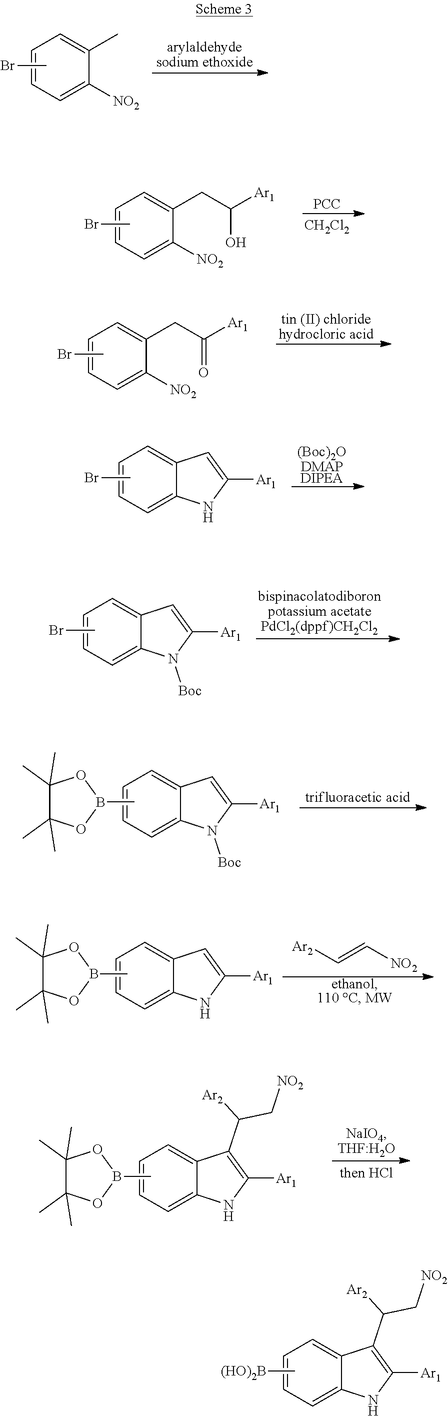

### Example 8. (3-(2-nitro-1-phenylethyl)-2-phenyl-1H-indol-6-yl)boronic acid

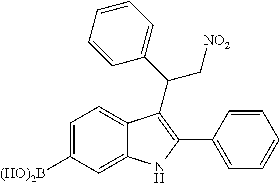 [SMILES: O=[N+]([O-])CC(c1ccccc1)c1c(-c2ccccc2)[nH]c2cc(B(O)O)ccc12]

### 2-(4-bromo-2-nitrophenyl)-1-phenylethan-1-ol

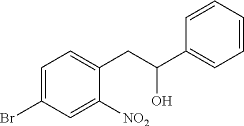 [SMILES: O=[N+]([O-])c1cc(Br)ccc1CC(O)c1ccccc1]

In a 100 mL round bottom flask 4-bromo-1-methyl-2-nitrobenzene (1.5 g, 6.9 mmol) and benzaldehyde (0.65 mL, 6.32 mmol) were dissolved in anhydrous DMSO (35 mL) and then sodium ethoxide in ethanol (1.84 mL from a solution 0.43 M) was added. The reaction was left stirring under an argon atmosphere for 14 hours. The reaction was quenched with water and extracted with ethyl acetate. The organic layer was washed with brine, dried over MgSO₄ and evaporated under reduced pressure. The crude residue was purified with column chromatography (silica gel, hexanes/ethyl acetate 90:10 to 70:30) to give 2-(4-bromo-2-nitrophenyl)-1-phenylethan-1-ol as white solid (1.17 g, yield 52%).

### 2-(4-bromo-2-nitrophenyl)-1-phenylethan-1-one

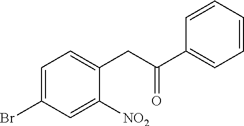 [SMILES: O=C(Cc1ccc(Br)cc1[N+](=O)[O-])c1ccccc1]

In a 100 mL round bottom flask 2-(4-bromo-2-nitrophenyl)-1-phenylethan-1-ol: 4-bromo-1-methyl-2-nitrobenzene (500 mg, 1.55 mmol) was dissolved in anhydrous methylene chloride (12 mL). Then, a suspension of PCC(534 mg, 2.48 mmol) in anhydrous methylene chloride (4 mL) was added and the reaction mixture was stirred at room temperature for 2 hours. The reaction was quenched with water and extracted with ethyl acetate. The organic phase was dried over MgSO₄ and evaporated under reduced pressure. The crude residue was purified with column chromatography (silica gel, hexanes/ethyl acetate 80:20) to give 2-(4-bromo-2-nitrophenyl)-1-phenylethan-1-one as orange solid (350 mg, yield: 70%).

### 6-bromo-2-phenyl-1H-indole

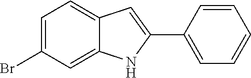 [SMILES: Brc1ccc2cc(-c3ccccc3)[nH]c2c1]

In a 100 mL round bottom flask 2-(4-bromo-2-nitrophenyl)-1-phenylethan-1-one (360 mg, 1.12 mmol), tin (II) chloride dihydrate (3.8 g, 16.9 mmol) and IN aqueous HCl (2.5 mL) in glacial acetic acid (14 mL) was heated at reflux for 13 hours. The reaction was quenched with saturated aqueous solution of potassium carbonate and extracted with ethyl acetate. The organic layer was washed with brine, dried over MgSO₄ and evaporated under reduced pressure. The crude residue was purified with column chromatography (silica gel, hexanes/ethyl acetate 90:10) to give 6-bromo-2-phenyl-1H-indole as white solid (67 mg, yield: 22%).

### tert-butyl 6-bromo-2-phenyl-1H-indole-1-carboxylate

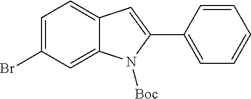 [SMILES: CC(C)(C)OC(=O)n1c(-c2ccccc2)cc2ccc(Br)cc21]

In a 25 mL round bottom flask 6-bromo-2-phenyl-1H-indole (100 mg, 0.34 mmol) was dissolved in anhydrous tetrahydrofuran (1.8 mL). Then, (Boc) 20 (103 mg, 0.51 mmol), DMAP (4 mg, 0.03 mmol) and DIPEA (0.11 mL, 0.63 mmol) were added, and the reaction was stirred at room temperature under an argon atmosphere for 19 hours. The reaction was quenched with water and extracted with ethyl acetate. The organic layer was dried over MgSO₄ and evaporated under reduced pressure. The crude residue was purified with column chromatography (silica gel, hexanes/ethyl acetate 95:5) to give tert-butyl 6-bromo-2-phenyl-1H-indole-1-carboxylate as colorless oil (129 mg, yield: 93%).

### tert-butyl 2-phenyl-6-(4,4,5,5-tetramethyl-1,3,2-dioxaborolan-2-yl)-1H-indole-1-carboxylate

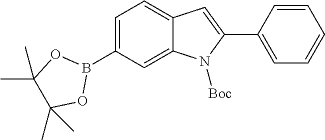 [SMILES: CC(C)(C)OC(=O)n1c(-c2ccccc2)cc2ccc(B3OC(C)(C)C(C)(C)O3)cc21]

In a 25 mL round bottom flask tert-butyl 6-bromo-2-phenyl-1H-indole-1-carboxylate (168 mg, 0.45 mmol) was dissolved in 1,4-dioxane (4.5 mL) and then bis(pinacolato)diboron (170 mg, 0.68 mmol) and potassium acetate (88 mg, 0.9 mmol) were added. The reaction mixture was bubbled with argon for 15 minutes before the addition of PdCl₂ (dppf) CH₂Cl₂ (37 mg, 0.045 mmol). The reaction was heated at 80 °C under an argon atmosphere for 3 hours. The reaction was quenched with water and extracted with ethyl acetate. The organic phase was dried over MgSO₄ and evaporated under reduced pressure. The crude reside was purified with column chromatography (silica gel, hexanes/diethyl ether 80:20) to give tert-butyl 2-phenyl-6-(4,4,5,5-tetramethyl-1,3,2-dioxaborolan-2-yl)-1H-indole-1-carboxylate as white solid (147 mg, yield: 78%).

### 2-phenyl-6-(4,4,5,5-tetramethyl-1,3,2-dioxaborolan-2-yl)-1H-indole

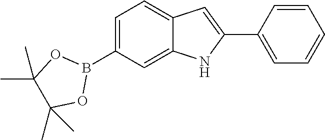 [SMILES: CC1(C)OB(c2ccc3cc(-c4ccccc4)[nH]c3c2)OC1(C)C]

In a 25 mL round bottom flask tert-butyl 2-phenyl-6-(4,4,5,5-tetramethyl-1,3,2-dioxaborolan-2-yl)-1H-indole-1-carboxylate (90 mg, 0.22 mmol) was dissolved in anhydrous methylene chloride (2.3 mL) and then was cooled to 0 °C. Trifluoracetic acid (0.25 mL, 3.2 mmol) was added, and the reaction was stirred at 0 °C for 10 minutes and then and left stirring overnight at room temperature. The reaction was quenched with sodium bicarbonate and extracted with ethyl acetate The organic layers were combined, dried over MgSO₄ and the solvent was evaporated under reduced pressure. The crude residue was purified with column chromatography (silica gel, hexanes/diethyl ether 60:40) to give 2-phenyl-6-(4,4,5,5-tetramethyl-1,3,2-dioxaborolan-2-yl)-1H-indole as white solid (40 mg, yield 57%).

### 3-(2-nitro-1-phenylethyl)-2-phenyl-6-(4,4,5,5-tetramethyl-1,3,2-dioxaborolan-2-yl)-1H-indole

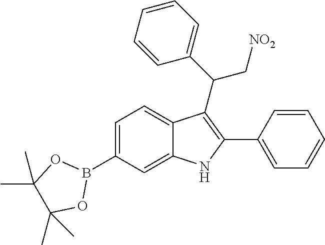 [SMILES: CC1(C)OB(c2ccc3c(C(C[N+](=O)[O-])c4ccccc4)c(-c4ccccc4)[nH]c3c2)OC1(C)C]

In a 20 mL microwave tube (E)-(2-nitrovinyl)benzene (300 mg, 1.9 mmol) and 2-phenyl-6-(4,4,5,5-tetramethyl-1,3,2-dioxaborolan-2-yl)-1H-indole (600 mg, 1.9 mmol) were dispersed in anhydrous ethanol. The tube was sealed, and the reaction mixture was put under microwave irradiation at 100 °C for 45 minutes. After completion of the reaction, ethanol was evaporated under reduced pressure and the crude residue was purified with column chromatography (silica gel, hexanes/diethyl ether 80:20) to give 3-(2-nitro-1-phenylethyl)-2-phenyl-6-(4,4,5,5-tetramethyl-1,3,2-dioxaborolan-2-yl)-1H-indole as pale-yellow solid (540 mg, yield: 62%). MS: m/z=469.2 [M+H]⁺

### (3-(2-nitro-1-phenylethyl)-2-phenyl-1H-indol-6-yl)boronic acid

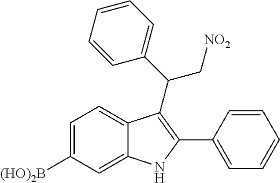 [SMILES: O=[N+]([O-])CC(c1ccccc1)c1c(-c2ccccc2)[nH]c2cc(B(O)O)ccc12]

In a 50 mL round bottom flask, 3-(2-nitro-1-phenylethyl)-2-phenyl-6-(4,4,5,5-tetramethyl-1,3,2-dioxaborolan-2-yl)-1H-indole (500 mg, 1.1 mmol) and NaIO₄ (690 mg, 3.2 mmol) were dissolved in a mixture of THF:H₂O (8.4 ml: 2.4 ml) and left stirring at room temperature for 30 minutes. Then, aqueous hydrochloric acid solution 1M (0.76 mL, 0.76 mmol) was added and the reaction was stirred at room temperature for additional 17 hours. After completion, the reaction was quenched with water and extracted with methylene chloride. The organic layer was washed with brine, dried over anhydrous MgSO₄ and the solvent was evaporated under reduced pressure. The crude residue was purified with column chromatography (silica gel, hexanes/ethyl acetate 80:20 to 30:70) to give (3-(2-nitro-1-phenylethyl)-2-phenyl-1H-indol-6-yl)boronic acid as yellow solid (260 mg, yield: 61%). MS: m/z=387.1 [M+H]⁺

### General Method D

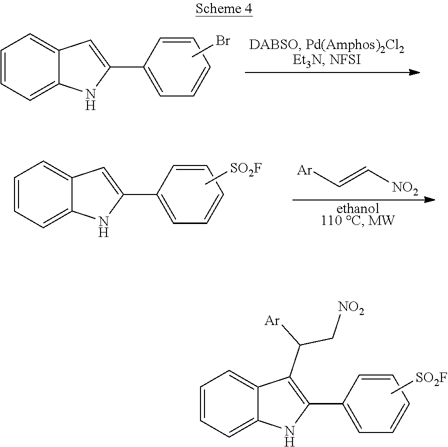

### Example 9. 4-(3-(2-nitro-1-phenylethyl)-1H-indol-2-yl)benzenesulfonyl fluoride

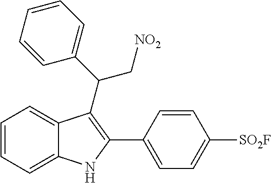 [SMILES: O=[N+]([O-])CC(c1ccccc1)c1c(-c2ccc(S(=O)(=O)F)cc2)[nH]c2ccccc12]

### 4-(1H-indol-2-yl)benzenesulfonyl fluoride

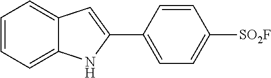 [SMILES: O=S(=O)(F)c1ccc(-c2cc3ccccc3[nH]2)cc1]

In a 50 mL round bottom flask 2-(4-bromophenyl)-1H-indole (120 mg, 0.44 mmol) was dissolved in iPrOH (2.2 mL) and DABSO (105 mg, 0.44 mmol), Pd(Amphos)₂Cl₂ (15 mg, 0.022 mmol) and Et₃N (0.2 mL, 1.32 mmol) were added. The reaction was heated at 75 °C for 24 hrs. The reaction mixture was cooled to room temperature, NFSI (208 mg, 0.66 mmol) was added and the reaction was stirred for 3 hours. Solvent was removed under reduced pressure, and the mixture was diluted with EtOAc and then filtered through a Celite pad. The filtrate was washed with saturated aqueous Na₂S₂O₃ and brine, dried over MgSO₄, and concentrated under reduced pressure. The crude residue was purified with column chromatography (silica gel, hexanes/ethyl acetate 90:10 to 30:70) to give 4-(1H-indol-2-yl)benzenesulfonyl fluoride as a pale yellow solid (90 mg, yield: 74%). ¹H NMR (500 MHz, CDCl₃) δ 8.43 (s, 1H), 8.07 (d, J=8.5 Hz, 2H), 7.88 (d, J=8.3 Hz, 2H), 7.68 (d, J=8.0 Hz, 1H), 7.45 (d, J=8.3 Hz, 1H), 7.29 (t, 1H), 7.17 (dd, J=8.1, 7.0 Hz, 1H), 7.05 (s, 1H).

### 4-(3-(2-nitro-1-phenylethyl)-1H-indol-2-yl)benzenesulfonyl fluoride

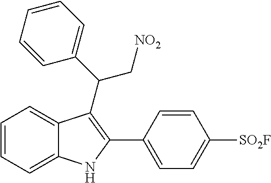 [SMILES: O=[N+]([O-])CC(c1ccccc1)c1c(-c2ccc(S(=O)(=O)F)cc2)[nH]c2ccccc12]

In a 5 mL microwave tube (E)-(2-nitrovinyl)benzene (50 mg, 0.216 mmol) and 4-(1H-indol-2-yl)benzenesulfonyl fluoride (50 mg, 0.18 mmol) were dispersed in anhydrous ethanol (2 mL). The tube was sealed, and the reaction mixture was put under microwave irradiation at 100 °C for 45 minutes. After completion of the reaction, ethanol was evaporated under reduced pressure and the crude residue was purified with column chromatography (silica gel, hexanes/diethyl ether 80:20) to give 4-(3-(2-nitro-1-phenylethyl)-1H-indol-2-yl)benzenesulfonyl fluoride (Compound 5) as a yellow solid (62 mg, yield: 80%), ¹H NMR (500 MHz, CDCl₃) δ 8.75 (s, 1H), 8.10 (d, J=8.2 Hz, 2H), 7.72 (d, J=8.2 Hz, 2H), 7.60 (d, J=8.1 Hz, 1H), 7.48 (d, J=8.2 Hz, 1H), 7.33 (m, 6H), 7.19 (t, J=7.6 Hz, 1H), 5.32-5.21 (m, 2H), 5.18 (dd, J=12.0, 6.2, 1H). MS: m/z=425.7 [M+H]⁺

### General Method E

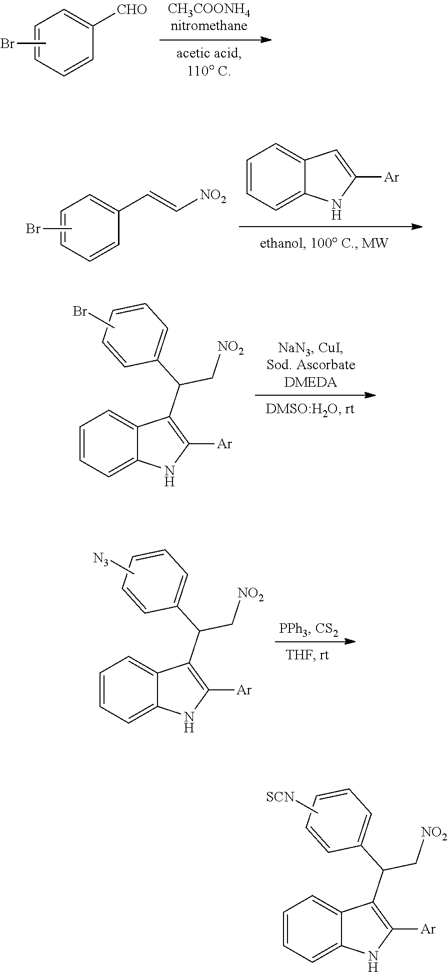

### Example 10. 3-(1-(4-isothiocyanatophenyl)-2-nitroethyl)-2-phenyl-1H-indole

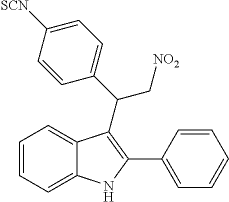

### (E)-1-iodo-4-(2-nitrovinyl)benzene

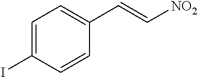 [SMILES: O=[N+]([O-])/C=C/c1ccc(I)cc1]

In a 250 mL round bottom flask 4-iodobenzaldehyde (2.6 g, 11.2 mmol), nitromethane (1.8 mL, 33.6 mmol) and ammonium acetate (2.16 g, 28.0 mmol) were dissolved in acetic acid (23 mL) and the reaction mixture was heated at 110 °C under an argon atmosphere for 5 hours. The reaction was then quenched with ice water and extracted with ethyl acetate. The organic layer was dried over anhydrous MgSO₄, and the solvent was evaporated under reduced pressure. The crude residue was purified with column chromatography (silica gel, hexanes/ethyl acetate 90:10) to give (E)-1-iodo-4-(2-nitrovinyl)benzene as a yellow solid (2.31 g, yield: 75%).

### 3-(1-(4-iodophenyl)-2-nitroethyl)-2-phenyl-1H-indole

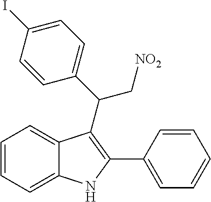 [SMILES: O=[N+]([O-])CC(c1ccc(I)cc1)c1c(-c2ccccc2)[nH]c2ccccc12]

In a 20 mL microwave tube (E)-1-iodo-4-(2-nitrovinyl)benzene (900 mg, 3.27 mmol) and 2-phenyl-1H-indole (631 mg, 3.27 mmol) were dispersed in anhydrous ethanol. The tube was sealed, and the reaction mixture was put under microwave irradiation at 100 °C for 45 minutes. After completion of the reaction, ethanol was evaporated under reduced pressure and the crude residue was purified with column chromatography (silica gel, hexanes/diethyl acetate 80:20) to give 3-(1-(4-iodophenyl)-2-nitroethyl)-2-phenyl-1H-indole as a yellow solid (920 mg, yield: 60%), ¹H NMR (500 MHz, CDCl₃) δ 8.18 (s, 1H), 7.61 (m, 2H), 7.50-7.37 (m, 7H), 7.23 (dd, J=8.2, 7.0 Hz, 1H), 7.13 (dd, J=8.1, 7.0 Hz, 1H), 7.06 (m, 2H), 5.25 (t, J=7.9 Hz, 1H), 5.15 (dd, J=12.6, 7.5, 1H), 5.09 (dd, J=12.5, 8.3 Hz, 1H).

### 3-(1-(4-azidophenyl)-2-nitroethyl)-2-phenyl-1H-indole

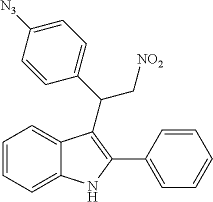 [SMILES: [N-]=[N+]=Nc1ccc(C(C[N+](=O)[O-])c2c(-c3ccccc3)[nH]c3ccccc23)cc1]

In a 100 mL round bottom flask 3-(1-(4-iodophenyl)-2-nitroethyl)-2-phenyl-1H-indole (1.5 g, 3.2 mmol) was dissolved in a mixture of DMSO/H₂O (16 mL, 5:1) and NaN₃ (416 mg, 6.4 mmol) and sodium ascorbate (63 mg, 0.32 mmol) were added. The reaction was bubbled with argon for 5 minutes and then CuI (60 mg, 0.32 mmol) and DMEDA (0.1 mL, 0.96 mmol) were added. The reaction was heated to 80 °C for 6 hours. The reaction was quenched with water and extracted with ethyl acetate. The organic layer was washed with brine and dried over MgSO₄. The solvent was evaporated under reduced pressure. The crude residue was purified with column chromatography (silica gel, hexanes/ethyl acetate 80:20) to give 3-(1-(4-azidophenyl)-2-nitroethyl)-2-phenyl-1H-indole as white solid (940 mg, yield: 76%), 1H NMR (500 MHz, (CD₃)CO) δ 10.57 (s, 1H), 7.67 (d, J=8 Hz, 1H), 7.58 (m, 2H), 7.53 (m, 2H), 7.46 (m, 4H), 7.16 (t, J=7.6 Hz, 1H), 7.05 (m, 3H), 5.49 (dd, J=12.5, 7.3 Hz, 1H), 5.41 (t, J=7.3 Hz, 1H), 5.37 (dd, J=12.5, 7.9 Hz, 1H).

### 3-(1-(4-isothiocyanatophenyl)-2-nitroethyl)-2-phenyl-1H-indole

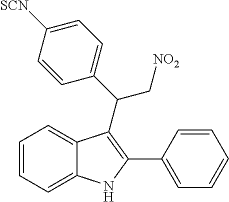

In a 250 mL round bottom flask 3-(1-(4-azidophenyl)-2-nitroethyl)-2-phenyl-1H-indole (470 mg, 1.23 mmol) was dissolved in anhydrous tetrahydrofuran (24 mL) and triphenylphosphine (1.6 mg, 6.15 mmol) and CS₂ (2.2 mL, 36.9 mmol) were added. The reaction was left stirring under an argon atmosphere at room temperature overnight. After completion, the reaction was quenched with water and extracted with ethyl acetate. The organic layer was washed with brine and dried over MgSO₄, and the solvents were evaporated under reduced pressure. The crude residue was purified with column chromatography (silica gel, hexane/ethyl acetate 80:20) to give 3-(1-(4-isothiocyanatophenyl)-2-nitroethyl)-2-phenyl-1H-indole (Compound 7) as a white solid (318 mg, yield 65%), 1H NMR (400 MHz, CDCl₃) δ 8.19 (s, 1H), 7.47 (m, 4H), 7.43 (m, 3H), 7.33 (d, J=8.2 Hz, 2H), 7.26 (t, J=8.3 Hz, 1H), 7.15 (m, 3H), 6.91 (t, J=7.6 Hz, 1H), 5.32 (t, J=7.9 Hz, 1H), 5.20 (dd, J=12.7, 7.7, 1H), 5.11 (dd, J=12.5, 7.9 Hz, 1H).

### Example 11. 2-(4-isothiocyanatophenyl)-3-(1-(4-isothiocyanatophenyl)-2-nitroethyl)-1H-indole

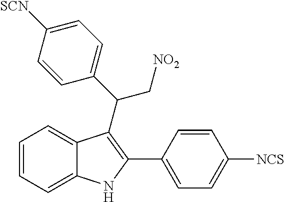

### 2-(4-iodophenyl)-3-(1-(4-iodophenyl)-2-nitroethyl)-1H-indole

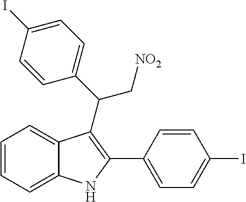 [SMILES: O=[N+]([O-])CC(c1ccc(I)cc1)c1c(-c2ccc(I)cc2)[nH]c2ccccc12]

In a 20 mL microwave tube (E)-1-iodo-4-(2-nitrovinyl)benzene (850 mg, 3.09 mmol) and 2-(4-iodophenyl)-1H-indole (985 mg, 3.09 mmol) were dispersed in anhydrous ethanol. The tube was sealed, and the reaction mixture was put under microwave irradiation at 100 °C for 45 minutes. After completion of the reaction, ethanol was evaporated under reduced pressure and the crude residue was purified with column chromatography (silica gel, hexanes/diethyl acetate 90:10) to give 2-(4-iodophenyl)-3-(1-(4-iodophenyl)-2-nitroethyl)-1H-indole as a yellow solid (980 mg, yield: 62%).

### 2-(4-azidophenyl)-3-(1-(4-azidophenyl)-2-nitroethyl)-1H-indole

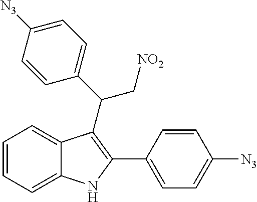 [SMILES: [N-]=[N+]=Nc1ccc(-c2[nH]c3ccccc3c2C(C[N+](=O)[O-])c2ccc(N=[N+]=[N-])cc2)cc1]

In a 100 mL round bottom flask 2-(4-iodophenyl)-3-(1-(4-iodophenyl)-2-nitroethyl)-1H-indole (900 mg, 1.5 mmol) was dissolved in a mixture of DMSO/H₂O (12 mL, 5:1) and NaN₃ (416 mg, 6.4 mmol) and sodium ascorbate (63 mg, 0.32 mmol) were added. The reaction was bubbled with argon for 5 minutes and then CuI (60 mg, 0.32 mmol) and DMEDA (0.1 mL, 0.96 mmol) were added. The reaction was heated to 80 °C for 6 hours. The reaction was quenched with water and extracted with ethyl acetate. The organic layer was washed with brine and dried over MgSO₄. The solvent was evaporated under reduced pressure. The crude residue was purified with column chromatography (silica gel, hexanes/ethyl acetate 80:20) to give 2-(4-azidophenyl)-3-(1-(4-azidophenyl)-2-nitroethyl)-1H-indole as beige solid (513 mg, yield: 80%). MS: m/z=424.2 [M+H]⁺

### 2-(4-isothiocyanatophenyl)-3-(1-(4-isothiocyanatophenyl)-2-nitroethyl)-1H-indole

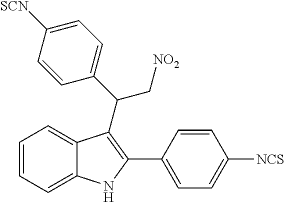

In a 250 mL round bottom flask 2-(4-azidophenyl)-3-(1-(4-azidophenyl)-2-nitroethyl)-1H-indole (500 mg, 1.18 mmol) was dissolved in anhydrous tetrahydrofuran (22 mL) and triphenylphosphine (1.6 mg, 6.15 mmol) and CS₂ (2.2 mL, 36.9 mmol) were added. The reaction was left stirring under an argon atmosphere at room temperature overnight. After completion, the reaction was quenched with water and extracted with ethyl acetate. The organic layer was washed with brine and dried over MgSO₄, and the solvents were evaporated under reduced pressure. The crude residue was purified with column chromatography (silica gel, hexane/ethyl acetate 80:20) to give 3-(12-(4-isothiocyanatophenyl)-3-(1-(4-isothiocyanatophenyl)-2-nitroethyl)-1H-indole (Compound 6) as a yellow solid (318 mg, yield 65%), MS: m/z=456.1 [M+H]⁺

### General Method F

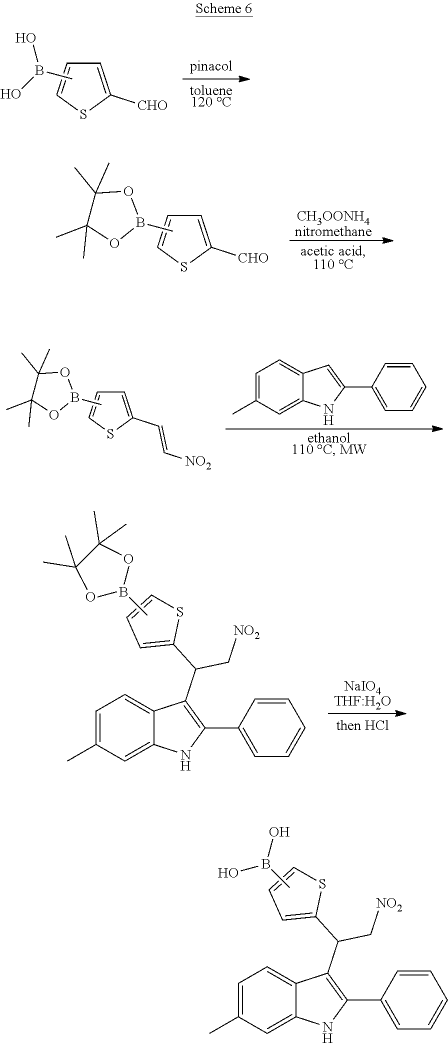

### Example 12. (5-(1-(6-methyl-2-phenyl-1H-indol-3-yl)-2-nitroethyl)thiophen-3-yl)boronic acid

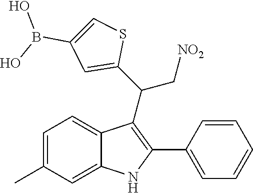 [SMILES: Cc1ccc2c(C(C[N+](=O)[O-])c3cc(B(O)O)cs3)c(-c3ccccc3)[nH]c2c1]

### 4-(4,4,5,5-tetramethyl-1,3,2-dioxaborolan-2-yl)thiophene-2-carbaldehyde

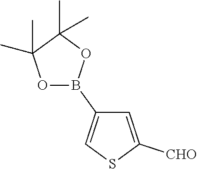 [SMILES: CC1(C)OB(c2csc(C=O)c2)OC1(C)C]

In a 250 mL round bottom flask, (5-formylthiophen-3-yl)boronic acid (1.2 g, 7.7 mmol) and pinacol (997 mg 8.5 mmol) were dissolved in anhydrous toluene (100 mL) under an argon atmosphere. The reaction mixture was refluxed for 14 hours at 120 °C. Toluene was evaporated under reduced pressure and the crude residue was purified with column chromatography (silica gel, hexanes/ethyl acetate 95:5 to 90:10) to give pure 4-(4,4,5,5-tetramethyl-1,3,2-dioxaborolan-2-yl)thiophene-2-carbaldehyde as a white solid (1.66 g, yield: 90%).

### (E)-4,4,5,5-tetramethyl-2-(5-(2-nitrovinyl)thiophen-3-yl)-1,3,2-dioxaborolane

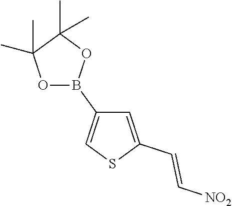 [SMILES: CC1(C)OB(c2csc(/C=C/[N+](=O)[O-])c2)OC1(C)C]

In a 100 mL round bottom flask 4-(4,4,5,5-tetramethyl-1,3,2-dioxaborolan-2-yl)thiophene-2-carbaldehyde (1.25 g, 6.3 mmol), nitromethane (1.7 mL, 31.4 mmol) and ammonium acetate (2.07 g, 25.4 mmol) were dissolved in acetic acid (13 mL) and the reaction mixture was heated at 110 °C under an argon atmosphere for 7 hours. The reaction was then quenched with ice water and extracted with methylene chloride. The organic layer was dried over anhydrous MgSO₄, and the solvent was evaporated under reduced pressure. The crude residue was purified with column chromatography (silica gel, hexanes/diethyl ether 90:10) to give (E)-4,4,5,5-tetramethyl-2-(5-(2-nitrovinyl)thiophen-3-yl)-1,3,2-dioxaborolane as yellow solid (1.2 g, yield: 67%).

### 6-methyl-3-(2-nitro-1-(4-(4,4,5,5-tetramethyl-1,3,2-dioxaborolan-2-yl)thiophen-2-yl)ethyl)-2-phenyl-1H-indole

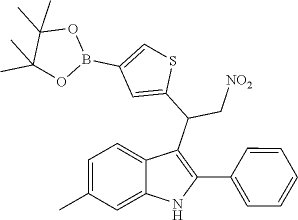 [SMILES: Cc1ccc2c(C(C[N+](=O)[O-])c3cc(B4OC(C)(C)C(C)(C)O4)cs3)c(-c3ccccc3)[nH]c2c1]

In a 20 mL microwave tube (E)-4,4,5,5-tetramethyl-2-(5-(2-nitrovinyl)thiophen-3-yl)-1,3,2-dioxaborolane (1 g, 3.56 mmol) and 6-methyl-2-phenyl-1H-indole (736 mg, 3.56 mmol) were dispersed in anhydrous ethanol. The tube was sealed, and the reaction mixture was put under microwave irradiation at 100 °C for 45 minutes. After completion of the reaction, ethanol was evaporated under reduced pressure and the crude residue was purified with column chromatography (silica gel, hexanes/diethyl acetate 80:20) to give 6-methyl-3-(2-nitro-1-(4-(4,4,5,5-tetramethyl-1,3,2-dioxaborolan-2-yl)thiophen-2-yl)ethyl)-2-phenyl-1H-indole as a white solid (955 mg, yield: 55%). MS: m/z=488.1 [M+H]⁺

### (5-(1-(6-methyl-2-phenyl-1H-indol-3-yl)-2-nitroethyl)thiophen-3-yl)boronic acid

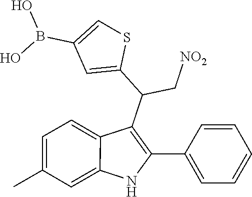 [SMILES: Cc1ccc2c(C(C[N+](=O)[O-])c3cc(B(O)O)cs3)c(-c3ccccc3)[nH]c2c1]

In a 50 mL round bottom flask, 6-methyl-3-(2-nitro-1-(4-(4,4,5,5-tetramethyl-1,3,2-dioxaborolan-2-yl)thiophen-2-yl)ethyl)-2-phenyl-1H-indole (400 mg, 0.8 mmol) and NaIO₄ (495 mg, 2.3 mmol) were dissolved in a mixture of THF:H₂O (7.4 ml: 2.1 ml) and left stirring at room temperature for 30 minutes. Then, aqueous hydrochloric acid solution 1M (0.55 mL, 0.55 mmol) was added and the reaction was stirred at room temperature for additional 17 hours. After completion, the reaction was quenched with water and extracted with methylene chloride. The organic layer was washed with brine, dried over anhydrous MgSO₄ and the solvent was evaporated under reduced pressure. The crude residue was purified with column chromatography (silica gel, hexanes/ethyl acetate 80:20 to 30:70) to give (5-(1-(6-methyl-2-phenyl-1H-indol-3-yl)-2-nitroethyl)thiophen-3-yl)boronic acid as white solid (212 mg, yield: 64%). MS: m/z=406.5 [M+H]⁺.

### In Vitro Studies

Cell membrane preparation. Fresh or frozen HEK293 cell pellets (2×10⁸ cells expressing human CB1 (hCB1) or rat CB1 (rCB1)) were resuspended in 5 ml of TME [25 mM Tris, 5 mM MgCl2, 1 mM EDTA pH 7.4] supplemented with 0.5% protease inhibitor cocktail (Sigma, St Louis, MO, USA). Cells were lysed under nitrogen cavitation for 30 min at 1000 psi on ice, centrifuged at 1000 g for 10 min, and the supernatant was collected. The pellet was washed three times with 10 ml of TME containing protease inhibitors and the combined supernatant was centrifuged at 100,000 g for 45 min at 4 °C. The resultant membrane pellet was used immediately or stored at −80 °C. Protein concentration for the resulting membrane fraction was obtained using Bradford Assay.

Competition Binding Assay. Competition binding assays were performed in a 96-well format. Assays were carried out using [3H] CP55,940 and Tris binding buffer (50 mM Tris-HCl, 50 mM Tris-base, 0.1% BSA, pH 7.4), up to a total assay volume of 500 μL. The concentration of [³H]CP55,940 used in our displacement assays was 0.7 nM. The test compounds were stored as 10 mM stock solutions in DMSO, and prepared for the assay with serial dilutions in concentrations of 0.023 nM to 2.27 μM with the vehicle concentration in all assay wells being 0.1% DMSO. Binding was initiated by the addition of transfected hCB1 HEK293 cell membranes (50 μg of protein per well). All assays were performed at 37 °C for 60 min before termination by the addition of ice-cold Tris binding buffer, followed by vacuum filtration using a 24-well sampling manifold (Brandel Cell Harvester; Brandel Inc., Gaithersburg, MD) and Brandel GF/B filters that had been soaked in wash buffer at 4 °C for at least 24 h. Each reaction well was washed six times with a 1.2 mL aliquot of Tris binding buffer. The filters were oven-dried for 60 min and then placed in 3 mL of scintillation fluid (Ultima Gold XR, PerkinElmer, Seer Green, Buckinghamshire, UK). Radioactivity was quantified by liquid scintillation spectrometry. Specific binding was defined as the difference between the binding that occurred in the presence and absence of 1 μM unlabeled CP55,940. Ki values were determined by fitting the data to non-linear regression curve using Graph Pad Prism software. For example, compounds 1, 4 and 7 (up to 100 nM) do not compete with CP-55,940 unlike typical CB1 orthosteric compounds.

Saturation Binding Assay. Saturation-binding assays were performed in a 96-well format. Membrane preparations either from rat brain or HEK293 cells overexpressing hCB1 were resuspended in TME-bovine serum albumin (BSA; TME containing 0.1% BSA) and 25 ug of protein was added to each assay well. [³H]CP55940 was diluted in TME-BSA to yield ligand concentrations ranging from 1.58 to 50 nM. Test compounds were dissolved in DMSO and added to the well at a specific predefined concentration. Nonspecific binding was assayed in the presence of 5 μM unlabeled CP55940. The assay was performed at 30 °C for 1 h with gentle agitation. The resultant material was transferred to Unifilter GF/B filter plates and unbound ligand removed using a Packard Filtermate-96 Cell Harvester (Perkin-Elmer Packard, Shelton, CT, USA). Filter plates were washed four times with ice-cold wash buffer (50 mM Tris base, 5 mM MgCl₂ containing 0.5% BSA, pH 7.4). Bound radioactivity was quantitated in a Packard Top Count Scintillation Counter. Nonspecific binding was subtracted from the total bound radioactivity to calculate specific binding of [³H] CP55940 (represented as pmol/mg protein). All assays were performed in triplicate and data points presented as the mean. Bmax and Kd values were calculated by nonlinear regression using Graph Pad Software (one site-binding analysis equation Y·Bmax·X/(Kd+X). For example, compound 7 showed a 45% increase in the Bmax of CP-55,940.

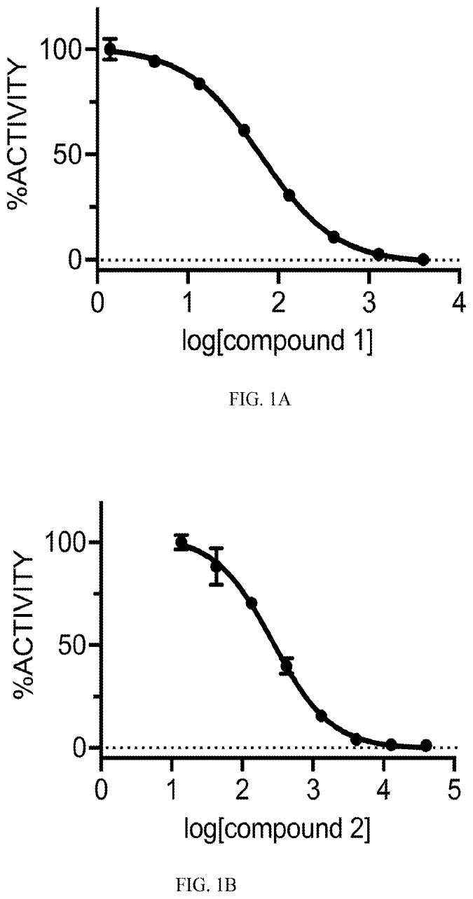

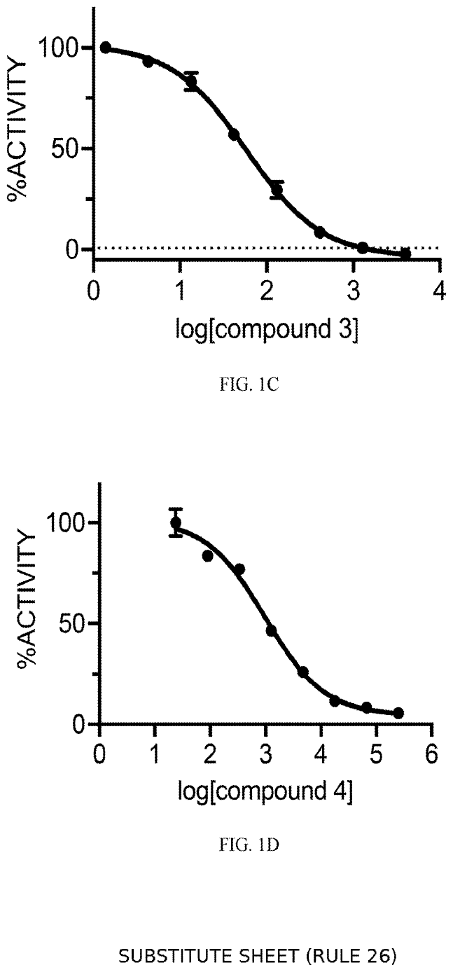

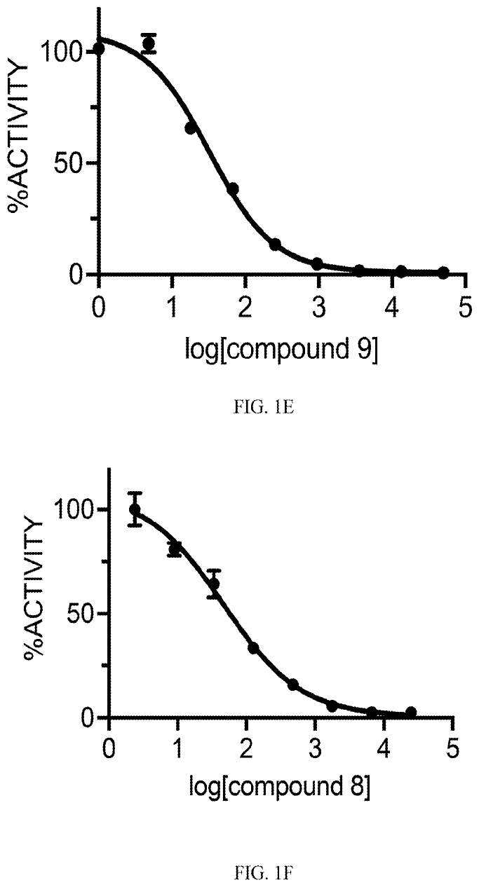

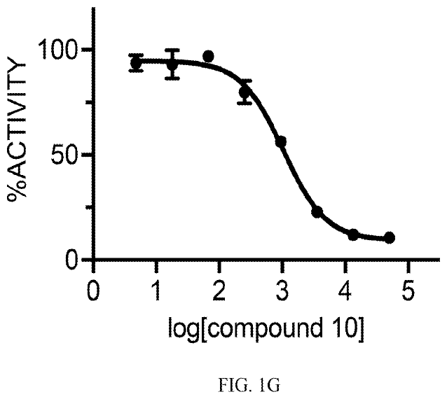

Fluorescent assay protocol for human MGL. Compound inhibition of hMGL activity was assessed by a fluorometric assay. The assay was carried out in a 96-well plate format and hMGL was monitored by the hydrolysis of the substrate 7-hydroxy-6-methoxy-4-methyl-coumarin ester (AHMMCE) or the substrate valeroyl-7-hydroxy-6-methoxy-4-methylcoumarin ester (VHMMCE) to form the fluorescent product, coumarin. Briefly, various concentrations of each compound were preincubated with hMGL (175 ng of total protein in E. coli lysate containing hMGL) for 15 minutes at room temperature. Upon the addition of the substrate, the reaction was incubated at 250 °C for 120 minutes; fluorescence readings were taken every 15 minutes at 360 nm/460 nm (2excitation/λmission) using a Synergy HT Plate Reader (Bio-Tek, Winooski, Vt.). Under the incubation conditions, negligible substrate hydrolysis was observed. External standards were used to convert relative fluorescence units to the amount of 4-methylcoumarin formed. hMGL assays were performed in triplicate for each inhibitor concentration, and IC₅₀ values were determined using Prizm software (FIGS. 1A-1G). For example, Compounds 1-3, 8-9 inhibited hMGL with IC₅₀ values 63.5 nM, 270.9 nM, 58.8 nM, 46.8 nM and 32.3 nM respectively. In comparison, known commercial ligands ZCZ011 (CAS registry no. 1998197-39-9; 6-methyl-3-(2-nitro-1-(thiophen-2-yl)ethyl)-2-phenyl-1H-indole) and GAT211 (F-0870-0064, F-0870, AZ-4; CAS registry no. 102704-40-5; 3-(2-nitro-1-phenylethyl)-2-phenyl-1H-indole) did not show any hMGL inhibition at concentrations up to 10 μM.

| | MGL |
|---|---|
| | inhibition IC₅₀ |
| Example | (nM) |
| Compound 1 | 63.5 |
| Compound 2 | 270.9 |
| Compound 3 | 58.8 |
| Compound 4 | 979.2 |
| Compound 8 | 46.8 |
| Compound 9 | 32.3 |
| Compound 10 | 1159 |

Fluorescent assay protocol for hFAAH. Procedure was followed as described for hMGL, except that arachidonoyl-methylcoumarin was used as the fluorogenic substrate. Compounds were diluted in 50:50 DMSO-assay buffer (50 mM HEPES, 1 mM EDTA, 0.1% BSA, pH 7.4) so as to have a final DMSO concentration below 8% in each reaction. The test compounds, 15 jag of hFAAH and assay buffer were pre-incubated for 15 min at 250 °C. The substrate (20 μM) was added before the incubation at 250 °C. Kinetic fluorescence reading was obtained every 20 minutes (λex=360/λem=460) for 4 hours on a BioTek Synergy HT Microplate Reader. The fluorescence reading at the 3 hour time point (linear enzyme kinetics) was used to calculate percent inhibition based on control assays without inhibitor present. FAAH assays were performed in triplicate for each inhibitor concentration, and IC₅₀ values were determined using Prizm software (GraphPad Software, Inc.). For example, some compounds disclosed herein inhibited hFAAH with IC₅₀ values ranging from 1 nM-100 μM.

Fluorescent assay protocol for hABHD6. In each well of a 96-well plate 8 μl of membrane fraction containing full-length hABHD6 (1 μg total protein) was mixed with 168 μL of assay buffer (50 mM Tris-HCl, pH 7.6) and 20 μl of the test compounds diluted in 50:50 DMSO: assay buffer. Each plate was incubated at rt for 15 minutes before adding 4 μL of 1 mM 7-hydroxy-6-methoxy-4-methyl-coumarin ester (AHMMCE) or valeroyl-7-hydroxy-6-methoxy4-methylcoumarin ester (VHMMCE) substrate. The reaction was allowed to proceed for 1 hour at rt before the fluorescence was read (λex=360/λem=460). hABHD6 assays were performed in triplicate for each inhibitor concentration, and IC₅₀ values were determined using Prizm software (GraphPad Software, Inc.). For example, some compounds inhibited hABHD6 with IC₅₀ values ranging from 1 nM-100 μM.

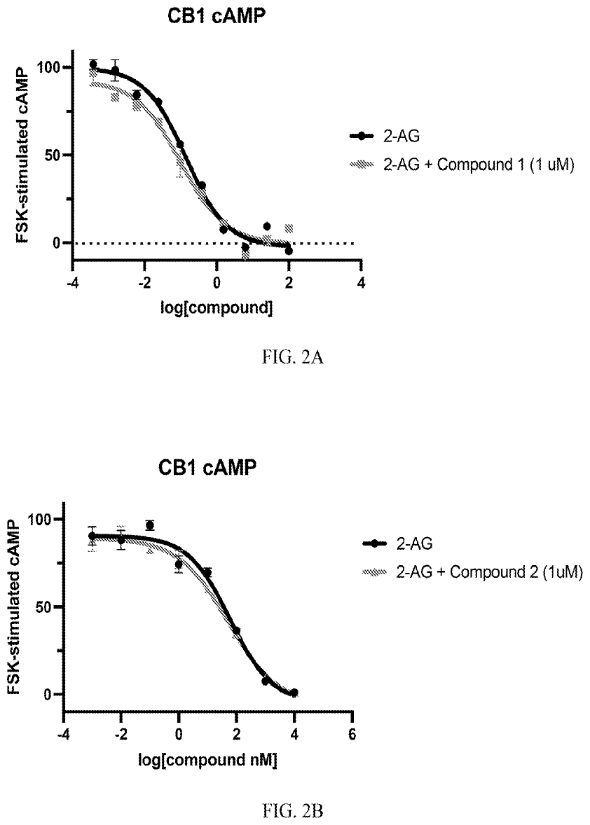

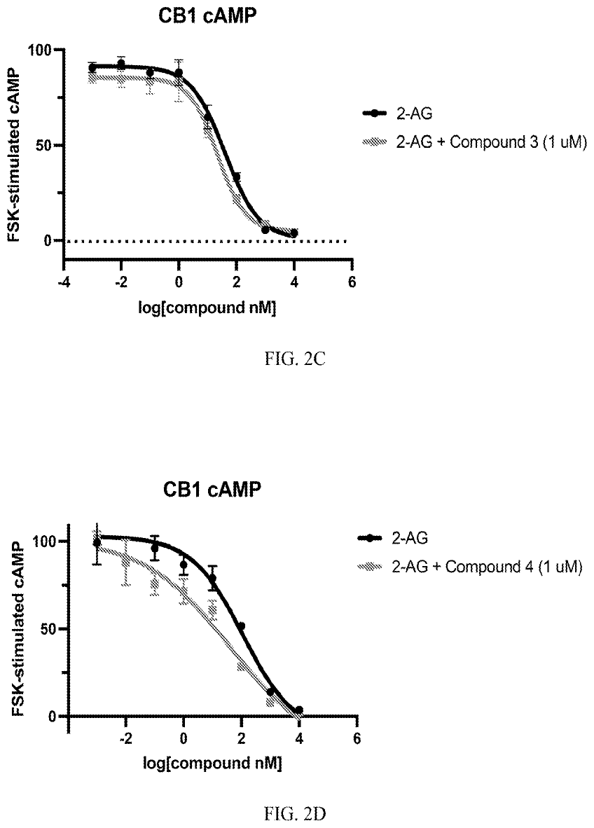

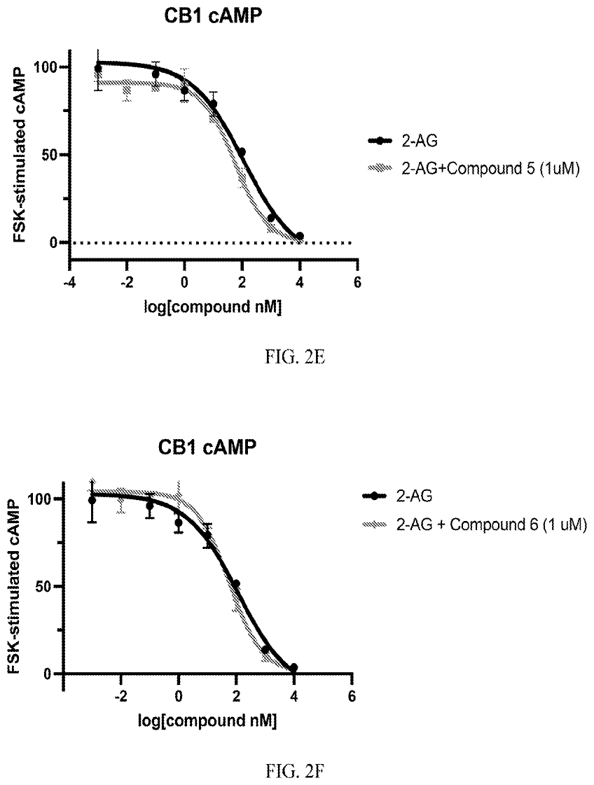

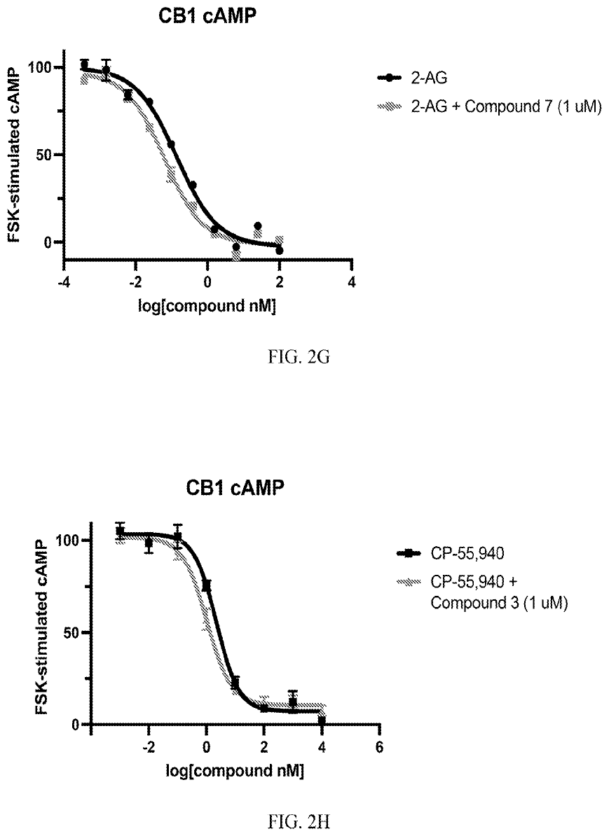

CB1 cAMP assays. Compounds are evaluated for their ability to modulate the actions of orthosteric ligands at CB1 receptor sites. HEK293 and/or CHO cells stably expressing hCB1 receptors were used for the studies. The CAMP assay was carried out using Perkin Elmer's Lance Ultra cAMP kit following the protocol as described in J Biomol Screen 1999, 4, (6), 303-308. The assays are carried out in a 384-well format using 1000-1500 cells/well. The cells are harvested with non-enzymatic cell dissociation reagent Versene, washed once with Hanks' Balanced Salt Solution (HBSS), and resuspended in the stimulation buffer. The test compounds (5 μL, 1 or 10 μM final concentration) containing forskolin (2 μM final concentration), stimulation buffer, and various concentration of the orthosteric agonist (0.01 nM to 10 μM) that was either CP-55,940, 2-AG or AEA are added to the plate followed by cell suspension (5 μL). After 30 minutes of stimulation at room temperature, the Eu-CAMP tracer working solution (5 μL) and Ulight-anti-cAMP working solution (5 μL) are added to the plate and incubated at room temperature for 60 minutes. The data are collected via PerkinElmer Envision plate reader. The EC₅₀ values are determined by nonlinear regression analysis using GraphPad Prism software (FIGS. 2A-2H). For example, compound 3 behaves as a positive allosteric modulator of both 2-AG and CP-55,940 at the CB1 receptor as it increases the potency of the orthosteric ligands by ~2-fold. A similar effect is also shown by compounds 4-7 for the enhancement of 2-AG EC₅₀.

EC₅₀ (nM)

| Example | 2-AG | 2-AG + Compound (1 μM) |
|---|---|---|
| Compound 1 | 139.7 | 101.9 |
| Compound 2 | 60.9 | 51.3 |
| Compound 3 | 60.9 | 37.5 |
| Compound 4 | 114.5 | 40.8 |
| Compound 5 | 114.5 | 57.0 |
| Compound 6 | 114.5 | 57.2 |
| Compound 7 | 139.7 | 66.4 |

[³⁵S]GTPYS Binding to CBIR in Mouse Striatal Membranes. To assess G protein coupling in mouse brain, striata from C57BL/6J mice (4-7 months old) were collected, minced, and disrupted in a glass homogenizer in homogenization buffer (10 mM Tris-HCl, pH 7.4, 100 mM NaCl, 1 mM EDTA, 1 mM DTT). The homogenate was passed through a 26-gauge needle, centrifuged twice at 20000 g for 30 min at 4 °C, and resuspended in assay buffer (50 mM Tris-HCl pH 7.4, 100 mM NaCl, 5 mM MgCl2, 1 mM EDTA, 20 μM GDP, 1 mM DTT). For each reaction, 2.5 μg of membrane protein was incubated in assay buffer containing ~0.1 nM [35S]GTPγS, and increasing concentrations of the test compounds (0.1 nM-10 μM) or increasing concentrations of CB1 agonists (CP-55,940, 2-AG or AEA) co-incubated with defined concentrations of the test compounds in a total volume of 200 μL for 2 h at room temperature. Test compounds were diluted through serial dilutions in DMSO and then with assay buffer to a final DMSO concentration of less than 1%. Reactions were terminated by separating membrane-bound and free [35S]GTPγS through filtration with GF/B filters using a 96-well plate harvester (Brandel Inc., Gaithersburg, MD). Filters were dried overnight, and radioactivity was determined with a microplate scintillation counter. Data were fit by nonlinear regression using GraphPad Prism and the four-parameter logistic dose-response curve: response=bottom+((top−bottom)/(1+10 (log EC₅₀−log X))). For example, some compounds disclosed herein showed significant enhancement of both Eₘₐₓ and EC₅₀ of 2-AG.

βArrestin2 Assay. βArrestin2 recruitment was determined using the CHO-hCB1R PathHunter assay (DiscoveRx) according to the manufacturer's instructions. Briefly, cells (20 000 cells/well in low-volume 96-well plates) were incubated overnight in Opti-MEM (Invitrogen) containing 1% FBS at 37 °C and under a 5% CO₂ atmosphere. Following this, cells were co-treated at 37 °C with test ligands (alone or in the presence of increasing concentrations of CB1 agonists (CP-55,940, 2-AG or AEA) for 90 min. The detection solution was then added to cells according to the manufacturer's directions, and cells were incubated for 60 min at room temperature. Chemiluminescence was measured on a Cytation 5 plate reader (top read; gain, 200; integration time, 10 000 ms). All experiments included a vehicle control and the response was normalized to the percent of response produced by the full agonist CP55,940. For example, some compounds disclosed herein did not show a significant enhancement of 2-AG potency in recruiting β-arrestin when added with 2-AG at a concentration of 10 μM.

### In Vivo Studies

The CFA-Induced Inflammatory pain and Paclitaxel-induced neuropathic pain models are used to assess the ability of the test compounds to suppress inflammatory and neuropathic pain, respectively.

CFA-Induced Inflammatory Nociception. Mice were administered an intraplantar injection (20 μL) of Complete Freund's adjuvant (CFA) (diluted 1:1 in saline) in the right hind paw with a 28.5-gauge needle. After approximately 48 hours, escalating doses of the test compounds were administered i.p. Mechanical thresholds that elicited paw withdrawal were assessed with an electronic von Frey anesthesiometer (IITC Life Science, Woodland Hills, CA) 30 minutes after i.p administration of the test compounds. Thresholds were also assessed prior to CFA injection (baseline) and 48 h after CFA administration (pre-drug baseline)

Paclitaxel-Induced Neuropathic Pain. Paclitaxel (4 mg/kg i.p.) was dissolved in vehicle containing 5% cremophor-EL (Sigma-Aldrich), 5% ethanol, 90% saline. Paclitaxel or cremophor-based vehicle was injected i.p. on days 0, 2, 4, and 6. During the maintenance phase of paclitaxel-induced neuropathy (on day 15 after paclitaxel administration), test compounds (0.1, 0.3, 1, 3, 10, and 30 mg/kg) were administered i.p. The effects (mechanical and cold allodynia) of pharmacological manipulations were assessed 30 min after drug administration.

Assessment of Mechanical Allodynia: Paw withdrawal thresholds (g) to mechanical stimulation were measured using an electronic von Frey anesthesiometer supplied with a 90-gram range probe (IITC Life Science Inc., CA, USA). Mice were placed in transparent plastic chambers on an elevated metal mesh table and were habituated to the testing apparatus for 30 min until exploratory behavior had ceased. Then, a force was applied to the midplantar region of the hind paw by a semi-flexible tip connected to the anesthesiometer. Mechanical stimulation was terminated when the mouse withdrew its paw. The value of the applied force (in g) was measured in duplicate for each paw and reported as the mean obtained from each animal averaged across paws.

For example, some compounds disclosed herein suppressed mechanical allodynia in paclitaxel-treated mice with ED₅₀ values ranging from 0.1-30 mg/kg.

Time course studies: The effect of the test compounds to suppress mechanical allodynia in paclitaxel-treated mice was also tested in a time course study. The methodology followed was the same as the one used for the dose-response study. The compounds were administered at a dose of 30 mg/kg and their effects were monitored over time (0.5, 2, 4, 6 and 24 hrs).

For example, some compounds disclosed herein (single dose) showed sustained effects for 4-12 hours.

Assessment of Cold Allodynia: The duration of time(s) spent attending to acetone-stimulated paws was used to assess cold allodynia. Mice were individually placed underneath transparent plastic chambers on an elevated metal mesh table. After a 30-min habituation period, an acetone bubble (approximately 20 μl) that formed at the end of a blunt 1 ml syringe was applied to the plantar surface of the hind paw. The time that the animal spent attending to the acetone-stimulated paw (i.e. elevation, shaking, or licking) was recorded over a 60s observation period. Three measurements were taken for each paw alternately with a 5 min interval between applications. Cold response time(s) for each animal was determined by averaging the duration of time spent responding to acetone across the six acetone applications.

For example, some compounds disclosed herein dose-dependently reduced paclitaxel-induced hypersensitivity to mechanical and cold stimulation with ED₅₀ values ranging from 1-50 mg/kg i.p. For example, some compounds disclosed herein dose-dependently reduced paclitaxel-induced hypersensitivity to mechanical and cold stimulation with ED₅₀ values ranging from 0.1-30 mg/kg i.p.

Tolerance studies in Comparison to Orthosteric Cannabinoid Agonist. The development of tolerance was assessed using the paclitaxel-induced peripheral neuropathy pain model. Test compounds (0.1-30 mg/kg/day, i.p 14 days) or vehicle were administered once daily to naïve and paclitaxel-treated mice. Separate naive groups received the orthosteric cannabinoid agonist WIN55,212-2 (3 mg/kg/day i.p. 14 days). Responses to mechanical stimulation were recorded before and 30 minutes following pharmacological manipulations. For example, some compounds disclosed herein showed sustained efficacy in suppressing paclitaxel-induced mechanical allodynia with no signs of tolerance at several doses tested (0.1-30 mg/kg, i.p. 14 days).

Pharmacological Specificity. The pharmacological specificity assays were performed to evaluate if the anti-nociceptive effects of the developed ligands are CB1 mediated. Test compounds (0.1-30 mg/kg i.p.) were administered alone or co-administered with either the CB1 antagonist AM251 (5 mg/kg i.p.) or the CB2 antagonist AM630 (5 mg/kg i.p.). Responses to mechanical stimulation were recorded before and 30 minutes following pharmacological manipulations. For example, antiallodynic efficacy of some compounds disclosed herein was reversed by AM251 but not from AM630, thus indicating that the effect is CB1-mediated.

Aqueous solubility assays. The kinetic aqueous solubility of the compounds is determined using laser-based nephelometry. Linear serial dilutions of each compound are prepared in PBS buffer (pH=7.4) from DMSO stock solution (10 mM) in a 384-well plate. The compound solutions are mixed and incubated for 120 minutes at room temperature, followed by approximating the precipitation points for each compound using light scattering (laser-based BMG Labtech NEPHELOstar PLUS microplate nephelometer). In some aspects, the final solutions may contain 0 mM, 55 mM, 135 mM, or 500 mM of mannitol. For example, (4-(2-nitro-1-(2-phenyl-1H-indol-3-yl)ethyl)phenyl)boronic acid (Compound 1) displays a significantly higher aqueous solubility (>100 fold) compared to the known commercial ligand ZCZ011 (CAS registry no. 1998197-39-9; 6-methyl-3-(2-nitro-1-(thiophen-2-yl)ethyl)-2-phenyl-1H-indole).

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

1. A compound of formula I, 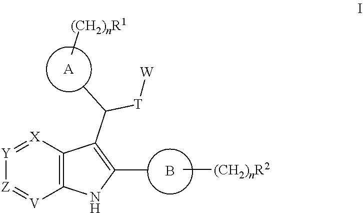
and their pharmaceutically acceptable salts, solvates, hydrates, polymorphs, enantiomers, diastereomers, geometric isomers, racemates, tautomers, rotamers, atropisomers, isotopic variations, or N-oxides thereof,
wherein:
Ring A is C5-10 aryl, a 3-7 membered monocyclic heteroaryl ring having 1-4 heteroatoms independently selected from nitrogen, oxygen, or sulfur, a 3-8 membered saturated or partially unsaturated carbocyclic ring, a 3-7 membered saturated or partially unsaturated heterocyclic ring having 1-4 heteroatoms independently selected from nitrogen, oxygen, phosphorus, boron, or sulfur; each of which is optionally substituted;
Ring B is C5-10 aryl, a 3-7 membered monocyclic heteroaryl ring having 1-4 heteroatoms independently selected from nitrogen, oxygen, or sulfur, a 3-8 membered saturated or partially unsaturated carbocyclic ring, a 3-7 membered saturated or partially unsaturated heterocyclic ring having 1-4 heteroatoms independently selected from nitrogen, oxygen, phosphorus, boron, or sulfur; each of which is optionally substituted;
X, Y, Z, V are independently selected from nitrogen, —C((CH₂)ₙR₃)—, or —C(R₄)—;
R₁, R₂, R₃, and R₄ are independently selected from hydrogen, —B(OH)₂, —B(OR₅)₂, —B(OH)(OR₆), —B(OR₇)₂NR₈, BF₃, provided that at least one of them is —B(OH)₂, —B(OR₅)₂, —B(OH)(OR₆), —B(OR₇)₂NR₈, or BF₃;
each n is independently selected from 0, 1, 2, 3 or 4; as used in this disclosure when an integer such as n is 0 the structural portion modified by that integer is absent and the adjacent subunits are directly connected;
each R₁, R₂, R₃, and R₄ are independently selected from hydrogen, deuterium, F, Cl, Br, I, C≡N, N₃, NCS, —S(═O)₂F, —OS(═O)₂F, —R, —OR, —SR, —C(═O)R, —OC(═O)R, —S(═O)₂R, —S(═O)R, —N(R)S(═O)₂R, —S(═O)₂NR₂, —C(═O)OR, —C(═O)NR₂, —N(R)C(═O)R, —N(R)C(═O)NR₂, —C(═O)N(R)C(═O)R, —NR₂, optionally substituted C₅₋₁₀ aryl, optionally substituted C5-10 heteroaryl having 1-4 heteroatoms independently selected from nitrogen, oxygen, or sulfur, optionally substituted C₃₋₈ saturated or partially unsaturated carbocyclic ring, optionally substituted C₃₋₈ saturated or partially unsaturated heterocyclic ring having 1-4 heteroatoms independently selected from nitrogen, oxygen, or sulfur;
R₅ is independently selected from C₁₋₄ alkyl, C₁₋₄ alkenyl, C₁₋₄ alkynyl, C₃₋₆ cycloalkyl; each of which is optionally substituted; two independent occurrences of R⁵, taken together with their intervening atom(s), form an optionally substituted 4-8-membered saturated or partially unsaturated cyclic ring having 0-4 heteroatoms independently selected from nitrogen, oxygen, boron, or sulfur;
R₆ is independently selected from C₁₋₄ alkyl, C₁₋₄ alkenyl, C₁₋₄ alkynyl, C₃₋₆ cycloalkyl; each of which is optionally substituted; or forms an optionally substituted 4-8 membered saturated or partially saturated heterocyclic ring with an atom of Ring A, Ring B, or the indole core having 0-4 heteroatoms independently selected from nitrogen, oxygen, or boron;
R₇ is independently selected from C₁₋₄ alkyl, C₁₋₄ alkenyl, C₁₋₄ alkynyl, C₃₋₆ cycloalkyl; each of which is optionally substituted; each independent occurrence of R₇, taken together with its intervening atom(s), boron, and nitrogen, forms an optionally substituted 4-8-membered saturated or partially unsaturated cyclic ring having 0-4 heteroatoms independently selected from nitrogen, oxygen, boron, or sulfur;
R₈ is C₁₋₄ alkyl, C₁₋₄ alkenyl, C₁₋₄ alkynyl, C₃₋₆ cycloalkyl; each of which is optionally substituted;
each R is independently hydrogen, C₁₋₆ aliphatic, C₅₋₁₀ aryl, a 3-7 membered monocyclic heteroaryl ring having 1-4 heteroatoms independently selected from nitrogen, oxygen, or sulfur, a 3-8 membered saturated or partially unsaturated carbocyclic ring, a 3-7 membered saturated or partially unsaturated heterocyclic ring having 1-4 heteroatoms independently selected from nitrogen, oxygen, or sulfur; each of which is optionally substituted;
T is C₁₋₄ alkylene, C₁₋₄ alkenylene, C₁₋₄ alkynylene, C3-6 cycloalkylene; each of which is optionally substituted;
W is selected from —NO₂, —CF₃, —C≡N, NH₂, NHC(═O) CH₂-halogen, —ONO₂, —S(═O)₂F, —OS(═O)₂F, —OH, —C(═O) OH, —B(OH)₂, or —SF₅.

2. The compound of claim 1, wherein R¹ is —B(OH)₂.

3. The compound of claim 1, wherein W is NO₂, or CF₃.

4. The compound of claim 1, wherein Ring A is phenyl or C₅₋₆ heteroaryl having 1-2 heteroatoms independently selected from nitrogen, oxygen, or sulfur; and Ring B is phenyl or C₅₋₆ heteroaryl having 1-2 heteroatoms independently selected from nitrogen, oxygen, or sulfur.

5. (canceled)

6. The compound of claim 1, selected from
3-(2-nitro-1-(4-(4,4,5,5-tetramethyl-1,3,2-dioxaborolan-2-yl)phenyl)ethyl)-2-phenyl-1H-indole;
(4-(2-nitro-1-(2-phenyl-1H-indol-3-yl)ethyl)phenyl)boronic acid;
3-(2-nitro-1-(3-(4,4,5,5-tetramethyl-1,3,2-dioxaborolan-2-yl)phenyl)ethyl)-2-phenyl-1H-indole;
(3-(2-nitro-1-(2-phenyl-1H-indol-3-yl)ethyl)phenyl)boronic acid;
(R)-(4-(2-nitro-1-(2-phenyl-1H-indol-3-yl)ethyl)phenyl)boronic acid;
(S)-(4-(2-nitro-1-(2-phenyl-1H-indol-3-yl)ethyl)phenyl)boronic acid;
3-(2-nitro-1-phenylethyl)-2-(4-(4,4,5,5-tetramethyl-1,3,2-dioxaborolan-2-yl)phenyl)-1H-indole;
(4-(3-(2-nitro-1-phenylethyl)-1H-indol-2-yl)phenyl)boronic acid;
2-(4-azidophenyl)-3-(2-nitro-1-(4-(4,4,5,5-tetramethyl-1,3,2-dioxaborolan-2-yl)phenyl)ethyl)-1H-indole;
(4-(1-(2-(4-azidophenyl)-1H-indol-3-yl)-2-nitroethyl)phenyl)boronic acid;
(4-(1-(2-(4-isothiocyanatophenyl)-1H-indol-3-yl)-2-nitroethyl)phenyl)boronic acid;
3-(2-nitro-1-phenylethyl)-2-phenyl-6-(4,4,5,5-tetramethyl-1,3,2-dioxaborolan-2-yl)-1H-indole; or
(3-(2-nitro-1-phenylethyl)-2-phenyl-1H-indol-6-yl)boronic acid.

7. A compound of formula II, 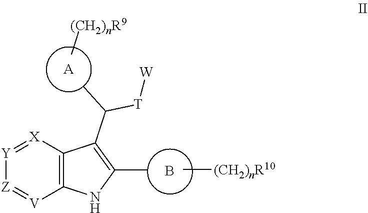
and their pharmaceutically acceptable salts, solvates, hydrates, polymorphs, enantiomers, diastereomers, geometric isomers, racemates, tautomers, rotamers, atropisomers, isotopic variations, or N-oxides thereof,
wherein:
Ring A is C₅₋₁₀ aryl, a 3-7 membered monocyclic heteroaryl ring having 1-4 heteroatoms independently selected from nitrogen, oxygen, or sulfur, a 3-8 membered saturated or partially unsaturated carbocyclic ring, a 3-7 membered saturated or partially unsaturated heterocyclic ring having 1-4 heteroatoms independently selected from nitrogen, oxygen, phosphorus, boron, or sulfur; each of which is optionally substituted;
Ring B is C₅₋₁₀ aryl, a 3-7 membered monocyclic heteroaryl ring having 1-4 heteroatoms independently selected from nitrogen, oxygen, or sulfur, a 3-8 membered saturated or partially unsaturated carbocyclic ring, a 3-7 membered saturated or partially unsaturated heterocyclic ring having 1-4 heteroatoms independently selected from nitrogen, oxygen, phosphorus, boron, or sulfur; each of which is optionally substituted;
X, Y, Z, V are independently selected from nitrogen, —C((CH₂)ₙR₁₁)—, or —C(R¹²)—;
R₉, R₁₀, R₁₁, and R₁₂ are independently selected from hydrogen, NCS, S(═O)₂F, NHC(═O)C₂₋₄ alkenyl, NHC(═O)CH₂-halogen, or N₃ provided that at least one of them is —NCS, S(═O)₂F, NHC(═O)C₂₋₄ alkenyl, NHC(═O)CH₂-halogen, or N₃;
each n is independently selected from 0, 1, 2, 3 or 4; as used in this disclosure when an integer such as n is 0 the structural portion modified by that integer is absent and the adjacent subunits are directly connected;
each R⁹, R¹⁰, R¹¹, and R¹² are independently selected from hydrogen, deuterium, F, Cl, Br, I, C≡N, N₃, NCS, —S(═O)₂F, —OS(═O)₂F, —R, —OR, —SR, —C(═O)R, —OC(═O)R, —S(═O)₂R, —S(═O)R, —N(R) S(═O)₂R, —S(═O)₂NR₂, —C(═O) OR, —C(═O)NR₂, —N(R)C(═O)R, —N(R)C(═O)NR₂, —C(═O)N(R)C(═O)R, —NR₂, optionally substituted C₅₋₁₀ aryl, optionally substituted C₅₋₁₀ heteroaryl having 1-4 heteroatoms independently selected from nitrogen, oxygen, or sulfur, optionally substituted C₃₋₈ saturated or partially unsaturated carbocyclic ring, optionally substituted C₃₋₈ saturated or partially unsaturated heterocyclic ring having 1-4 heteroatoms independently selected from nitrogen, oxygen, or sulfur;
each R is independently hydrogen, C₁₋₆ aliphatic, C₅₋₁₀ aryl, a 3-7 membered monocyclic heteroaryl ring having 1-4 heteroatoms independently selected from nitrogen, oxygen, or sulfur, a 3-8 membered saturated or partially unsaturated carbocyclic ring, a 3-7 membered saturated or partially unsaturated heterocyclic ring having 1-4 heteroatoms independently selected from nitrogen, oxygen, or sulfur; each of which is optionally substituted;
T is C₁₋₄ alkylene, C₁₋₄ alkenylene, C₁₋₄ alkynylene, C₃₋₆ cycloalkylene; each of which is optionally substituted;
W is selected from —NO₂, —CF₃, —C≡N, NH₂, NHC(═O) CH₂-halogen, —ONO₂, —S(═O)₂F, —OS(═O)₂F, —OH, —C(═O)OH, or —SF₅.

8. The compound of claim 7, wherein R₉ is —NCS.

9. The compounds of claim 7, wherein R₁₀ is S(═O)₂F.

10. The compound of claim 7, wherein W is NO₂, or CF₃.

11. The compound of claim 7, wherein Ring A is phenyl or C₅₋₆ heteroaryl having 1-2 heteroatoms independently selected from nitrogen, oxygen, or sulfur; and Ring B is phenyl or C₅₋₆ heteroaryl having 1-2 heteroatoms independently selected from nitrogen, oxygen, or sulfur.

12. (canceled)

13. The compound of claim 7, selected from
2-(4-isothiocyanatophenyl)-3-(1-(4-isothiocyanatophenyl)-2-nitroethyl)-1H-indole;
3-(1-(4-isothiocyanatophenyl)-2-nitroethyl)-2-phenyl-1H-indole; or
4-(3-(2-nitro-1-phenylethyl)-1H-indol-2-yl)benzenesulfonyl fluoride.

14. (canceled)

15. (canceled)

16. A method for modulating the endocannabinoid system in a patient in need thereof or in a biological sample, comprising:
administering to said patient or contacting said biological sample with a compound of claim 1, or a physiologically acceptable salt thereof.

17. A method for modulating the endocannabinoid system in a patient in need thereof or in a biological sample, comprising:
administering to said patient or contacting said biological sample with a compound of claim 7, or a physiologically acceptable salt thereof.

18. A method for treating a disorder in a patient in need thereof, comprising:
administering to said patient a compound of claim 1; wherein the disorder is at least one selected from pain, inflammation, anxiety, psychosis, traumatic brain injury, post-traumatic stress disorder, epilepsy, and neurodegenerative disorder.

19. A method for treating a disorder in a patient in need thereof, comprising:
administering to said patient a compound of claim 7; wherein the disorder is at least one selected from pain, inflammation, anxiety, psychosis, traumatic brain injury, post-traumatic stress disorder, epilepsy, and neurodegenerative disorder.

20. A method for treating a disorder in a patient in need thereof, comprising:
administering to said patient a compound of claim 1; wherein the disorder is at least one selected from cannabinoid use disorder, alcohol use disorder and opioid use disorder.

21. A method for treating overdose in a patient in need thereof, comprising:
administering to said patient a compound of claim 1; wherein overdose is at least one selected from cannabinoid overdose and opioid overdose.

22. A method for treating a disorder in a patient in need thereof, comprising:
administering to said patient a compound of claim 7; wherein the disorder is at least one selected from cannabinoid use disorder, alcohol use disorder and opioid use disorder.

23. A method for treating overdose in a patient in need thereof, comprising:
administering to said patient a compound of claim 7; wherein overdose is at least one selected from cannabinoid overdose and opioid overdose.

24. A pharmaceutical composition or formulation, comprising:
(i) a compound of Formula I or Formula II:
(ii) one or more pharmaceutically acceptable polymers;
(iii) optionally one or more pharmaceutically acceptable surfactants and/or one or more pharmaceutically acceptable surfactant-like materials;
(iv) optionally one or more pharmaceutically acceptable carriers and/or one or more pharmaceutically acceptable excipients; and
(v) optionally one or more solvents, in varying ratios.

25. A compound of claim 1, and their pharmaceutically acceptable salts, solvates, hydrates, polymorphs, enantiomers, diastereomers, geometric isomers, racemates, tautomers, rotamers, atropisomers, isotopic variations, or N-oxides thereof comprising a structure selected from the group consisting of: 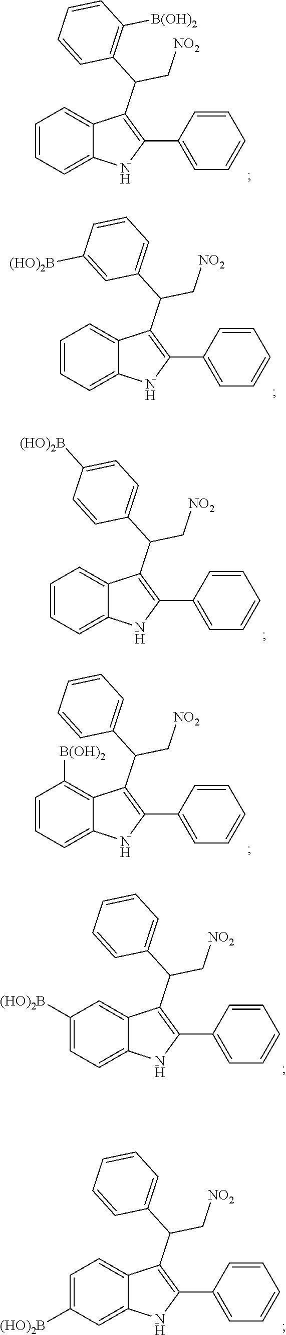 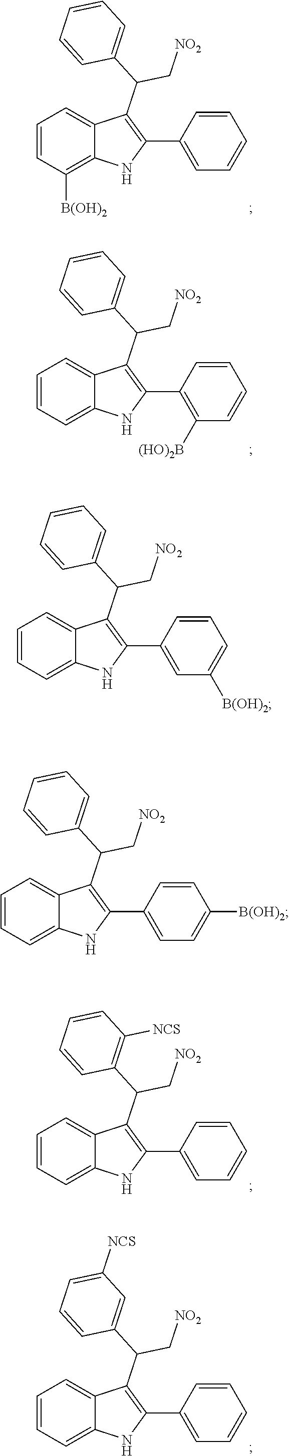 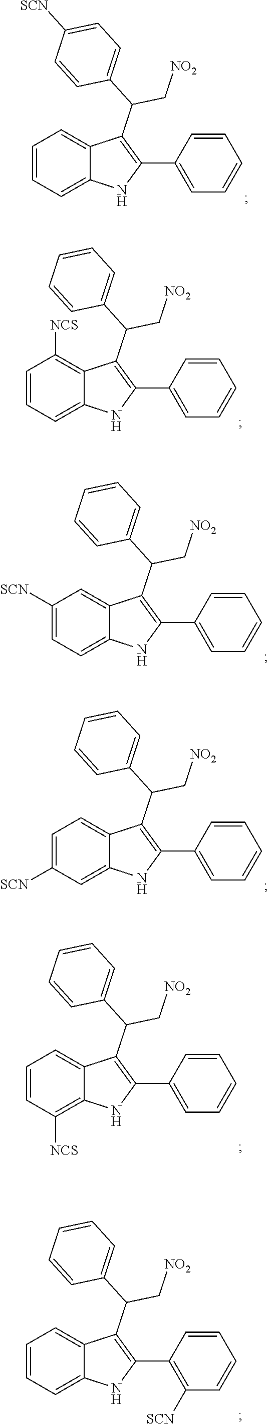 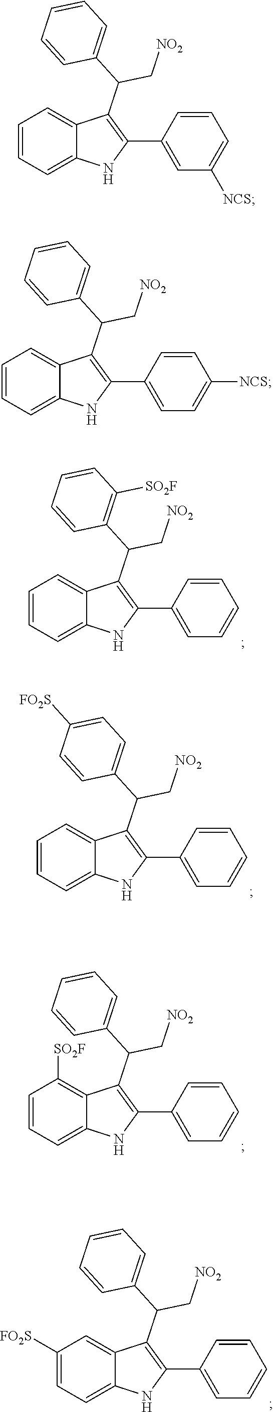 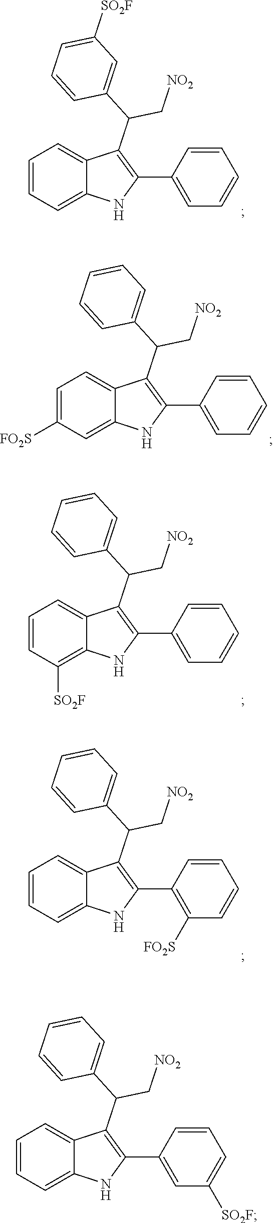 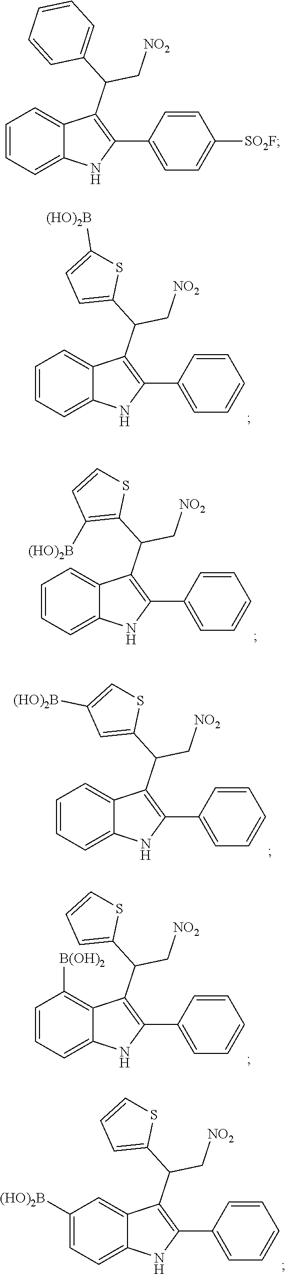 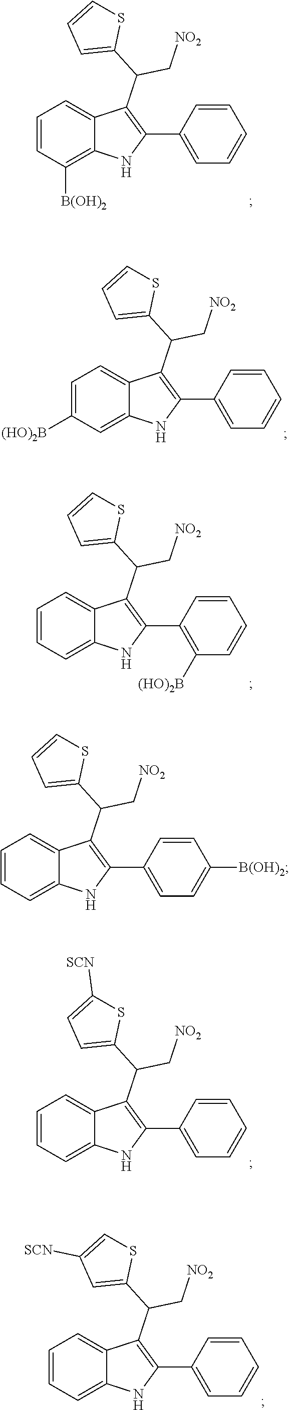 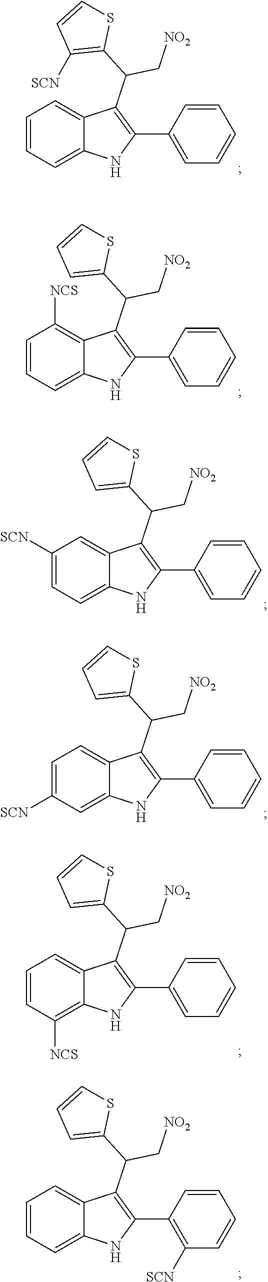 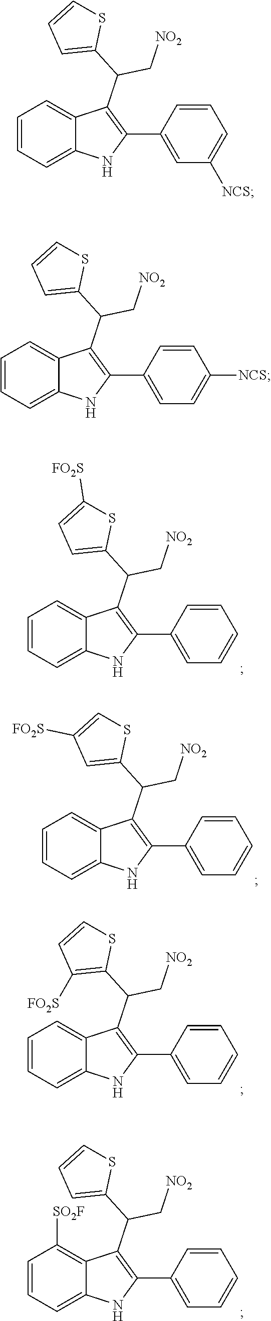 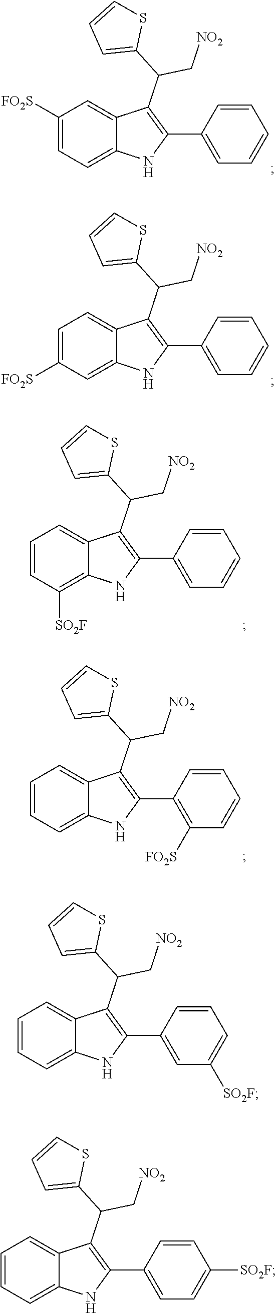 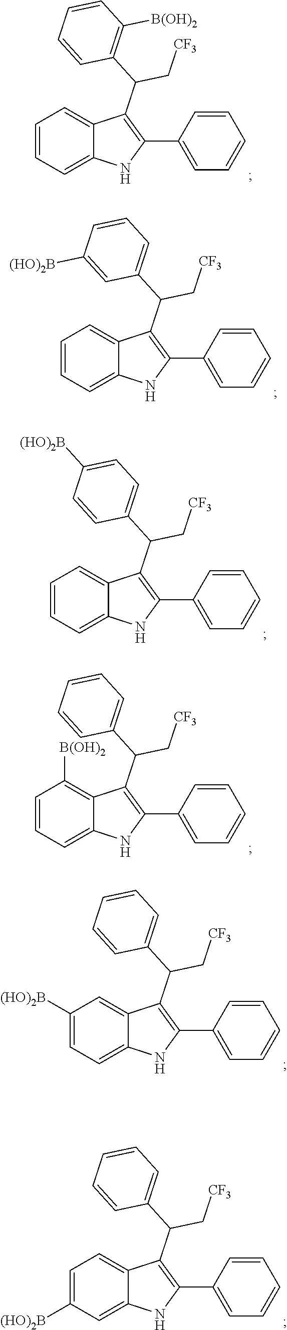 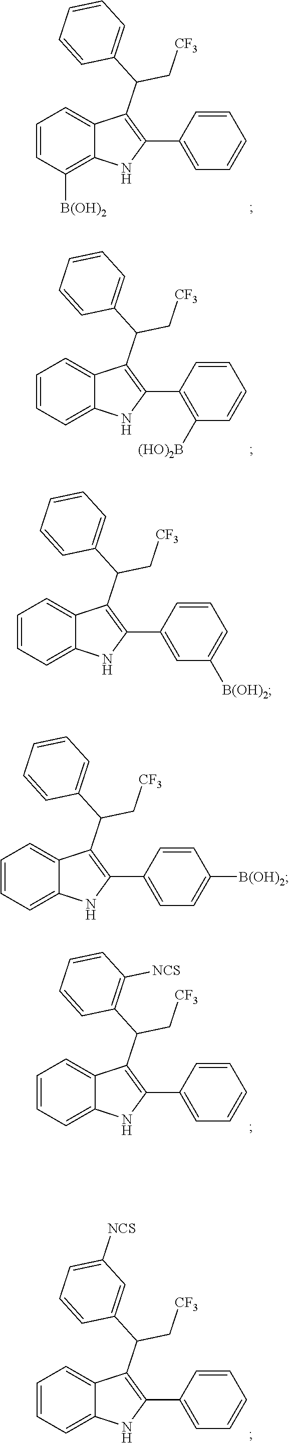 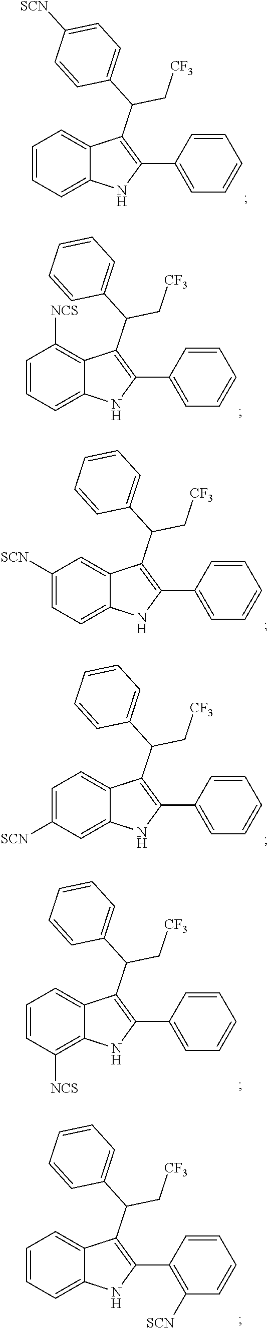 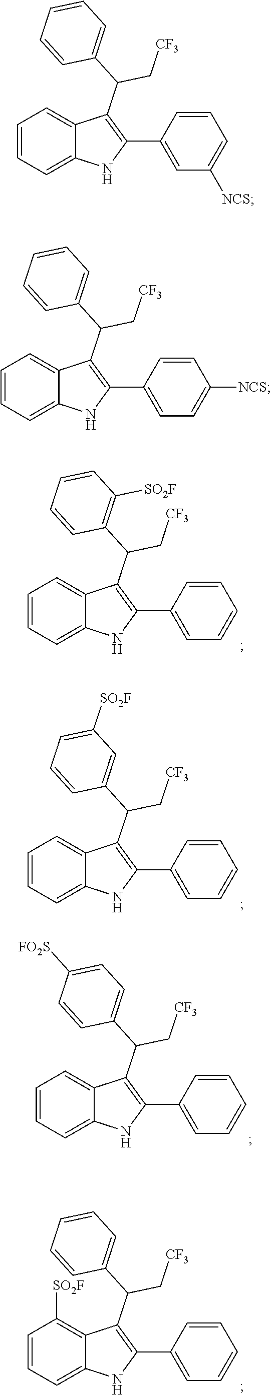 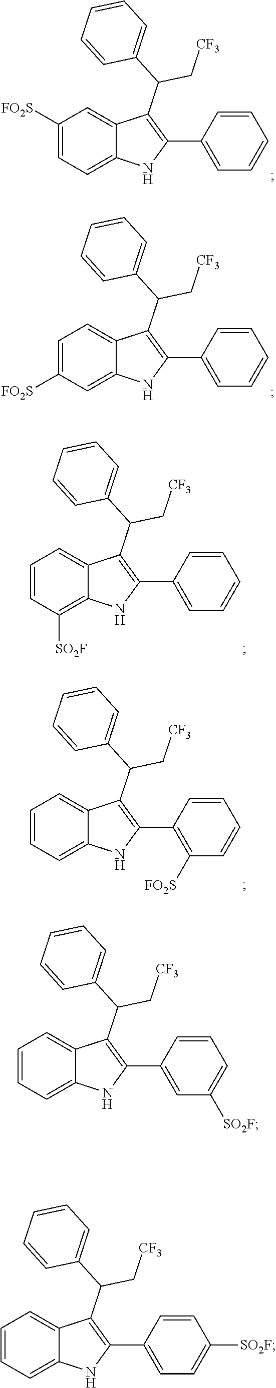 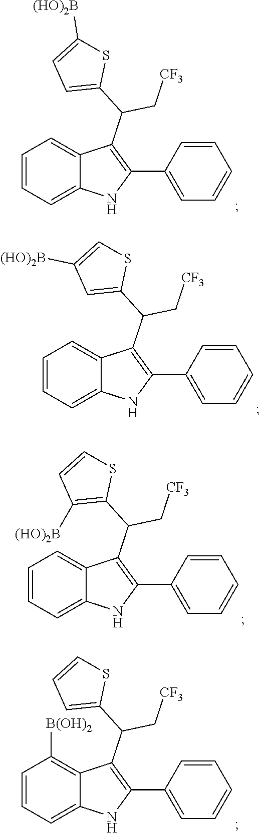 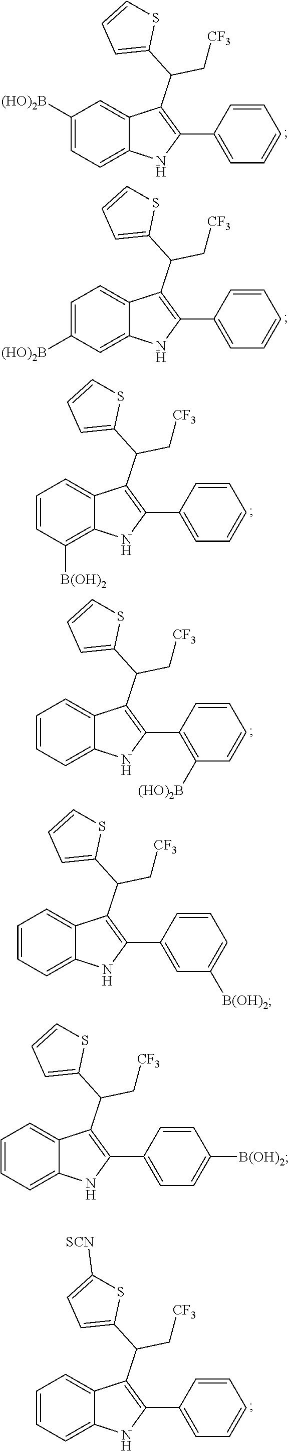 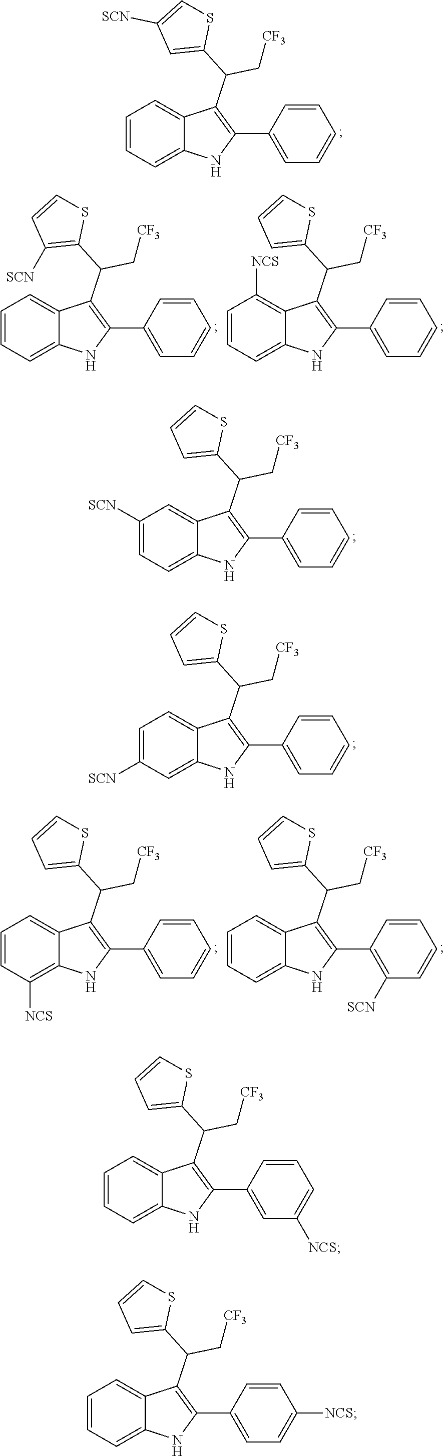 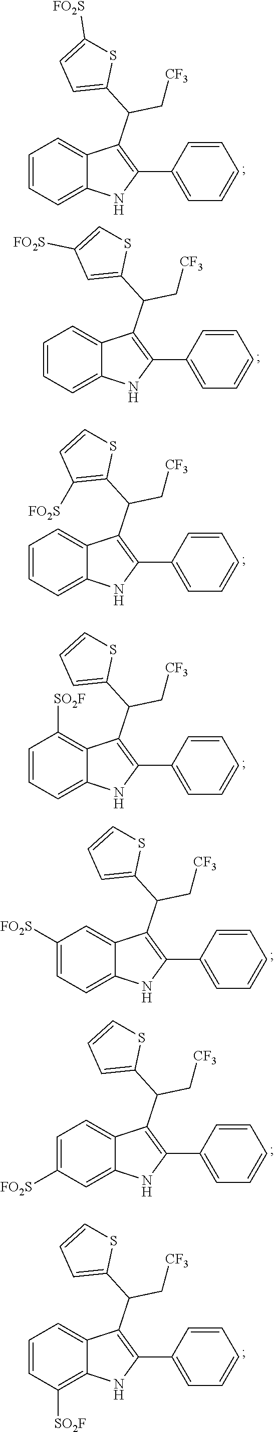 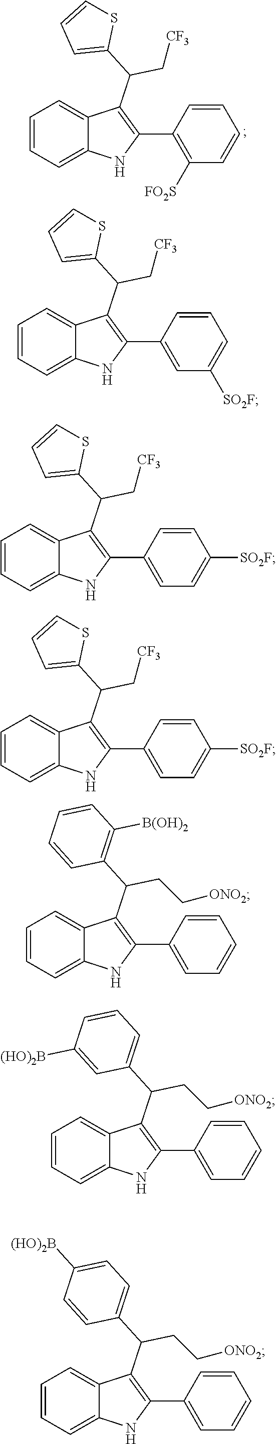 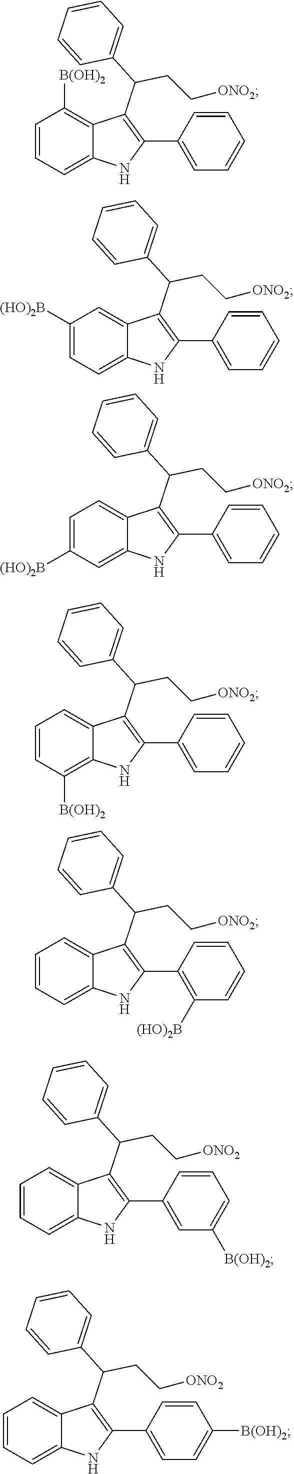 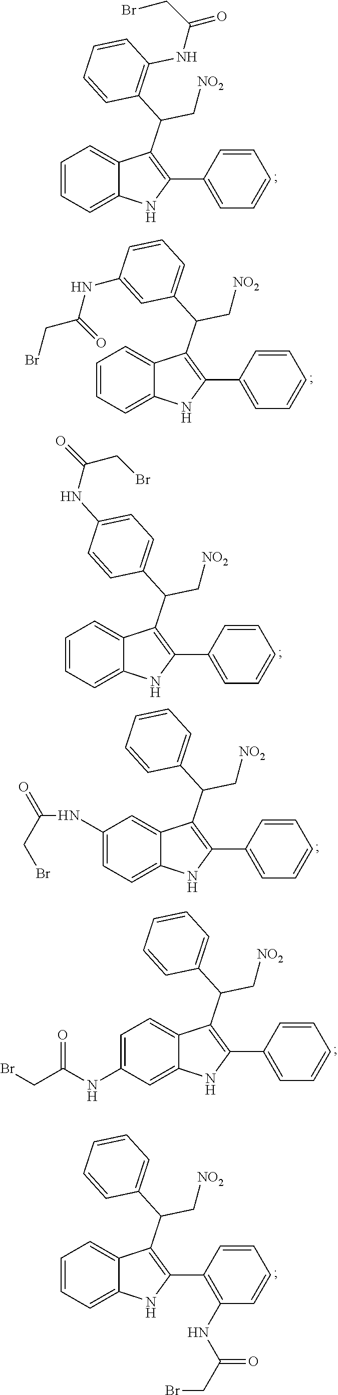 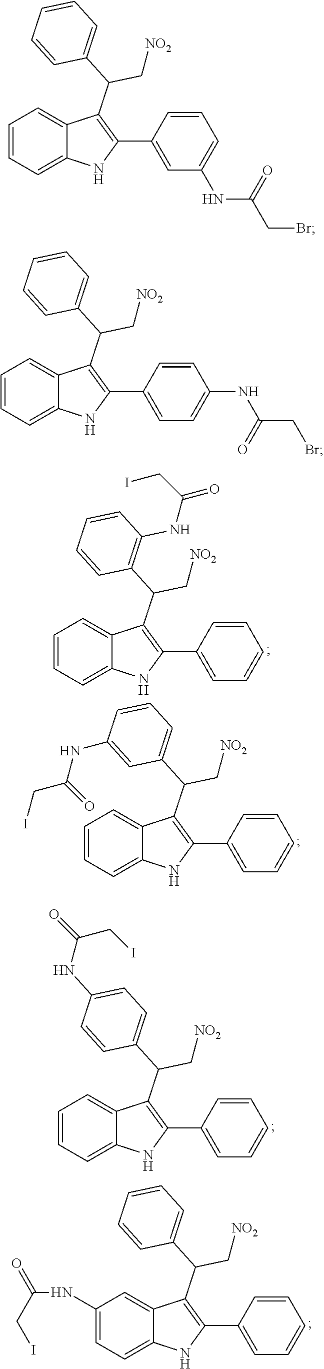 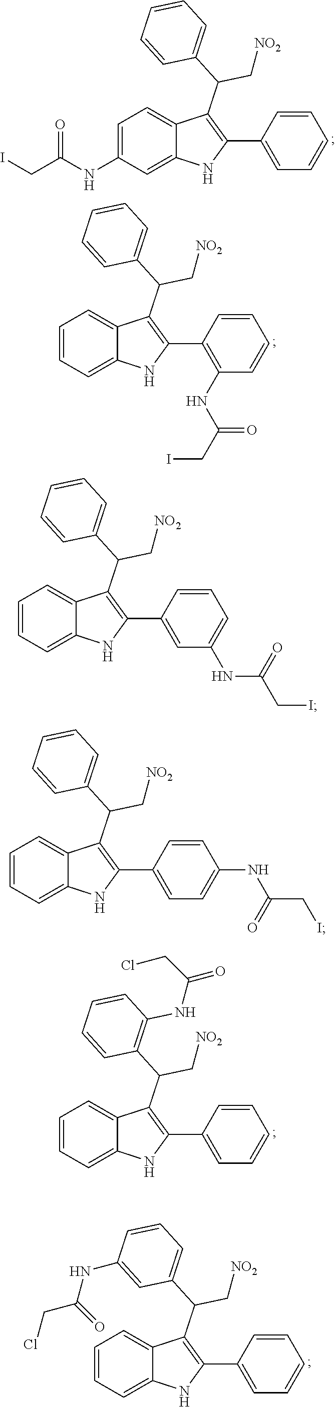 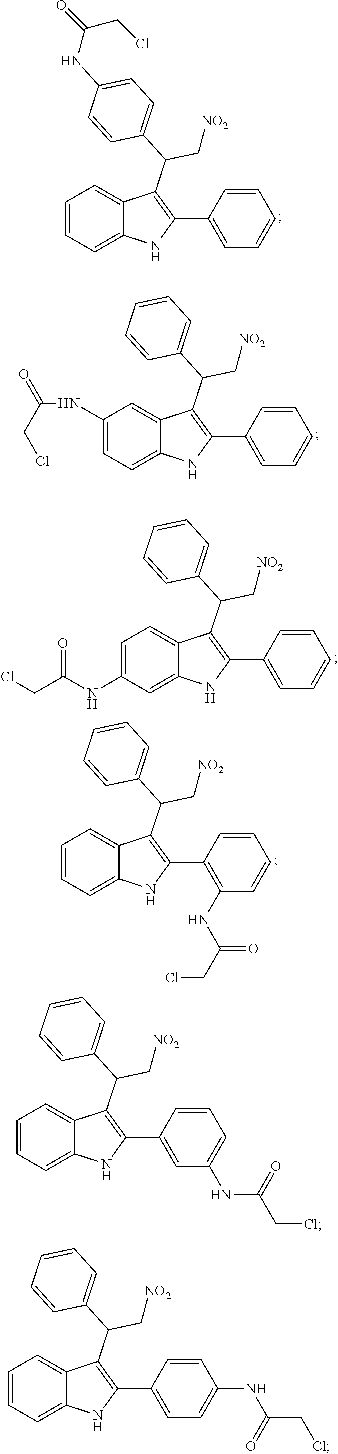 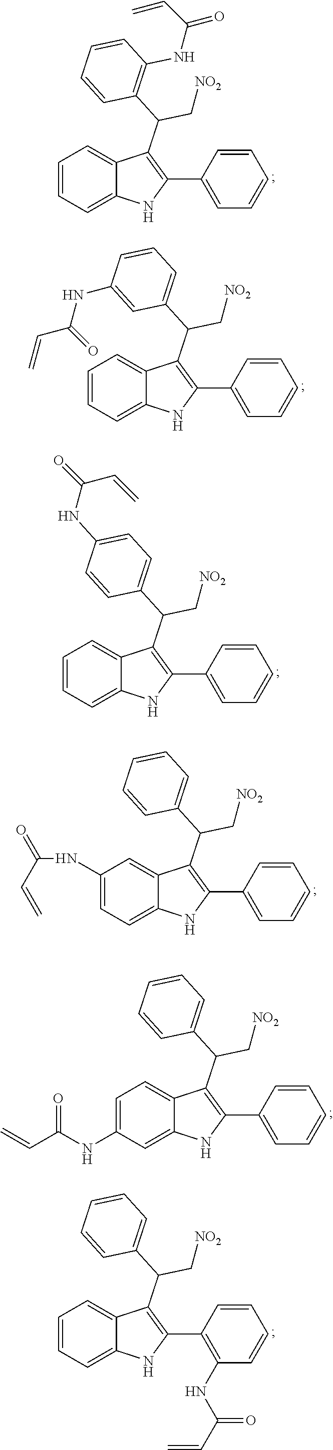 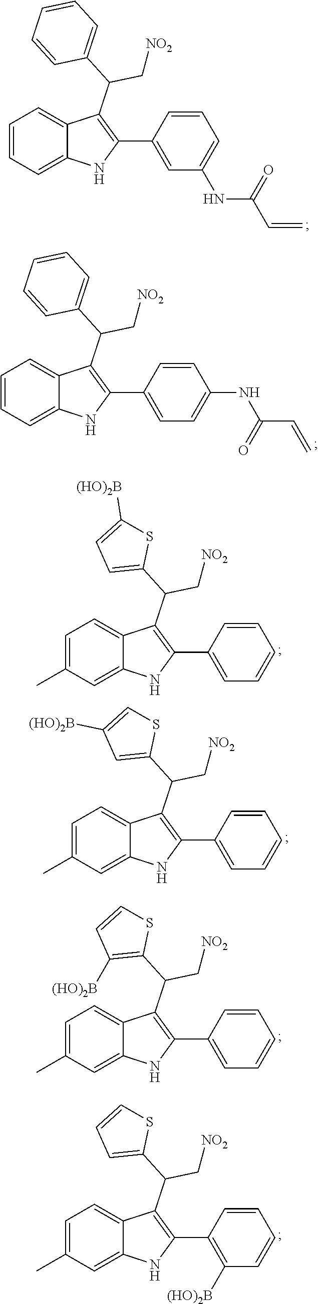 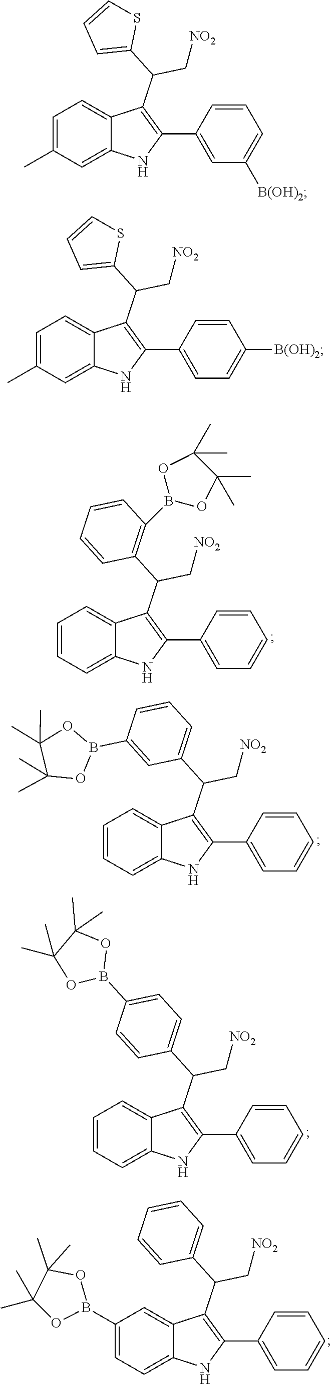 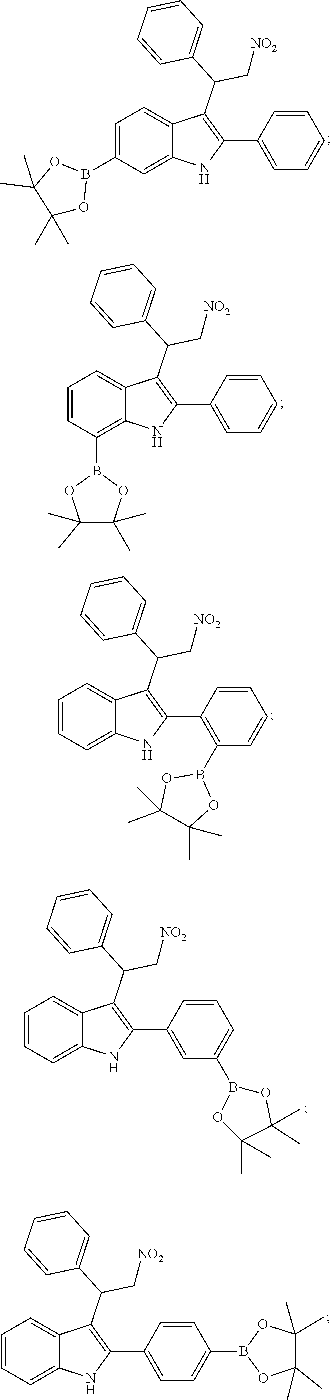 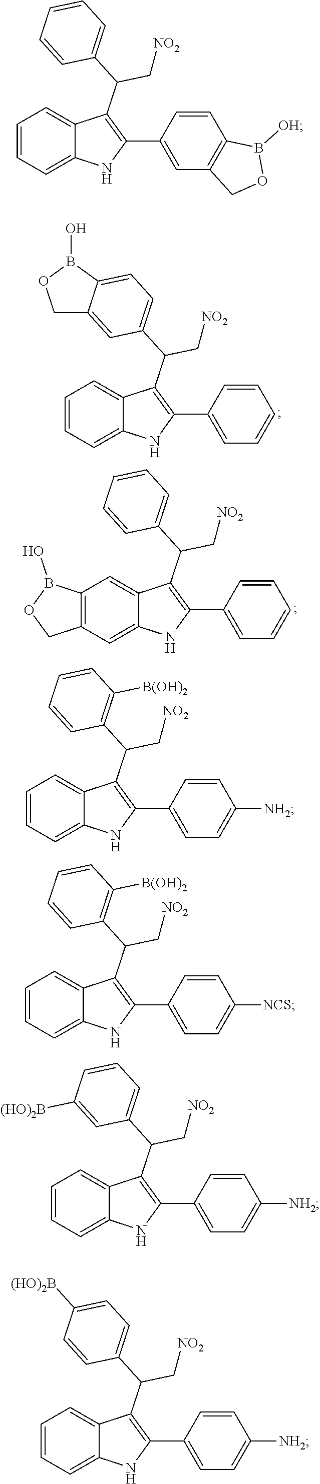 [chemical structure]
